(19) United States
(12) Patent Application Publication
(10) Pub. No.: US 2024/0368263 A1
(43) Pub. Date: Nov. 7, 2024

(54) OPTIMIZED ANTI-TL1A ANTIBODIES

(71) Applicants: PROMETHEUS BIOSCIENCES, INC., San Diego, CA (US); CEDARS-SINAI MEDICAL CENTER, LOS ANGELES, CA (US)

(72) Inventors: Jeffry D. WATKINS, Encinitas, CA (US); Cindy T. DICKERSON, Encinitas, CA (US); J. Monty WATKINS, Encinitas, CA (US); Patricia MCNEELEY, Encinitas, CA (US); Janine Bilsborough, Adelaide (AU); Bradley Henkle, West Hollywood, CA (US); Stephan R. Targan, Santa Monica, CA (US)

(21) Appl. No.: 18/649,944

(22) Filed: Apr. 29, 2024

Related U.S. Application Data
(63) Continuation of application No. 17/050,064, filed on Oct. 23, 2020.
(60) Provisional application No. 62/756,494, filed on Nov. 6, 2018.
(60) Provisional application No. 62/662,605, filed on Apr. 25, 2018.

(51) Int. Cl. C07K 16/24; A61K 9/00; A61K 39/00; A61K 39/395; A61P 1/00; A61P 1/04; C07K 16/28; C12N 5/10; C12N 15/09; C12N 15/63

(52) U.S. Cl. CPC C07K 16/24; A61K 9/0019; A61P 1/00; A61P 1/04; C07K 16/241; C07K 16/28; C07K 16/2875; C12N 5/10; C12N 15/09; C12N 15/63; A61K 39/3955; A61K 2039/505; C07K 2317/24; C07K 2317/56; C07K 2317/565; C07K 2317/71; C07K 2317/732; C07K 2317/734; C07K 2317/76; C07K 2317/90

(57) ABSTRACT

Described herein are humanized anti-TL1A antibodies and pharmaceutical compositions for the treatment of inflammatory bowel disease (IBD), such as Crohn's Disease (CD) and ulcerative colitis (UC).

### CROSS REFERENCE TO RELATED APPLICATIONS

This application is a continuation of U.S. application Ser. No. 17/050,064 filed Oct. 23, 2020, which is a U.S. National Stage of International PCT Application No. PCT/US19/28987 filed Apr. 24, 2019, which claims the benefit of U.S. Provisional Application No. 62/662,605 filed on Apr. 25, 2018, and U.S. Provisional Application No. 62/756,494 filed on Nov. 6, 2018, which applications are incorporated herein by reference.

### INCORPORATION BY REFERENCE OF SEQUENCE LISTING

The present application is being filed along with a Sequence Listing in electronic format. The Sequence Listing is provided as a file entitled 56884-735-304_SL.xml created Apr. 29, 2024, which is 459,786 bytes in size. The information in the electronic format of the Sequence Listing is incorporated by reference in its entirety.

### BACKGROUND

Inflammatory bowel disease (IBD) refers to a collection of intestinal disorders causing inflammatory conditions in the gastrointestinal tract. The primary types of IBD are ulcerative colitis (UC) and Crohn's Disease (CD). These diseases are prevalent, with about 1.86 million people diagnosed globally with UC, and about 1.3 million people diagnosed globally with CD.

Each of these forms has various subclinical phenotypes characteristic of severe forms of IBD that are present in sub-populations of CD and UC patients. One such condition is obstructive Crohn's disease, which can result from long term inflammation that may lead to the formation of scar tissue in the intestinal wall (fibrostenosis) or swelling. Both outcomes can cause narrowing, or obstruction, and are known as either fibrotic or inflammatory strictures. Severe strictures can lead to blockage of the intestine, leading to abdominal pain, bloating, nausea and the inability to pass stool. As another example, penetrating disease phenotypes characterized by bowel obstruction or internal penetrating fistulas, or both, often resulting in complications associated with IBD, including for e.g., intra-abdominal sepsis.

Unfortunately, there are a limited number of therapies available for IBD patients, and the development of new therapeutics has been hampered by sub-optimal results in clinical trials. Existing anti-inflammatory therapy such as steroids and tumor necrosis factor (TNF) inhibitors are typically use as a first line treatment for treating IBD. Unfortunately, a significant number of patients experience a lack of response or a loss of response to existing anti-inflammatory therapies, especially TNF-alpha inhibitors. While the patient is treated with an anti-inflammatory therapy that is ineffective, the disease worsens. Surgery, in the form of structureplasty (reshaping of the intestine) or resection (removal of the intestine), is the only treatment option for patients that do not respond to first line therapies. Surgical treatments for IBD are invasive, causing post-operative risks for an estimated third of patients undergoing surgery, such as anastomotic leak, infection, and bleeding.

The pathogenesis of IBD is thought to involve an uncontrolled immune response that may be triggered by certain environmental factors in a genetically susceptible host. The heterogeneity of disease pathogenesis and clinical course, combined with the variable response to treatment and its associated side effects, suggests a targeted therapeutic approach to treating these diseases is best treatment strategy. Yet there are very few targeted therapies available to IBD patients, especially those patients who may be non-responsive to existing IBD therapies (e.g., anti-TNFa inhibitors). Accordingly, there is a need for novel therapeutics to treat IBD that specifically target enzymes involved in IBD pathogenesis.

### SUMMARY

The present disclosure provides antibodies useful for the treatment of IBD, including moderate to severe forms of IBD characterized by subclinical phenotypes disclosed herein (e.g., refractory disease, stricturing disease, penetrating disease). The antibodies described herein possess superior therapeutic aspects compared to other Tumor necrosis factor ligand 1A (TL1A) binding antibodies. Primarily, the antibodies described herein possess high sequence homology to human germline frameworks while still exhibiting high binding affinity, express at high levels in bacterial and mammalian culture, and possess fewer sequence liabilities, such as deamidation sites, that lead to increased degradation and reduced therapeutic effect.

TL1A and nucleic acids encoding TL1A (Tumor Necrosis Factor Ligand Superfamily Member 15 (TNFSF15) are provided Entrez Gene: 9966; UniProtKB: 095150. TL1A is a proinflammatory molecule which stimulates proliferation and effector functions of CD8 (+) cytotoxic T cells as well as Th1, Th2, and Th17 cells in the presence of TCR stimulation. TL1A is believed to be involved in the pathogenesis of IBD by bridging the innate and adaptive immune response, modulating adaptive immunity by augmenting Th1, Th2, and Th 17 effector cell function, and T-cell accumulation and immunopathology of inflamed tissue.

Certain genotypes containing polymorphisms identified at the TNFSF15 gene, are associated with, and therefore predictive of, a risk of developing IBD (e.g., UC or CD), or a subclinical phenotype of IBD. Expression of TL1A mRNA expression is enriched in patients diagnosed with IBD who carry these risk genotypes. Therefore, inhibiting TL1A expression and/or activity is a promising therapeutic strategy in a variety of T cell-dependent autoimmune diseases, including IBD (e.g., UC and CD).

In one aspect, provided herein, is an antibody or antigen-binding fragment that specifically binds to TL1A, comprising: a heavy chain variable region comprising: (a) an HCDR1 comprising an amino acid sequence set forth by SEQ ID NO: 553; (b) an HCDR2 comprising an amino acid sequence set forth by any one of SEQ ID NOs: 554 to 564 or 574 to 577; and (c) an HCDR3 comprising an amino acid sequence set forth by any one of SEQ ID NOs: 565 to 568 or 578 to 581; and a light chain variable region comprising: (d) an LCDR1 comprising an amino acid sequence set forth by any one of SEQ ID NOs: 569 or 570; (c) an LCDR2 comprising an amino acid sequence set forth by SEQ ID NO: 488; and (f) an LCDR3 comprising an amino acid sequence set forth by any one of SEQ ID NOs: 571 to 573 or 582 to 585. In certain embodiments, the antibody or antigen binding fragment comprises a human heavy chain framework region 1 that is at least 90%, 95%, 96%, 97%, 98%, 99% identical to that set forth is SEQ ID NO: 545. In certain embodiments, the antibody or antigen binding fragment comprises a human heavy chain framework region 2 that is at least 90%, 95%, 96%, 97%, 98%, 99% identical to that set forth is SEQ ID NO: 546. In certain embodiments, the antibody or antigen binding fragment comprises a human heavy chain framework region 3 that is at least 90%, 95%, 96%, 97%, 98%, 99% identical to that set forth is SEQ ID NO: 547 or 586 to 588. In certain embodiments, the antibody or antigen binding fragment comprises a human heavy chain framework region 4 that is at least 90%, 95%, 96%, 97%, 98%, 99% identical to that set forth is SEQ ID NO: 548. In certain embodiments, the antibody or antigen binding fragment comprises a human light chain framework region 1 that is at least 90%, 95%, 96%, 97%, 98%, 99% identical to that set forth is SEQ ID NO: 549. In certain embodiments, the antibody or antigen binding fragment comprises a human light chain framework region 2 that is at least 90%, 95%, 96%, 97%, 98%, 99% identical to that set forth is SEQ ID NO: 550. In certain embodiments, the antibody or antigen binding fragment comprises a human light chain framework region 3 that is at least 90%, 95%, 96%, 97%, 98%, 99% identical to that set forth is SEQ ID NO: 551. In certain embodiments, the antibody or antigen binding fragment comprises a human light chain framework region 4 that is at least 90%, 95%, 96%, 97%, 98%, 99% identical to that set forth is SEQ ID NO: 552. In certain embodiments, the antibody or antigen binding fragment comprises: (a) a human heavy chain framework region 1 that is at least 90%, 95%, 97%, or 98% identical to that set forth is SEQ ID NO: 545; (b) a human heavy chain framework region 2 that is at least 90%, 95%, 97%, or 98% identical to that set forth is SEQ ID NO: 546; (c) a human heavy chain framework region 3 that is at least 90%, 95%, 97%, or 98% identical to that set forth is SEQ ID NO: 547 or 586 to 588; (d) a human heavy chain framework region 4 that is at least 90%, 95%, 97%, or 98% identical to that set forth is SEQ ID NO: 548; (c) a human light chain framework region 1 that is at least 90%, 95%, 97%, or 98% identical to that set forth is SEQ ID NO: 549; (f) a human light chain framework region 2 that is at least 90%, 95%, 97%, or 98% identical to that set forth is SEQ ID NO: 550; (g) a human light chain framework region 3 that is at least 90%, 95%, 97%, or 98% identical to that set forth is SEQ ID NO: 551; and (h) a human light chain framework region 4 that is at least 90%, 95%, 97%, or 98% identical to that set forth is SEQ ID NO: 552. In certain embodiments, the antibody binds human TL1A with a stronger affinity or a 2× stronger affinity compared to the L8 clone as determined by ELISA, wherein the L8 clone comprises a heavy chain variable region amino acid sequence as set forth by SEQ ID NO: 491, and a light chain variable region amino acid sequence as set forth by SEQ ID NO: 490. In certain embodiments, the antibody or antigen-binding fragment is chimeric or humanized. In certain embodiments, the antibody or antigen-binding fragment is an IgG antibody. In certain embodiments, the antibody or antigen-binding fragment comprises a Fab, F (ab) 2,a single-domain antibody, a single chain variable fragment (scFv), or a nanobody. In certain embodiments, the antibody or antigen-binding fragment comprises a heavy chain constant region comprising an amino acid sequence as set forth by SEQ ID NO: 542 or 543. In certain embodiments, the antibody or antigen-binding fragment comprises a heavy chain constant region comprising an amino acid sequence as set forth by SEQ ID NO: 542. In certain embodiments, the antibody or antigen-binding fragment comprises a light chain constant region comprising an amino acid sequence as set forth by SEQ ID NO: 544. In certain embodiments, the antibody or antigen-binding fragment thereof inhibits TL1A induced secretion of interferon gamma from T lymphocytes. In certain embodiments, the antibody or antigen-binding fragment is a component of a pharmaceutical composition comprising the antibody or antigen-binding fragment and a pharmaceutically acceptable excipient, carrier, or diluent. In certain embodiments, the pharmaceutical composition is formulated for intravenous administration. In certain embodiments, antibody or antigen-binding fragment or the pharmaceutical composition is for use in treating inflammatory bowel disease, Crohn's disease, or colitis. In certain embodiments, the antibody or antigen-binding fragment is encoded by a nucleic acid. In certain embodiments, a cell comprises the nucleic acid. In certain embodiments, the cell is a eukaryotic cell. In certain embodiments, the cell is a Chinese Hamster Ovary (CHO) cell. In certain embodiments, described herein, is a method of treating an individual with inflammatory bowel disease, Crohn's disease, or colitis comprising administering an effective amount of the antibody or antigen-binding fragment or the pharmaceutical composition to the individual, wherein the individual is diagnosed with or suspected of being afflicted with inflammatory bowel disease, Crohn's disease, or colitis. In certain embodiments, the antibody or antigen-binding fragment or the pharmaceutical composition is for use in preventing or reducing interferon gamma secretion by T lymphocytes. In certain embodiments, described herein is a method of preventing or reducing interferon gamma secretion by T lymphocytes in an individual comprising administering an effective amount of the antibody or antigen-binding fragment or the pharmaceutical composition to the individual. In certain embodiments, described herein, is a method of preparing an inflammatory bowel disease, Crohn's disease, or colitis treatment comprising incubating the cell comprising the nucleic acid encoding the antibody or antigen-binding fragment into a culture medium under conditions sufficient to secrete the antibody or antigen-binding fragment into the culture medium. In certain embodiments, the method further comprises subjecting the culture medium to at least one purification step. In certain embodiments, described herein, is a method of preparing an inflammatory bowel disease, Crohn's disease, or colitis treatment comprising admixing the antibody or antigen-binding fragment and a pharmaceutically acceptable excipient, carrier, or diluent.

In another aspect, provided herein, is an antibody or antigen-binding fragment that specifically binds to TL1A, comprising: a heavy chain variable region comprising: (a) an HCDR1 comprising an amino acid sequence set forth by SEQ ID NO: 553; (b) an HCDR2 comprising an amino acid sequence set forth by SEQ ID NO: 559; and (c) an HCDR3 comprising an amino acid sequence set forth by SEQ ID NO: 567; and (d) a light chain variable region comprising: (d) an LCDR1 comprising an amino acid sequence set forth by SEQ ID NO: 569; (c) an LCDR2 comprising an amino acid sequence set forth by SEQ ID NO: 488; and (f) an LCDR3 comprising an amino acid sequence set forth by any one of SEQ ID NO: 573. In certain embodiments, the antibody or antigen binding fragment comprises a human heavy chain framework region 1 that is at least 90%, 95%, 96%, 97%, 98%, 99% identical to that set forth is SEQ ID NO: 545. In certain embodiments, the antibody or antigen binding fragment comprises a human heavy chain framework region 2 that is at least 90%, 95%, 96%, 97%, 98%, 99% identical to that set forth is SEQ ID NO: 546. In certain embodiments, the antibody or antigen binding fragment comprises a human heavy chain framework region 3 that is at least 90%, 95%, 96%, 97%, 98%, 99% identical to that set forth is SEQ ID NO: 547 or 586 to 588. In certain embodiments, the antibody or antigen binding fragment comprises a human heavy chain framework region 4 that is at least 90%, 95%, 96%, 97%, 98%, 99% identical to that set forth is SEQ ID NO: 548. In certain embodiments, the antibody or antigen binding fragment comprises a human light chain framework region 1 that is at least 90%, 95%, 96%, 97%, 98%, 99% identical to that set forth is SEQ ID NO: 549. In certain embodiments, the antibody or antigen binding fragment comprises a human light chain framework region 2 that is at least 90%, 95%, 96%, 97%, 98%, 99% identical to that set forth is SEQ ID NO: 550. In certain embodiments, the antibody or antigen binding fragment comprises a human light chain framework region 3 that is at least 90%, 95%, 96%, 97%, 98%, 99% identical to that set forth is SEQ ID NO: 551. In certain embodiments, the antibody or antigen binding fragment comprises a human light chain framework region 4 that is at least 90%, 95%, 96%, 97%, 98%, 99% identical to that set forth is SEQ ID NO: 552. In certain embodiments, the antibody or antigen binding fragment comprises: (a) a human heavy chain framework region 1 that is at least 90%, 95%, 97%, or 98% identical to that set forth is SEQ ID NO: 545; (b) a human heavy chain framework region 2 that is at least 90%, 95%, 97%, or 98% identical to that set forth is SEQ ID NO: 546; (c) a human heavy chain framework region 3 that is at least 90%, 95%, 97%, or 98% identical to that set forth is SEQ ID NO: 547 or 586 to 588; (d) a human heavy chain framework region 4 that is at least 90%, 95%, 97%, or 98% identical to that set forth is SEQ ID NO: 548; (c) a human light chain framework region 1 that is at least 90%, 95%, 97%, or 98% identical to that set forth is SEQ ID NO: 549; (f) a human light chain framework region 2 that is at least 90%, 95%, 97%, or 98% identical to that set forth is SEQ ID NO: 550; (g) a human light chain framework region 3 that is at least 90%, 95%, 97%, or 98% identical to that set forth is SEQ ID NO: 551; and (h) a human light chain framework region 4 that is at least 90%, 95%, 97%, or 98% identical to that set forth is SEQ ID NO: 552. In certain embodiments, the antibody or antigen-binding fragment that specifically binds to TL1A, comprises: (a) a heavy chain variable region comprising an amino acid sequence at least about 85%, 90%, 95%, 97%, 98%, 99%, or 100% identical to SEQ ID NO: 503; and (b) a light chain variable region comprising an amino acid sequence at least about 85%, 90%, 95%, 97%, 98%, 99%, or 100% identical to SEQ ID NO: 502. In certain embodiments, the antibody binds human TL1A with a stronger affinity or a 2× stronger affinity compared to the L8 clone as determined by ELISA, wherein the L8 clone comprises a heavy chain variable region amino acid sequence as set forth by SEQ ID NO: 491, and a light chain variable region amino acid sequence as set forth by SEQ ID NO: 490. In certain embodiments, the antibody or antigen-binding fragment is chimeric or humanized. In certain embodiments, the antibody or antigen-binding fragment is an IgG antibody. In certain embodiments, the antibody or antigen-binding fragment comprises a Fab, F (ab) 2,a single-domain antibody, a single chain variable fragment (scFv), or a nanobody. In certain embodiments, the antibody or antigen-binding fragment comprises a heavy chain constant region comprising an amino acid sequence as set forth by SEQ ID NO: 542 or 543. In certain embodiments, the antibody or antigen-binding fragment comprises a heavy chain constant region comprising an amino acid sequence as set forth by SEQ ID NO: 542. In certain embodiments, the antibody or antigen-binding fragment comprises a light chain constant region comprising an amino acid sequence as set forth by SEQ ID NO: 544. In certain embodiments, the antibody or antigen-binding fragment thereof inhibits TL1A induced secretion of interferon gamma from T lymphocytes. In certain embodiments, the antibody or antigen-binding fragment is a component of a pharmaceutical composition comprising the antibody or antigen-binding fragment and a pharmaceutically acceptable excipient, carrier, or diluent. In certain embodiments, the pharmaceutical composition is formulated for intravenous administration. In certain embodiments, antibody or antigen-binding fragment or the pharmaceutical composition is for use in treating inflammatory bowel disease, Crohn's disease, or colitis. In certain embodiments, the antibody or antigen-binding fragment is encoded by a nucleic acid. In certain embodiments, a cell comprises the nucleic acid. In certain embodiments, the cell is a eukaryotic cell. In certain embodiments, the cell is a Chinese Hamster Ovary (CHO) cell. In certain embodiments, described herein, is a method of treating an individual with inflammatory bowel disease, Crohn's disease, or colitis comprising administering an effective amount of the antibody or antigen-binding fragment or the pharmaceutical composition to the individual, wherein the individual is diagnosed with or suspected of being afflicted with inflammatory bowel disease, Crohn's disease, or colitis. In certain embodiments, the antibody or antigen-binding fragment or the pharmaceutical composition is for use in preventing or reducing interferon gamma secretion by T lymphocytes. In certain embodiments, described herein is a method of preventing or reducing interferon gamma secretion by T lymphocytes in an individual comprising administering an effective amount of the antibody or antigen-binding fragment or the pharmaceutical composition to the individual. In certain embodiments, described herein, is a method of preparing an inflammatory bowel disease, Crohn's disease, or colitis treatment comprising incubating the cell comprising the nucleic acid encoding the antibody or antigen-binding fragment into a culture medium under conditions sufficient to secrete the antibody or antigen-binding fragment into the culture medium. In certain embodiments, the method further comprises subjecting the culture medium to at least one purification step. In certain embodiments, described herein, is a method of preparing an inflammatory bowel disease, Crohn's disease, or colitis treatment comprising admixing the antibody or antigen-binding fragment and a pharmaceutically acceptable excipient, carrier, or diluent.

In another aspect, provided herein, is an antibody or antigen-binding fragment that specifically binds to TL1A, comprising: a heavy chain variable region comprising: (a) an HCDR 1 comprising an amino acid sequence set forth by SEQ ID NO: 553; (b) an HCDR2 comprising an amino acid sequence set forth by SEQ ID NO: 563; and (c) an HCDR3 comprising an amino acid sequence set forth by SEQ ID NO: 568; and a light chain variable region comprising: (d) an LCDR1 comprising an amino acid sequence set forth by SEQ ID NO: 569; (c) an LCDR2 comprising an amino acid sequence set forth by SEQ ID NO: 488; and (f) an LCDR3 comprising an amino acid sequence set forth by any one of SEQ ID NO: 572. In certain embodiments, the antibody or antigen binding fragment comprises a human heavy chain framework region 1 that is at least 90%, 95%, 96%, 97%, 98%, 99% identical to that set forth is SEQ ID NO: 545. In certain embodiments, the antibody or antigen binding fragment comprises a human heavy chain framework region 2 that is at least 90%, 95%, 96%, 97%, 98%, 99% identical to that set forth is SEQ ID NO: 546. In certain embodiments, the antibody or antigen binding fragment comprises a human heavy chain framework region 3 that is at least 90%, 95%, 96%, 97%, 98%, 99% identical to that set forth is SEQ ID NO: 547 or 586 to 588. In certain embodiments, the antibody or antigen binding fragment comprises a human heavy chain framework region 4 that is at least 90%, 95%, 96%, 97%, 98%, 99% identical to that set forth is SEQ ID NO: 548. In certain embodiments, the antibody or antigen binding fragment comprises a human light chain framework region 1 that is at least 90%, 95%, 96%, 97%, 98%, 99% identical to that set forth is SEQ ID NO: 549. In certain embodiments, the antibody or antigen binding fragment comprises a human light chain framework region 2 that is at least 90%, 95%, 96%, 97%, 98%, 99% identical to that set forth is SEQ ID NO: 550. In certain embodiments, the antibody or antigen binding fragment comprises a human light chain framework region 3 that is at least 90%, 95%, 96%, 97%, 98%, 99% identical to that set forth is SEQ ID NO: 551. In certain embodiments, the antibody or antigen binding fragment comprises a human light chain framework region 4 that is at least 90%, 95%, 96%, 97%, 98%, 99% identical to that set forth is SEQ ID NO: 552. In certain embodiments, the antibody or antigen binding fragment comprises: (a) a human heavy chain framework region 1 that is at least 90%, 95%, 97%, or 98% identical to that set forth is SEQ ID NO: 545; (b) a human heavy chain framework region 2 that is at least 90%, 95%, 97%, or 98% identical to that set forth is SEQ ID NO: 546; (c) a human heavy chain framework region 3 that is at least 90%, 95%, 97%, or 98% identical to that set forth is SEQ ID NO: 547 or 586 to 588; (d) a human heavy chain framework region 4 that is at least 90%, 95%, 97%, or 98% identical to that set forth is SEQ ID NO: 548; (c) a human light chain framework region 1 that is at least 90%, 95%, 97%, or 98% identical to that set forth is SEQ ID NO: 549; (f) a human light chain framework region 2 that is at least 90%, 95%, 97%, or 98% identical to that set forth is SEQ ID NO: 550; (g) a human light chain framework region 3 that is at least 90%, 95%, 97%, or 98% identical to that set forth is SEQ ID NO: 551; and (h) a human light chain framework region 4 that is at least 90%, 95%, 97%, or 98% identical to that set forth is SEQ ID NO: 552. In certain embodiments, the antibody or antigen-binding fragment that specifically binds to TL1A, comprises: (a) a heavy chain variable region comprising an amino acid sequence at least about 85%, 90%, 95%, 97%, 98%, 99%, or 100% identical to SEQ ID NO: 511; and (b) a light chain variable region comprising an amino acid sequence at least about 85%, 90%, 95%, 97%, 98%, 99%, or 100% identical to SEQ ID NO: 510. In certain embodiments, the antibody binds human TL1A with a stronger affinity or a 2× stronger affinity compared to the L8 clone as determined by ELISA, wherein the L8 clone comprises a heavy chain variable region amino acid sequence as set forth by SEQ ID NO: 491, and a light chain variable region amino acid sequence as set forth by SEQ ID NO: 490. In certain embodiments, the antibody or antigen-binding fragment is chimeric or humanized. In certain embodiments, the antibody or antigen-binding fragment is an IgG antibody. In certain embodiments, the antibody or antigen-binding fragment comprises a Fab, F (ab) 2,a single-domain antibody, a single chain variable fragment (scFv), or a nanobody. In certain embodiments, the antibody or antigen-binding fragment comprises a heavy chain constant region comprising an amino acid sequence as set forth by SEQ ID NO: 542 or 543. In certain embodiments, the antibody or antigen-binding fragment comprises a heavy chain constant region comprising an amino acid sequence as set forth by SEQ ID NO: 542. In certain embodiments, the antibody or antigen-binding fragment comprises a light chain constant region comprising an amino acid sequence as set forth by SEQ ID NO: 544. In certain embodiments, the antibody or antigen-binding fragment thereof inhibits TL1A induced secretion of interferon gamma from T lymphocytes. In certain embodiments, the antibody or antigen-binding fragment is a component of a pharmaceutical composition comprising the antibody or antigen-binding fragment and a pharmaceutically acceptable excipient, carrier, or diluent. In certain embodiments, the pharmaceutical composition is formulated for intravenous administration. In certain embodiments, antibody or antigen-binding fragment or the pharmaceutical composition is for use in treating inflammatory bowel disease, Crohn's disease, or colitis. In certain embodiments, the antibody or antigen-binding fragment is encoded by a nucleic acid. In certain embodiments, a cell comprises the nucleic acid. In certain embodiments, the cell is a eukaryotic cell. In certain embodiments, the cell is a Chinese Hamster Ovary (CHO) cell. In certain embodiments, described herein, is a method of treating an individual with inflammatory bowel disease, Crohn's disease, or colitis comprising administering an effective amount of the antibody or antigen-binding fragment or the pharmaceutical composition to the individual, wherein the individual is diagnosed with or suspected of being afflicted with inflammatory bowel disease, Crohn's disease, or colitis. In certain embodiments, the antibody or antigen-binding fragment or the pharmaceutical composition is for use in preventing or reducing interferon gamma secretion by T lymphocytes. In certain embodiments, described herein is a method of preventing or reducing interferon gamma secretion by T lymphocytes in an individual comprising administering an effective amount of the antibody or antigen-binding fragment or the pharmaceutical composition to the individual. In certain embodiments, described herein, is a method of preparing an inflammatory bowel disease, Crohn's disease, or colitis treatment comprising incubating the cell comprising the nucleic acid encoding the antibody or antigen-binding fragment into a culture medium under conditions sufficient to secrete the antibody or antigen-binding fragment into the culture medium. In certain embodiments, the method further comprises subjecting the culture medium to at least one purification step. In certain embodiments, described herein, is a method of preparing an inflammatory bowel disease, Crohn's disease, or colitis treatment comprising admixing the antibody or antigen-binding fragment and a pharmaceutically acceptable excipient, carrier, or diluent.

In another aspect, provided herein, is an antibody or antigen-binding fragment that specifically binds to TL1A, comprising: a heavy chain variable region comprising: (a) an HCDR1 comprising an amino acid sequence set forth by SEQ ID NO: 553; (b) an HCDR2 comprising an amino acid sequence set forth by SEQ ID NO: 555; and (c) an HCDR3 comprising an amino acid sequence set forth by SEQ ID NO: 566; and a light chain variable region comprising: (d) an LCDR1 comprising an amino acid sequence set forth by SEQ ID NO: 569; (c) an LCDR2 comprising an amino acid sequence set forth by SEQ ID NO: 488; and (f) an LCDR3 comprising an amino acid sequence set forth by any one of SEQ ID NO: 572. In certain embodiments, the antibody or antigen binding fragment comprises a human heavy chain framework region 1 that is at least 90%, 95%, 96%, 97%, 98%, 99% identical to that set forth is SEQ ID NO: 545. In certain embodiments, the antibody or antigen binding fragment comprises a human heavy chain framework region 2 that is at least 90%, 95%, 96%, 97%, 98%, 99% identical to that set forth is SEQ ID NO: 546. In certain embodiments, the antibody or antigen binding fragment comprises a human heavy chain framework region 3 that is at least 90%, 95%, 96%, 97%, 98%, 99% identical to that set forth is SEQ ID NO: 547 or 586 to 588. In certain embodiments, the antibody or antigen binding fragment comprises a human heavy chain framework region 4 that is at least 90%, 95%, 96%, 97%, 98%, 99% identical to that set forth is SEQ ID NO: 548. In certain embodiments, the antibody or antigen binding fragment comprises a human light chain framework region 1 that is at least 90%, 95%, 96%, 97%, 98%, 99% identical to that set forth is SEQ ID NO: 549. In certain embodiments, the antibody or antigen binding fragment comprises a human light chain framework region 2 that is at least 90%, 95%, 96%, 97%, 98%, 99% identical to that set forth is SEQ ID NO: 550. In certain embodiments, the antibody or antigen binding fragment comprises a human light chain framework region 3 that is at least 90%, 95%, 96%, 97%, 98%, 99% identical to that set forth is SEQ ID NO: 551. In certain embodiments, the antibody or antigen binding fragment comprises a human light chain framework region 4 that is at least 90%, 95%, 96%, 97%, 98%, 99% identical to that set forth is SEQ ID NO: 552. In certain embodiments, the antibody or antigen binding fragment comprises: (a) a human heavy chain framework region 1 that is at least 90%, 95%, 97%, or 98% identical to that set forth is SEQ ID NO: 545; (b) a human heavy chain framework region 2 that is at least 90%, 95%, 97%, or 98% identical to that set forth is SEQ ID NO: 546; (c) a human heavy chain framework region 3 that is at least 90%, 95%, 97%, or 98% identical to that set forth is SEQ ID NO: 547 or 586 to 588; (d) a human heavy chain framework region 4 that is at least 90%, 95%, 97%, or 98% identical to that set forth is SEQ ID NO: 548; (c) a human light chain framework region 1 that is at least 90%, 95%, 97%, or 98% identical to that set forth is SEQ ID NO: 549; (f) a human light chain framework region 2 that is at least 90%, 95%, 97%, or 98% identical to that set forth is SEQ ID NO: 550; (g) a human light chain framework region 3 that is at least 90%, 95%, 97%, or 98% identical to that set forth is SEQ ID NO: 551; and (h) a human light chain framework region 4 that is at least 90%, 95%, 97%, or 98% identical to that set forth is SEQ ID NO: 552. In certain embodiments, the antibody or antigen-binding fragment that specifically binds to TL1A, comprises: (a) a heavy chain variable region comprising an amino acid sequence at least about 85%, 90%, 95%, 97%, 98%, 99%, or 100% identical to SEQ ID NO: 493; and (b) a light chain variable region comprising an amino acid sequence at least about 85%, 90%, 95%, 97%, 98%, 99%, or 100% identical to SEQ ID NO: 492. In certain embodiments, the antibody binds human TL1A with a stronger affinity or a 2× stronger affinity compared to the L8 clone as determined by ELISA, wherein the L8 clone comprises a heavy chain variable region amino acid sequence as set forth by SEQ ID NO: 491, and a light chain variable region amino acid sequence as set forth by SEQ ID NO: 490. In certain embodiments, the antibody or antigen-binding fragment is chimeric or humanized. In certain embodiments, the antibody or antigen-binding fragment is an IgG antibody. In certain embodiments, the antibody or antigen-binding fragment comprises a Fab, F (ab) 2,a single-domain antibody, a single chain variable fragment (scFv), or a nanobody. In certain embodiments, the antibody or antigen-binding fragment comprises a heavy chain constant region comprising an amino acid sequence as set forth by SEQ ID NO: 542 or 543. In certain embodiments, the antibody or antigen-binding fragment comprises a heavy chain constant region comprising an amino acid sequence as set forth by SEQ ID NO: 542. In certain embodiments, the antibody or antigen-binding fragment comprises a light chain constant region comprising an amino acid sequence as set forth by SEQ ID NO: 544. In certain embodiments, the antibody or antigen-binding fragment thereof inhibits TL1A induced secretion of interferon gamma from T lymphocytes. In certain embodiments, the antibody or antigen-binding fragment is a component of a pharmaceutical composition comprising the antibody or antigen-binding fragment and a pharmaceutically acceptable excipient, carrier, or diluent. In certain embodiments, the pharmaceutical composition is formulated for intravenous administration. In certain embodiments, antibody or antigen-binding fragment or the pharmaceutical composition is for use in treating inflammatory bowel disease, Crohn's disease, or colitis. In certain embodiments, the antibody or antigen-binding fragment is encoded by a nucleic acid. In certain embodiments, a cell comprises the nucleic acid. In certain embodiments, the cell is a eukaryotic cell. In certain embodiments, the cell is a Chinese Hamster Ovary (CHO) cell. In certain embodiments, described herein, is a method of treating an individual with inflammatory bowel disease, Crohn's disease, or colitis comprising administering an effective amount of the antibody or antigen-binding fragment or the pharmaceutical composition to the individual, wherein the individual is diagnosed with or suspected of being afflicted with inflammatory bowel disease, Crohn's disease, or colitis. In certain embodiments, the antibody or antigen-binding fragment or the pharmaceutical composition is for use in preventing or reducing interferon gamma secretion by T lymphocytes. In certain embodiments, described herein is a method of preventing or reducing interferon gamma secretion by T lymphocytes in an individual comprising administering an effective amount of the antibody or antigen-binding fragment or the pharmaceutical composition to the individual. In certain embodiments, described herein, is a method of preparing an inflammatory bowel disease, Crohn's disease, or colitis treatment comprising incubating the cell comprising the nucleic acid encoding the antibody or antigen-binding fragment into a culture medium under conditions sufficient to secrete the antibody or antigen-binding fragment into the culture medium. In certain embodiments, the method further comprises subjecting the culture medium to at least one purification step. In certain embodiments, described herein, is a method of preparing an inflammatory bowel disease, Crohn's disease, or colitis treatment comprising admixing the antibody or antigen-binding fragment and a pharmaceutically acceptable excipient, carrier, or diluent.

In another aspect, provided herein, is an antibody or antigen-binding fragment that specifically binds to TL1A, comprising: a heavy chain variable region comprising: (a) an HCDR1 comprising an amino acid sequence set forth by SEQ ID NO: 553; (b) an HCDR2 comprising an amino acid sequence set forth by SEQ ID NO: 558; and (c) an HCDR3 comprising an amino acid sequence set forth by SEQ ID NO: 566; and a light chain variable region comprising: (d) an LCDR1 comprising an amino acid sequence set forth by SEQ ID NO: 569; (c) an LCDR2 comprising an amino acid sequence set forth by SEQ ID NO: 488; and (f) an LCDR3 comprising an amino acid sequence set forth by any one of SEQ ID NO: 572. In certain embodiments, the antibody or antigen binding fragment comprises a human heavy chain framework region 1 that is at least 90%, 95%, 96%, 97%, 98%, 99% identical to that set forth is SEQ ID NO: 545. In certain embodiments, the antibody or antigen binding fragment comprises a human heavy chain framework region 2 that is at least 90%, 95%, 96%, 97%, 98%, 99% identical to that set forth is SEQ ID NO: 546. In certain embodiments, the antibody or antigen binding fragment comprises a human heavy chain framework region 3 that is at least 90%, 95%, 96%, 97%, 98%, 99% identical to that set forth is SEQ ID NO: 547 or 586 to 588. In certain embodiments, the antibody or antigen binding fragment comprises a human heavy chain framework region 4 that is at least 90%, 95%, 96%, 97%, 98%, 99% identical to that set forth is SEQ ID NO: 548. In certain embodiments, the antibody or antigen binding fragment comprises a human light chain framework region 1 that is at least 90%, 95%, 96%, 97%, 98%, 99% identical to that set forth is SEQ ID NO: 549. In certain embodiments, the antibody or antigen binding fragment comprises a human light chain framework region 2 that is at least 90%, 95%, 96%, 97%, 98%, 99% identical to that set forth is SEQ ID NO: 550. In certain embodiments, the antibody or antigen binding fragment comprises a human light chain framework region 3 that is at least 90%, 95%, 96%, 97%, 98%, 99% identical to that set forth is SEQ ID NO: 551. In certain embodiments, the antibody or antigen binding fragment comprises a human light chain framework region 4 that is at least 90%, 95%, 96%, 97%, 98%, 99% identical to that set forth is SEQ ID NO: 552. In certain embodiments, the antibody or antigen binding fragment comprises: (a) a human heavy chain framework region 1 that is at least 90%, 95%, 97%, or 98% identical to that set forth is SEQ ID NO: 545; (b) a human heavy chain framework region 2 that is at least 90%, 95%, 97%, or 98% identical to that set forth is SEQ ID NO: 546; (c) a human heavy chain framework region 3 that is at least 90%, 95%, 97%, or 98% identical to that set forth is SEQ ID NO: 547 or 586 to 588; (d) a human heavy chain framework region 4 that is at least 90%, 95%, 97%, or 98% identical to that set forth is SEQ ID NO: 548; (c) a human light chain framework region 1 that is at least 90%, 95%, 97%, or 98% identical to that set forth is SEQ ID NO: 549; (f) a human light chain framework region 2 that is at least 90%, 95%, 97%, or 98% identical to that set forth is SEQ ID NO: 550; (g) a human light chain framework region 3 that is at least 90%, 95%, 97%, or 98% identical to that set forth is SEQ ID NO: 551; and (h) a human light chain framework region 4 that is at least 90%, 95%, 97%, or 98% identical to that set forth is SEQ ID NO: 552. In certain embodiments, the antibody or antigen-binding fragment that specifically binds to TL1A, comprises: (a) a heavy chain variable region comprising an amino acid sequence at least about 85%, 90%, 95%, 97%, 98%, 99%, or 100% identical to SEQ ID NO: 501; and (b) a light chain variable region comprising an amino acid sequence at least about 85%, 90%, 95%, 97%, 98%, 99%, or 100% identical to SEQ ID NO: 500. In certain embodiments, the antibody binds human TL1A with a stronger affinity or a 2× stronger affinity compared to the L8 clone as determined by ELISA, wherein the L8 clone comprises a heavy chain variable region amino acid sequence as set forth by SEQ ID NO: 491, and a light chain variable region amino acid sequence as set forth by SEQ ID NO: 490. In certain embodiments, the antibody or antigen-binding fragment is chimeric or humanized. In certain embodiments, the antibody or antigen-binding fragment is an IgG antibody. In certain embodiments, the antibody or antigen-binding fragment comprises a Fab, F (ab) 2,a single-domain antibody, a single chain variable fragment (scFv), or a nanobody. In certain embodiments, the antibody or antigen-binding fragment comprises a heavy chain constant region comprising an amino acid sequence as set forth by SEQ ID NO: 542 or 543. In certain embodiments, the antibody or antigen-binding fragment comprises a heavy chain constant region comprising an amino acid sequence as set forth by SEQ ID NO: 542. In certain embodiments, the antibody or antigen-binding fragment comprises a light chain constant region comprising an amino acid sequence as set forth by SEQ ID NO: 544. In certain embodiments, the antibody or antigen-binding fragment thereof inhibits TL1A induced secretion of interferon gamma from T lymphocytes. In certain embodiments, the antibody or antigen-binding fragment is a component of a pharmaceutical composition comprising the antibody or antigen-binding fragment and a pharmaceutically acceptable excipient, carrier, or diluent. In certain embodiments, the pharmaceutical composition is formulated for intravenous administration. In certain embodiments, antibody or antigen-binding fragment or the pharmaceutical composition is for use in treating inflammatory bowel disease. In certain embodiments, the antibody or antigen-binding fragment is encoded by a nucleic acid. In certain embodiments, a cell comprises the nucleic acid. In certain embodiments, the cell is a eukaryotic cell. In certain embodiments, the cell is a Chinese Hamster Ovary (CHO) cell. In certain embodiments, described herein, is a method of treating an individual with inflammatory bowel disease, Crohn's disease, or colitis comprising administering an effective amount of the antibody or antigen-binding fragment or the pharmaceutical composition to the individual, wherein the individual is diagnosed with or suspected of being afflicted with inflammatory bowel disease, Crohn's disease, or colitis. In certain embodiments, the antibody or antigen-binding fragment or the pharmaceutical composition is for use in preventing or reducing interferon gamma secretion by T lymphocytes. In certain embodiments, described herein is a method of preventing or reducing interferon gamma secretion by T lymphocytes in an individual comprising administering an effective amount of the antibody or antigen-binding fragment or the pharmaceutical composition to the individual. In certain embodiments, described herein, is a method of preparing an inflammatory bowel disease, Crohn's disease, or colitis treatment comprising incubating the cell comprising the nucleic acid encoding the antibody or antigen-binding fragment into a culture medium under conditions sufficient to secrete the antibody or antigen-binding fragment into the culture medium. In certain embodiments, the method further comprises subjecting the culture medium to at least one purification step. In certain embodiments, described herein, is a method of preparing an inflammatory bowel disease, Crohn's disease, or colitis treatment comprising admixing the antibody or antigen-binding fragment and a pharmaceutically acceptable excipient, carrier, or diluent.

In another aspect, provided herein, is an antibody or antigen-binding fragment that specifically binds to TL1A, comprising: a heavy chain variable region comprising (a) an HCDR1 comprising an amino acid sequence set forth by SEQ ID NO: 553; (b) an HCDR2 comprising an amino acid sequence set forth by SEQ ID NO: 564; and (c) an HCDR3 comprising an amino acid sequence set forth by SEQ ID NO: 568; and a light chain variable region comprising: (d) an LCDR1 comprising an amino acid sequence set forth by SEQ ID NO: 569; (c) an LCDR2 comprising an amino acid sequence set forth by SEQ ID NO: 488; and (f) an LCDR3 comprising an amino acid sequence set forth by any one of SEQ ID NO: 572. In certain embodiments, the antibody or antigen binding fragment comprises a human heavy chain framework region 1 that is at least 90%, 95%, 96%, 97%, 98%, 99% identical to that set forth is SEQ ID NO: 545. In certain embodiments, the antibody or antigen binding fragment comprises a human heavy chain framework region 2 that is at least 90%, 95%, 96%, 97%, 98%, 99% identical to that set forth is SEQ ID NO: 546. In certain embodiments, the antibody or antigen binding fragment comprises a human heavy chain framework region 3 that is at least 90%, 95%, 96%, 97%, 98%, 99% identical to that set forth is SEQ ID NO: 547 or 586 to 588. In certain embodiments, the antibody or antigen binding fragment comprises a human heavy chain framework region 4 that is at least 90%, 95%, 96%, 97%, 98%, 99% identical to that set forth is SEQ ID NO: 548. In certain embodiments, the antibody or antigen binding fragment comprises a human light chain framework region 1 that is at least 90%, 95%, 96%, 97%, 98%, 99% identical to that set forth is SEQ ID NO: 549. In certain embodiments, the antibody or antigen binding fragment comprises a human light chain framework region 2 that is at least 90%, 95%, 96%, 97%, 98%, 99% identical to that set forth is SEQ ID NO: 550. In certain embodiments, the antibody or antigen binding fragment comprises a human light chain framework region 3 that is at least 90%, 95%, 96%, 97%, 98%, 99% identical to that set forth is SEQ ID NO: 551. In certain embodiments, the antibody or antigen binding fragment comprises a human light chain framework region 4 that is at least 90%, 95%, 96%, 97%, 98%, 99% identical to that set forth is SEQ ID NO: 552. In certain embodiments, the antibody or antigen binding fragment comprises: (a) a human heavy chain framework region 1 that is at least 90%, 95%, 97%, or 98% identical to that set forth is SEQ ID NO: 545; (b) a human heavy chain framework region 2 that is at least 90%, 95%, 97%, or 98% identical to that set forth is SEQ ID NO: 546; (c) a human heavy chain framework region 3 that is at least 90%, 95%, 97%, or 98% identical to that set forth is SEQ ID NO: 547 or 586 to 588; (d) a human heavy chain framework region 4 that is at least 90%, 95%, 97%, or 98% identical to that set forth is SEQ ID NO: 548; (c) a human light chain framework region 1 that is at least 90%, 95%, 97%, or 98% identical to that set forth is SEQ ID NO: 549; (f) a human light chain framework region 2 that is at least 90%, 95%, 97%, or 98% identical to that set forth is SEQ ID NO: 550; (g) a human light chain framework region 3 that is at least 90%, 95%, 97%, or 98% identical to that set forth is SEQ ID NO: 551; and (h) a human light chain framework region 4 that is at least 90%, 95%, 97%, or 98% identical to that set forth is SEQ ID NO: 552. In certain embodiments, the antibody or antigen-binding fragment that specifically binds to TL1A, comprises: (a) a heavy chain variable region comprising an amino acid sequence at least about 85%, 90%, 95%, 97%, 98%, 99%, or 100% identical to SEQ ID NO: 515; and (b) a light chain variable region comprising an amino acid sequence at least about 85%, 90%, 95%, 97%, 98%, 99%, or 100% identical to SEQ ID NO: 514. In certain embodiments, the antibody binds human TL1A with a stronger affinity or a 2× stronger affinity compared to the L8 clone as determined by ELISA, wherein the L8 clone comprises a heavy chain variable region amino acid sequence as set forth by SEQ ID NO: 491, and a light chain variable region amino acid sequence as set forth by SEQ ID NO: 490. In certain embodiments, the antibody or antigen-binding fragment is chimeric or humanized. In certain embodiments, the antibody or antigen-binding fragment is an IgG antibody. In certain embodiments, the antibody or antigen-binding fragment comprises a Fab, F (ab) 2,a single-domain antibody, a single chain variable fragment (scFv), or a nanobody. In certain embodiments, the antibody or antigen-binding fragment comprises a heavy chain constant region comprising an amino acid sequence as set forth by SEQ ID NO: 542 or 543. In certain embodiments, the antibody or antigen-binding fragment comprises a heavy chain constant region comprising an amino acid sequence as set forth by SEQ ID NO: 542. In certain embodiments, the antibody or antigen-binding fragment comprises a light chain constant region comprising an amino acid sequence as set forth by SEQ ID NO: 544. In certain embodiments, the antibody or antigen-binding fragment thereof inhibits TL1A induced secretion of interferon gamma from T lymphocytes. In certain embodiments, the antibody or antigen-binding fragment is a component of a pharmaceutical composition comprising the antibody or antigen-binding fragment and a pharmaceutically acceptable excipient, carrier, or diluent. In certain embodiments, the pharmaceutical composition is formulated for intravenous administration. In certain embodiments, antibody or antigen-binding fragment or the pharmaceutical composition is for use in treating inflammatory bowel disease, Crohn's disease, or colitis. In certain embodiments, the antibody or antigen-binding fragment is encoded by a nucleic acid. In certain embodiments, a cell comprises the nucleic acid. In certain embodiments, the cell is a eukaryotic cell. In certain embodiments, the cell is a Chinese Hamster Ovary (CHO) cell. In certain embodiments, described herein, is a method of treating an individual with inflammatory bowel disease, Crohn's disease, or colitis comprising administering an effective amount of the antibody or antigen-binding fragment or the pharmaceutical composition to the individual, wherein the individual is diagnosed with or suspected of being afflicted with inflammatory bowel disease, Crohn's disease, or colitis. In certain embodiments, the antibody or antigen-binding fragment or the pharmaceutical composition is for use in preventing or reducing interferon gamma secretion by T lymphocytes. In certain embodiments, described herein is a method of preventing or reducing interferon gamma secretion by T lymphocytes in an individual comprising administering an effective amount of the antibody or antigen-binding fragment or the pharmaceutical composition to the individual. In certain embodiments, described herein, is a method of preparing an inflammatory bowel disease, Crohn's disease, or colitis treatment comprising incubating the cell comprising the nucleic acid encoding the antibody or antigen-binding fragment into a culture medium under conditions sufficient to secrete the antibody or antigen-binding fragment into the culture medium. In certain embodiments, the method further comprises subjecting the culture medium to at least one purification step. In certain embodiments, described herein, is a method of preparing an inflammatory bowel disease, Crohn's disease, or colitis treatment comprising admixing the antibody or antigen-binding fragment and a pharmaceutically acceptable excipient, carrier, or diluent.

In another aspect, provided herein, is an antibody or antigen-binding fragment that specifically binds to TL1A, comprising: (a) a heavy chain variable region comprising an HCDR1, an HCDR2, and an HCDR3 from any one of SEQ ID NOs: 491, 493, 495, 497, 499, 501, 503, 505, 507, 509, 511, 513, 515, 517, 519, 521, 523, 525, 527, 529, 531, 533, 535, 537, 539, or 541; and (b) a light chain variable region comprising n LCDR1, an LCDR2, and an LCDR3 from any one of SEQ ID NOs: 490, 492, 494, 496, 498, 500, 502, 504, 506, 508, 510, 512, 514, 516, 518, 520, 522, 524, 526, 528, 530, 532, 534, 536, 538, or 540; wherein the CDRs are defined by the Kabat, Chothia, or IMGT method or a combination thereof. In certain embodiments, the antibody or antigen-binding fragment that specifically binds to TL1A, comprises: (a) a heavy chain variable region comprising an amino acid sequence at least about 85%, 90%, 95%, 97%, 98%, 99%, or 100% identical to any one of SEQ ID NOs: 491, 493, 495, 497, 499, 501, 503, 505, 507, 509, 511, 513, 515, 517, 519, 521, 523, 525, 527, 529, 531, 533, 535, 537, 539, or 541; and (b) a light chain variable region comprising an amino acid sequence at least about 85%, 90%, 95%, 97%, 98%, 99%, or 100% identical to any one of SEQ ID NOs: 490, 492, 494, 496, 498, 500, 502, 504, 506, 508, 510, 512, 514, 516, 518, 520, 522, 524, 526, 528, 530, 532, 534, 536, 538, or 540. In certain embodiments, the antibody binds human TL1A with a stronger affinity or a 2× stronger affinity compared to the L8 clone as determined by ELISA, wherein the L8 clone comprises a heavy chain variable region amino acid sequence as set forth by SEQ ID NO: 491, and a light chain variable region amino acid sequence as set forth by SEQ ID NO: 490. In certain embodiments, the antibody or antigen-binding fragment is chimeric or humanized. In certain embodiments, the antibody or antigen-binding fragment is an IgG antibody. In certain embodiments, the antibody or antigen-binding fragment comprises a Fab, F (ab) 2,a single-domain antibody, a single chain variable fragment (scFv), or a nanobody. In certain embodiments, the antibody or antigen-binding fragment comprises a heavy chain constant region comprising an amino acid sequence as set forth by SEQ ID NO: 542 or 543. In certain embodiments, the antibody or antigen-binding fragment comprises a heavy chain constant region comprising an amino acid sequence as set forth by SEQ ID NO: 542. In certain embodiments, the antibody or antigen-binding fragment comprises a light chain constant region comprising an amino acid sequence as set forth by SEQ ID NO: 544. In certain embodiments, the antibody or antigen-binding fragment is a component of a pharmaceutical composition comprising the antibody or antigen-binding fragment and a pharmaceutically acceptable excipient, carrier, or diluent. In certain embodiments, the pharmaceutical composition is formulated for intravenous administration.

In another aspect, described herein, is a method of treating a disease or a condition in an individual possessing a risk variant associated with the disease or the condition, the method comprising administering an effective amount of an antibody or antigen-binding fragment of this disclosure to the individual possessing a risk variant, wherein the disease or the condition comprises at least one of an inflammatory bowel disease (IBD), Crohn's disease (CD), or colitis. In certain embodiments, the individual possesses a plurality of risk variants. In certain embodiments, the plurality of risk variants is at least 3, 4, 5, or 10 risk variants. In certain embodiments, the risk variant of the plurality of risk variants is associated with a subclinical phenotype of the disease or the condition. In certain embodiments, the disease or the condition is a severe form of the at least one of the IBD, the CD, or the colitis.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts the results of a filter lift assay performed as a qualitative assessment of chimeric 5C3D11 Fab expression and antigen binding. Section A of the filter shows expression of heavy chain 5C3D11, section B of the filter shows expression of light chain 5C3D11, and section C of the filter show binding of 5C3D11 Fab binding to human TL1A antigen.

FIG. 2 depicts binding of chimeric 5C3D11 and humanized clone 12835 antibodies to human TL1A by enzyme-linked immunosorbent assay (ELISA).

FIG. 3 depicts the results of a capture filter lift assay demonstrating high sensitivity and high binding strength of chimeric 5C3D11 for human TL1A.

FIG. 4A depicts the results of an ELISA showing binding of CDR-grafted antibody clones 18-7, 21-3 and humanized clone 12835 to human TL1A.

FIG. 4B depicts the results of an ELISA showing binding of CDR-grafted antibody L8 to human TL1A as compared to binding of humanized clone 12835 to human TL1A.

FIG. 5 depicts the results of an ELISA demonstrating the strong binding of immobilized Fabs (chimeric 5C3D11, humanized clone 12835, clone 18-7, clone 21-3, and CDR graft clone L8) to soluble human TL1A antigen.

FIG. 6A depicts the results of an ELISA demonstrating increased affinity of anti-TL1A antibodies having heavy chain CDR3 mutations H3-7 (V102M)-SEQ ID NOS: 44, 38, H3-7 (V102K)-SEQ ID NOS: 43, 38, and H3-7 (V102Q)-SEQ ID NOS: 45, 38 and humanized clone 12835 to human TL1A as compared to CDR graft (clone L8).

FIG. 6B depicts the results of an ELISA demonstrating increased affinity of an anti-TL1A antibody having a heavy chain CDR3 mutation H3-7 (V102W)-SEQ ID NOS: 46, 38, and humanized clone 12835 to human TL1A as compared to CDR graft clone L8.

FIG. 7A depicts the results of an ELISA demonstrating increased affinity of anti-TL1A antibodies having light chain CDR3 mutations L3-4 (S92D)-SEQ ID NOS: 47, 40, L3-4 (S92E)-SEQ ID NOS: 48, 40, L3-4 (S92H)-SEQ ID NOS: 49, 40, L3-4 (S92N)-SEQ ID NOS: 50, 40, and humanized clone 12835, to human TL1A as compared to CDR graft clone L8.

FIG. 7B depicts the results of an ELISA demonstrating increased affinity of an anti-TL1A antibody having a light chain CDR3 mutation L3-4 (S92Q)-SEQ ID NOS: 51, 40, and humanized clone 12835 to human TL1A as compared to CDR graft clone L8.

FIGS. 8A, 8B and 8C depict ELISAs demonstrating binding of Fabs comprising 5C3D11 CDR variants grafted on human heavy chain germline IGH1-46*02 and human light chain germline IGKV3-20*01 to immobilized human TL1A.

FIGS. 9A and 9B depict ELISAs demonstrating binding of Fabs comprising 5C3D11 CDR variants grafted on human heavy chain germline IGH1-3*01 and human light chain germline IGKV3-20*01 to immobilized human TL1A.

FIGS. 10A and 10B depict ELISAs demonstrating binding of immobilized Fabs comprising 5C3D11 CDR variants grafted on human heavy chain germline IGH1-46*02 and human light chain germline IGKV3-20*01 to soluble, biotinylated human TL1A.

FIGS. 11A and 11B depict ELISAs demonstrating binding of immobilized Fabs comprising 5C3D11 CDR variants grafted on human heavy chain germline IGH1-46*02 and human light chain germline IGKV3-20*01 to soluble, biotinylated human TL1A.

FIGS. 12A and 12B depict ELISAs demonstrating binding of immobilized Fabs comprising 5C3D11 CDR variants grafted on human heavy chain germline IGH1-46*02 and human light chain germline IGKV3-20*01 to soluble, biotinylated human TL1A.

FIGS. 13A and 13B depict ELISAs demonstrating binding of immobilized Fabs comprising 5C3D11 CDR variants grafted on human heavy chain germline IGH1-3*01 and human light chain germline IGKV3-20*01 to soluble, biotinylated human TL1A.

FIGS. 14A and 14B demonstrate binding of Fabs comprising 5C3D11 CDR variants to membrane-associated human TL1A.

FIGS. 15A and 15B depict lack of binding of Fabs comprising 5C3D11 CDR variants grafted on human heavy chain germline IGH1-46*02 and human light chain germline IGKV3-20*01 to TRAIL.

FIGS. 16A and 16B depict lack of binding of Fabs comprising 5C3D11 CDR variants grafted on human heavy chain germline IGH1-46*02 and human light chain germline IGKV3-20*01 to LIGHT.

FIGS. 17A and 17B depict lack of binding of Fabs comprising 5C3D11 CDR variants grafted on human heavy chain germline IGH1-46*02 and human light chain germline IGKV3-20*01 to Fas.

FIG. 18 depicts lack of binding of Fabs comprising 5C3D11 CDR variants grafted on human heavy chain germline IGH1-3*01 and human light chain germline IGKV3-20*01 to TRAIL.

FIG. 19 depicts lack of binding of Fabs comprising 5C3D11 CDR variants grafted on human heavy chain germline IGH1-3*01 and human light chain germline IGKV3-20*01 to LIGHT.

FIG. 20 depicts lack of binding of Fabs comprising 5C3D11 CDR variants grafted on human heavy chain germline IGH1-3*01 and human light chain germline IGKV3-20*01 to Fas.

FIGS. 21A and 21B depict ELISAs demonstrating binding of heavy and light chain variable regions comprising 5C3D11 CDR variants with an IgG1 heavy chain (modified) and kappa light chain constant region (21A), or with an IgG2 heavy chain and kappa light chain constant region (21B) to immobilized human TL1A.

FIGS. 22A and 22B depict ELISAs demonstrating binding of soluble, biotinylated human TL1A to immobilized heavy and light chain variable regions comprising 5C3D11 CDR variants with an IgG1 heavy chain (modified) and kappa light chain constant region (22A), or with an IgG2 heavy chain and kappa light chain constant region (22B).

FIG. 23 demonstrates maintenance of binding of heavy and light chain variable regions comprising 5C3D11 CDR variants with an IgG1 heavy chain (modified) and kappa light chain constant region, or with an IgG2 heavy chain and kappa light chain constant region to a membrane-associated form of human TL1A.

FIGS. 24A, 24B, and 24C depict ELISAs demonstrating lack of binding of heavy and light chain variable regions comprising 5C3D11 CDR variants with an IgG1 heavy chain (modified) and kappa light chain constant region, or with an IgG2 heavy chain and kappa light chain constant region to TNFSF family members Fas (24A), TRAIL (24B), or LIGHT (24C).

FIG. 25 demonstrates the inhibition of cynomolgus TL1A induced IFN-γ production in whole blood by humanized Ig constructs comprising 5C3D11 CDR variants grafted on human heavy chain germline IGH1-46*02 and human light chain germline IGKV3-20*01 with an IgG1 heavy chain (modified) and kappa light chain constant region.

FIGS. 26A, 26B, and 26C illustrate inhibition of TL1A induced IFN-γ production, by antibodies described herein, from human whole blood. Shown are results from 3 different donors (26A), (26B), and (26C), antibody concentration (nanomolar) is shown on the x-axis.

FIGS. 27A, 27B, and 27C illustrate inhibition of TL1A induced IFN-γ production, by antibodies described herein, from cynomolgus monkey whole blood. Shown are results from 3 different donors (27A), (27B), and (27C), antibody concentration (nanomolar) is shown on the x-axis.

### DESCRIPTION OF THE INVENTION

Tumor necrosis factor-like protein 1A (TL1A) has been associated with the development and severity of severe inflammatory bowel disease (IBD), including severe forms of colitis and Crohn's Disease (CD). In addition, preclinical and human genetic association data suggests that TL1A is a potential therapeutic target in Crohn's disease. The present disclosure describes optimized antibodies against TL1A, and offers a novel therapeutic for the treatment of IBD.

Described herein, in one aspect, is an antibody or antigen-binding fragment that specifically binds TL1A, comprising: a heavy chain variable region comprising: (a) an HCDR1 comprising an amino acid sequence set forth by SEQ ID NO: 553; (b) an HCDR2 comprising an amino acid sequence set forth by any one of SEQ ID NOs: 554 to 564 or 574 to 577; and (c) an HCDR3 comprising an amino acid sequence set forth by any one of SEQ ID NOs: 565 to 568 or 578 to 581; and a light chain variable region comprising: (d) an LCDR1 comprising an amino acid sequence set forth by any one of SEQ ID NOs: 569 or 570; (c) an LCDR2 comprising an amino acid sequence set forth by SEQ ID NO: 488; and (f) an LCDR3 comprising an amino acid sequence set forth by any one of SEQ ID NOs: 571 to 573 or 582 to 585.

Described herein, in another aspect, is an antibody or antigen-binding fragment that specifically binds TL1A, comprising: (a) a heavy chain variable region comprising an amino acid sequence at least about 90% identical to any one of SEQ ID NOS: 491, 493, 495, 497, 499, 501, 503, 505, 507, 509, 511, 513, 515, 517, 519, 521, 523, 525, 527, 529, 531, 533, 535, 537, 539, or 541; and (b) a light chain variable region comprising an amino acid sequence at least about 90% identical to any one of SEQ ID NOs: 490, 492, 494, 496, 498, 500, 502, 504, 506, 508, 510, 512, 514, 516, 518, 520, 522, 524, 526, 528, 530, 532, 534, 536, 538, or 540.

In some embodiments, an antibody refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. In some embodiments, an antibody includes intact polyclonal antibodies, intact monoclonal antibodies, antibody fragments (such as Fab, Fab′, F(ab′)₂, and Fv fragments), single chain Fv (scFv) mutants, a CDR-grafted antibody, multispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, radioisotopes, etc.

In some embodiments, one or more amino acid modifications may be introduced into the Fc region of an antibody provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, the antibodies of this disclosure have reduced antibody-dependent cell-mediated cytotoxicity (ADCC) or the reduced ability to fix complement. This is desirable in situations where inhibition of target function is desired, but activation of downstream immune responses may create unwanted side effects. Some Fc regions have a natural lack of effector function (e.g., IgG2, SEQ ID NO: 543), and some Fc regions can comprise mutations that reduce effector functions (e.g., a modified IgG1, SEQ ID NO: 542). In certain embodiments, the antibodies of this disclosure have reduced effector function. In certain embodiments, the antibodies of this disclosure comprise an IgG2 constant region as set forth in SEQ ID NO: 543. In certain embodiments, the antibodies of this disclosure comprise a modified IgG1 constant region as set forth in SEQ ID NO: 542.

In some embodiments, the antibodies of this disclosure are variants that possess some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC), monocytes, macrophages, and Natural Killer (NK) cells.

Antibodies can have increased half-lives and/or improved binding to the neonatal Fc receptor (FcRn) (See e.g., US 2005/0014934). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260 and 5,624,821; and WO94/29351). In certain embodiments, the antibodies of this disclosure have increased serum half-life as a result of alternations to the Fc region. In certain embodiments, the alterations comprise M252Y/S254T/T256E mutations to IgG1, or M428L/N434S mutations to IgG1.

In some embodiments, an antibody comprises an antigen-binding fragment that refers to a portion of an antibody having antigenic determining variable regions of an antibody. Examples of antibody fragments include, but are not limited to Fab, Fab′, F(ab′)₂, and Fv fragments, linear antibodies, single chain antibodies, and multispecific antibodies formed from antibody fragments.

In some embodiments, a humanized antibody refers to forms of non-human (e.g., murine) antibodies having specific immunoglobulin chains, chimeric immunoglobulins, or fragments thereof that contain minimal non-human (e.g., murine) sequences. In a non-limiting example, a humanized antibody comprises less than about 40% non-human sequence in the variable region. In some cases, a humanized antibody comprises less than about 20% non-human sequence in a full length antibody sequence. In some cases, humanized antibodies are human immunoglobulins in which residues from the complementarity determining region (CDR) are replaced by residues from the CDR of a non-human species (e.g., mouse, rat, rabbit, hamster) that have the desired specificity, affinity, and capability.

In some embodiments, chimeric antibodies refer to antibodies wherein the sequence of the immunoglobulin molecule is derived from two or more species. As a non-limiting example, the variable region of both light and heavy chains corresponds to the variable region of antibodies derived from one species of mammals (e.g., mouse, rat, rabbit, etc) with the desired specificity, affinity, and capability while the constant regions are homologous to the sequences in antibodies derived from another (usually human) to avoid eliciting an immune response in that species.

As used herein the term “about” means within 10% of the stated amount.

As used herein “risk variant” means any genetic sequence, typically a DNA sequence, of an individual that increases that individuals risk for developing a phenotype (e.g. inflammatory bowel disease, Crohn's disease, colitis, or subclinical phenotype thereof). Risk variants include without limitation single nucleotide polymorphisms (SNPs), indels of any length, short tandem repeats (STRs), and chromosol translocations, duplications, or deletions. Said risk variants include those variants that are associated with severe forms of inflammatory bowel disease, Crohn's disease, or colitis. Said risk variants include those variants which may indicate that an individual may be refractory to treatment with any current therapy for inflammatory bowel disease, Crohn's disease, or colitis. As contemplated herein risk variants can be used to inform a treatment decision with any of the antibodies described herein.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc MP et al., “IMGT unique numbering for immunoglobulin and T cell receptor variable domains and Ig superfamily V-like domains,” Dev Comp Immunol, 2003 January; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Whitelegg NR and Rees AR, “WAM: an improved algorithm for modelling antibodies on the WEB,” Protein Eng. 2000 December; 13 (12): 819-24 (“AbM” numbering scheme. In certain embodiments, the CDRs of the antibodies described herein can be defined by a method selected from Kabat, Chothia, IMGT, Aho, AbM, or combinations thereof.

In some embodiments, an antibody that specifically binds to a protein indicates that the antibody reacts or associates more frequently, more rapidly, with greater duration, with greater affinity, or with some combination of the above to the protein than with alternative substances, including unrelated proteins.

In some embodiments, the terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as fusion with another polypeptide and/or conjugation, e.g., with a labeling component. Also included within the definition are, for example, polypeptides containing one or more analogs of an amino acid (for example, unnatural amino acids, etc.), as well as other modifications known in the art.

In some embodiments, “polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as, but not limited to methylated nucleotides and their analogs or non-nucleotide components. Modifications to the nucleotide structure may be imparted before or after assembly of the polymer. A polynucleotide may be further modified after polymerization, such as by conjugation with a labeling component.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, the terms “individual” or “subject” are used interchangeably and refer to any animal, including, but not limited to, humans, non-human primates, rodents, and domestic and game animals, which is to be the recipient of a particular treatment. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In various embodiments, a subject can be one who has been previously diagnosed with or identified as suffering from or having a condition in need of treatment. In certain embodiments, the subject is a human. In various other embodiments, the subject previously diagnosed with or identified as suffering from or having a condition may or may not have undergone treatment for a condition. In yet other embodiments, a subject can also be one who has not been previously diagnosed as having a condition (i.e., a subject who exhibits one or more risk factors for a condition). A “subject in need” of treatment for a particular condition can be a subject having that condition, diagnosed as having that condition, or at risk of developing that condition. In some embodiments, the subject is a “patient,” that has been diagnosed with a disease or condition described herein.

In some embodiments, the term “therapeutically effective amount” refers to an amount of an antibody, polypeptide, polynucleotide, small organic molecule, or other drug effective to “treat” a disease or disorder in a subject or mammal. In some cases, therapeutically effective amount of the drug reduces the severity of symptoms of the disease or disorder. In some instances, the disease or disorder comprises inflammatory bowel disease (IBD), Crohn's disease (CD), or ulcerative colitis (UC). In some instances, the IBD, CD, and/or UC are severe or medically refractory forms of the IBD, CD, and/or UC. Non-limiting examples of symptoms of IBD, CD, and/or UC include, but are not limited to, diarrhea, fever, fatigue, abdominal pain, abdominal cramping, inflammation, ulceration, nausea, vomiting, bleeding, blood in stool, reduced appetite, and weight loss.

In some embodiments, the terms, “treat” or “treating” as used herein refer to both therapeutic treatment and prophylactic or preventative measures, wherein the object is to prevent or slow down (lessen) the targeted pathologic condition, prevent the pathologic condition, pursue or obtain good overall survival, or lower the chances of the individual developing the condition even if the treatment is ultimately unsuccessful. In some aspects provided herein, subjects in need of treatment include those already with a disease or condition, as well as those susceptible to develop the disease or condition or those in whom the disease or condition is to be prevented. The disease or condition may comprise an inflammatory disease or condition, fibrostenotic or fibrotic disease, thiopurine toxicity or disease related to thiopurine toxicity, non-response to anti-TNF therapy, steroids or immunomodulators.

### Anti-TL1A Antibodies

Various embodiments provide antibodies that bind to TL1A. In some embodiments, the antibodies specifically bind to soluble TL1A. In some embodiments, the antibodies specifically bind to membrane bound TL1A. In some embodiments, an anti-TL1A antibody is provided having a heavy chain comprising four heavy chain framework regions (HCFR) and three heavy chain complementarity-determining regions (HCDR): HCFR1, HCDR1, HCFR2, HCDR2, HCFR3, HCDR3, and HCFR4; and a light chain comprising four light chain framework regions (LCFR) and three light chain complementarity-determining regions (LCDR): LCFR1, LCDR1, LCFR2, LCDR2, LCFR3, LCDR3, and LCFR4. An anti-TL1A antibody may comprise any region provided herein, for example, as provided in Tables 1, 2, 3, the examples, and SEQ ID NOs: 1 to 54, 490 to 588. In some embodiments, an anti-TL1A antibody comprises a variable domain, e.g., as provided herein, with one or more CDR mutations as shown in Table 2, or 19 to 22. In some embodiments, an anti-TL1A antibody comprises one or more CDRs comprising a sequence shown in Tables 19 to 22.

In certain embodiments, the anti-TL1A antibody comprises CDRs corresponding to those set forth in Tables 19 to 22. In certain embodiments, the anti-TL1A antibody or antigen binding fragment comprises a heavy chain variable region comprising: (a) an HCDR1 comprising the amino acid sequence set forth by SEQ ID NO: 484 (DTYMH); (b) an HCDR2 comprising the amino acid sequence set forth by SEQ ID NO: 485 (PASGH); and (c) an HCDR3 comprising the amino acid sequence set forth by SEQ ID NO: 486 (SGGLPD); and a light chain variable region comprising (d) an LCDR1 comprising the amino acid sequence set forth by SEQ ID NO: 487 (ASSSVSYMY); (c) an LCDR2 comprising the amino acid sequence set forth by SEQ ID NO: 488 (ATSNLAS); and (f) an LCDR3 comprising the amino acid sequence set forth by SEQ ID NO: 489 (GNPRT).

In certain embodiments, described herein, is an antibody or antigen-binding fragment that specifically binds to TL1A, comprising a heavy chain variable region comprising: (a) an HCDR1 comprising an amino acid sequence set forth by SEQ ID NO: 553; (b) an HCDR2 comprising an amino acid sequence set forth by any one of SEQ ID NOS: 554 to 564 or 574 to 577; and (c) an HCDR3 comprising an amino acid sequence set forth by any one of SEQ ID NOs: 565 to 568 or 578 to 581; and a light chain variable region comprising: (d) an LCDR1 comprising an amino acid sequence set forth by any one of SEQ ID NOs: 569 or 570; (e) an LCDR2 comprising an amino acid sequence set forth by SEQ ID NO: 488; and (f) an LCDR3 comprising an amino acid sequence set forth by any one of SEQ ID NOs: 571 to 573 or 582 to 585.

In certain embodiments, described herein, is an antibody or antigen-binding fragment that specifically binds to TL1A, comprising: a heavy chain variable region comprising: (a) an HCDR1 comprising an amino acid sequence set forth by SEQ ID NO: 553; (b) an HCDR2 comprising an amino acid sequence set forth by SEQ ID NO: 559; and (c) an HCDR3 comprising an amino acid sequence set forth by SEQ ID NO: 567; and a light chain variable region comprising: (d) an LCDR1 comprising an amino acid sequence set forth by SEQ ID NO: 569; (c) an LCDR2 comprising an amino acid sequence set forth by SEQ ID NO: 488; and (f) an LCDR3 comprising an amino acid sequence set forth by any one of SEQ ID NO: 573. In certain embodiments, described herein, is an antibody or antigen-binding fragment that specifically binds to TL1A, comprising: a heavy chain variable region comprising an amino acid sequence at least about 85%, 90%, 95%, 97%, 98%, 99%, or 100% identical to SEQ ID NO: 503; and a light chain variable region comprising an amino acid sequence at least about 85%, 90%, 95%, 97%, 98%, 99%, or 100% identical to SEQ ID NO: 502. In certain embodiments, the antibody or antigen binding fragment comprises a kappa light chain constant region and IgG1 heavy chain constant region. In certain embodiments, the antibody or antigen binding fragment comprises a kappa light chain constant region and IgG2 heavy chain constant region.

In certain embodiments, described herein, is an antibody or antigen-binding fragment that specifically binds to TL1A, comprising: a heavy chain variable region comprising: (a) an HCDR1 comprising an amino acid sequence set forth by SEQ ID NO: 553; (b) an HCDR2 comprising an amino acid sequence set forth by SEQ ID NO: 563; and (c) an HCDR3 comprising an amino acid sequence set forth by SEQ ID NO: 568; and a light chain variable region comprising (d) an LCDR1 comprising an amino acid sequence set forth by SEQ ID NO: 569; (e) an LCDR2 comprising an amino acid sequence set forth by SEQ ID NO: 488; and (f) an LCDR3 comprising an amino acid sequence set forth by any one of SEQ ID NO: 572. In certain embodiments, described herein, is an antibody or antigen-binding fragment that specifically binds to TL1A, comprising: a heavy chain variable region comprising an amino acid sequence at least about 85%, 90%, 95%, 97%, 98%, 99%, or 100% identical to SEQ ID NO: 511; and a light chain variable region comprising an amino acid sequence at least about 85%, 90%, 95%, 97%, 98%, 99%, or 100% identical to SEQ ID NO: 510. In certain embodiments, the antibody or antigen binding fragment comprises a kappa light chain constant region and IgG1 heavy chain constant region. In certain embodiments, the antibody or antigen binding fragment comprises a kappa light chain constant region and IgG2 heavy chain constant region.

In certain embodiments, described herein, is an antibody or antigen-binding fragment that specifically binds to TL1A, comprising: a heavy chain variable region comprising: (a) an HCDR1 comprising an amino acid sequence set forth by SEQ ID NO: 553; (b) an HCDR2 comprising an amino acid sequence set forth by SEQ ID NO: 555; and (c) an HCDR3 comprising an amino acid sequence set forth by SEQ ID NO: 566; and a light chain variable region comprising: (d) an LCDR1 comprising an amino acid sequence set forth by SEQ ID NO: 569; (c) an LCDR2 comprising an amino acid sequence set forth by SEQ ID NO: 488; and (f) an LCDR3 comprising an amino acid sequence set forth by any one of SEQ ID NO: 572. In certain embodiments, described herein, is an antibody or antigen-binding fragment that specifically binds to TL1A, comprising: a heavy chain variable region comprising an amino acid sequence at least about 85%, 90%, 95%, 97%, 98%, 99%, or 100% identical to SEQ ID NO: 493; and a light chain variable region comprising an amino acid sequence at least about 85%, 90%, 95%, 97%, 98%, 99%, or 100% identical to SEQ ID NO: 492. In certain embodiments, the antibody or antigen binding fragment comprises a kappa light chain constant region and IgG1 heavy chain constant region. In certain embodiments, the antibody or antigen binding fragment comprises a kappa light chain constant region and IgG2 heavy chain constant region.

In certain embodiments, described herein, is an antibody or antigen-binding fragment that specifically binds to TL1A, comprising: a heavy chain variable region comprising: (a) an HCDR1 comprising an amino acid sequence set forth by SEQ ID NO: 553; (b) an HCDR2 comprising an amino acid sequence set forth by SEQ ID NO: 558; and (c) an HCDR3 comprising an amino acid sequence set forth by SEQ ID NO: 566; and a light chain variable region comprising: (d) an LCDR1 comprising an amino acid sequence set forth by SEQ ID NO: 569; (e) an LCDR2 comprising an amino acid sequence set forth by SEQ ID NO: 488; and (f) an LCDR3 comprising an amino acid sequence set forth by SEQ ID NO: 572. In certain embodiments, described herein, is an antibody or antigen-binding fragment that specifically binds to TL1A, comprising: a heavy chain variable region comprising an amino acid sequence at least about 85%, 90%, 95%, 97%, 98%, 99%, or 100% identical to SEQ ID NO: 501; and a light chain variable region comprising an amino acid sequence at least about 85%, 90%, 95%, 97%, 98%, 99%, or 100% identical to SEQ ID NO: 500. In certain embodiments, the antibody or antigen binding fragment comprises a kappa light chain constant region and IgG1 heavy chain constant region. In certain embodiments, the antibody or antigen binding fragment comprises a kappa light chain constant region and IgG2 heavy chain constant region.

In certain embodiments, described herein, is an antibody or antigen-binding fragment that specifically binds to TL1A, comprising: a heavy chain variable region comprising: (a) an HCDR1 comprising an amino acid sequence set forth by SEQ ID NO: 553; (b) an HCDR2 comprising an amino acid sequence set forth by SEQ ID NO: 564; and (c) an HCDR3 comprising an amino acid sequence set forth by SEQ ID NO: 568; and a light chain variable region comprising: (d) an LCDR1 comprising an amino acid sequence set forth by SEQ ID NO: 569; (c) an LCDR2 comprising an amino acid sequence set forth by SEQ ID NO: 488; and (f) an LCDR3 comprising an amino acid sequence set forth by any one of SEQ ID NO: 572. In certain embodiments, described herein, is an antibody or antigen-binding fragment that specifically binds to TL1A, comprising: a heavy chain variable region comprising an amino acid sequence at least about 85%, 90%, 95%, 97%, 98%, 99%, or 100% identical to SEQ ID NO: 515; and a light chain variable region comprising an amino acid sequence at least about 85%, 90%, 95%, 97%, 98%, 99%, or 100% identical to SEQ ID NO: 514. In certain embodiments, the antibody or antigen binding fragment comprises a kappa light chain constant region and IgG1 heavy chain constant region. In certain embodiments, the antibody or antigen binding fragment comprises a kappa light chain constant region and IgG2 heavy chain constant region.

In certain embodiments, the anti-TL1A antibody or antigen binding fragment comprises a heavy chain variable region comprising n HCDR1, an HCDR2, and an HCDR3 from any one of SEQ ID NOs: 491, 493, 495, 497, 499, 501, 503, 505, 507, 509, 511, 513, 515, 517, 519, 521, 523, 525, 527, 529, 531, 533, 535, 537, 539, or 541; and a light chain variable region comprising n LCDR1, an LCDR2, and an LCDR3 from any one of SEQ ID NOs: 490, 492, 494, 496, 498, 500, 502, 504, 506, 508, 510, 512, 514, 516, 518, 520, 522, 524, 526, 528, 530, 532, 534, 536, 538, or 540, wherein the CDRs are defined by Kabat method, the IMGT method, the Chothia method or a combination thereof. In certain embodiments, the anti-TL1A antibody or antigen binding fragment comprises a heavy chain variable region comprising an amino acid sequence at least about 85%, 90%, 95%, 97%, 98%, 99%, or 100% identical to any one of SEQ ID NOs: 491, 493, 495, 497, 499, 501, 503, 505, 507, 509, 511, 513, 515, 517, 519, 521, 523, 525, 527, 529, 531, 533, 535, 537, 539, or 541; and a light chain variable region comprising an amino acid sequence at least about 85%, 90%, 95%, 97%, 98%, 99%, or 100% identical to any one of SEQ ID NOs: 490, 492, 494, 496, 498, 500, 502, 504, 506, 508, 510, 512, 514, 516, 518, 520, 522, 524, 526, 528, 530, 532, 534, 536, 538, or 540.

In certain embodiments, the anti-TL1A antibody or antigen binding fragment comprises a heavy chain variable region comprising: (a) a human heavy chain framework region 1 that is at least 90%, 95%, 96%, 97%, 98%, or 99% identical to that set forth is SEQ ID NO: 545; (b) an HCDR1 comprising the amino acid sequence set forth by SEQ ID NO: 484 (DTYMH); (c) a human heavy chain framework region 2 that is at least 90%, 95%, 96%, 97%, 98%, or 99% identical to that set forth is SEQ ID NO: 546; (d) an HCDR2 comprising the amino acid sequence set forth by SEQ ID NO: 485 (PASGH); (e) a human heavy chain framework region 3 that is at least 90%, 95%, 96%, 97%, 98%, or 99% identical to that set forth is SEQ ID NO: 547 or 586 to 588; (f) an HCDR3 comprising the amino acid sequence set forth by SEQ ID NO: 486 (SGGLPD); and (g) a human heavy chain framework region 4 that is at least 90%, 95%, 96%, 97%, 98%, or 99% identical to that set forth is SEQ ID NO: 548; and a light chain variable region comprising (h) a human light chain framework region 1 that is at least 90%, 95%, 96%, 97%, 98%, or 99% identical to that set forth is SEQ ID NO: 549; (i) an LCDR1 comprising the amino acid sequence set forth by SEQ ID NO: 487 (ASSSVSYMY); (j) a human light chain framework region 2 that is at least 90%, 95%, 96%, 97%, 98%, or 99% identical to that set forth is SEQ ID NO: 550; (k) an LCDR2 comprising the amino acid sequence set forth by SEQ ID NO: 488 (ATSNLAS); (1) a human light chain framework region 3 that is at least 90%, 95%, 96%, 97%, 98%, or 99% identical to that set forth is SEQ ID NO: 551; (m) an LCDR3 comprising the amino acid sequence set forth by SEQ ID NO: 489 (GNPRT); and (n) a human light chain framework region 4 that is at least 90%, 95%, 96%, 97%, 98%, or 99% identical to that set forth is SEQ ID NO: 552.

TABLE 1
Exemplary anti-TL1A antibodies

| | | | | | Murine |
|---|---|---|---|---|---|
| | | | | | FR |
| | | | | | Back |
| | HC- | HC- | LC- | LC- | Muta- |
| Clone | DNA | protein | DNA | protein | tions |
| Murine | SEQ ID | SEQ ID | SEQ ID | SEQ ID | NA |
| 5C3D11 | NO: 1 | NO: 3 | NO: 4 | NO: 6 | |
| Murine | SEQ ID | SEQ ID | SEQ ID | SEQ ID | NA |
| 5C3D11 | NO: 2 | NO: 3 | NO: 5 | NO: 6 | |
| (codon | | | | | |
| opti- | | | | | |
| mized) | | | | | |
| Chimeric | SEQ ID | SEQ ID | SEQ ID | SEQ ID | NA |
| 5C3D11 | NO: 2 | NO: 3 | NO: 5 | NO:6 | |
| 12835 | SEQ ID | SEQ ID | SEQ ID | SEQ ID | 9 |
| (human- | NO: 25 | NO: 26 | NO: 27 | NO: 28 | |
| ized | | | | | |
| 5C3D11) | | | | | |
| 18-7 | SEQ ID | SEQ ID | SEQ ID | SEQ ID | 2 |
| (CDR- | NO: 35 | NO: 36 | NO: 37 | NO: 38 | |
| grafted | | | | | |
| LC) | | | | | |
| 21-3 | SEQ ID | SEQ ID | SEQ ID | SEQ ID | 2 |
| (CDR- | NO: 39 | NO: 40 | NO: 41 | NO: 42 | |
| grafted | | | | | |
| HC) | | | | | |
| L8 (CDR | SEQ ID | SEQ ID | SEQ ID | SEQ ID | 0 |
| graft) | NO: 39 | NO: 40 | NO: 37 | NO: 38 | |

TABLE 2
Exemplary anti-TL1A CDR sequences

| | SEQ | | |
|---|---|---|---|
| | ID | | |
| CDR | NO | Sequence | Definitions |
| H1 | 150 | GFX₁X₂X₃DX₄X₅X₆H | X₁ = D or E |
| | | | X₂ = I, L, P, or V |
| | | | X₃ = G, Q, S, or V |
| | | | X₄ = A, S, T |
| | | | X₅ = F or Y |
| | | | X₆ = I, L, or M |
| H2 | 12 | RX₁X₂PX₃X₄X₅HX₆ | X₁ = I or L |
| | | KX₇X₈PKFX₉X₁₀ | X₂ = D or E |
| | | | X₃ = A or E |
| | | | X₄ = G or S |
| | | | X₅ = A or G |
| | | | X₆ = I, L, T, or V |
| | | | X₇ = I, L, M, S, T, |
| | | | V, or Y |
| | | | X₈ = D, I, N, R, or |
| | | | S |
| | | | X₉ = Q or R |
| | | | X₁₀ = A, D, E, G, H, |
| | | | K, L, M, N, P, R, S, |
| | | | T, or V |
| H3 | 152 | X₁X₂GX₃PX₄X₅ | X₁ = L or S |
| | | | X₂ = A or G |
| | | | X₃ = A, L, or M |
| | | | X₄ = D or E |
| | | | X₅ = K, M, Q, R, S, |
| | | | T, V, or W |
| L1 | 18 | X₁ASSSVX₂X₃X₄X₅ | X₁ = G, R, or W |
| | | | X₂ = I or S |
| | | | X₃ = F or Y |
| | | | X₄ = L or M |
| | | | X₅ = R or Y |
| L2 | 21 | AX₁X₂X₃LX₄S | X₁ = K or T |
| | | | X₂ = E, P, or S |
| | | | X₃ = L, N, or P |
| | | | X₄ = A or T |
| L3 | 155 | X₁QX₂X₃X₄X₅PRX₆ | X₁ = H, N, Q, or S |
| | | | X₂ = F, H, I, P, R, |
| | | | S, W, or Y |
| | | | X₃ = D, E, H, N, Q, |
| | | | S, or V |
| | | | X₄ = A, D, G, Q, or |
| | | | S |
| | | | X₅ = D, F, H, K, L, |
| | | | M, N, Q, R, S, or T |

In various embodiments, an anti-TL1A antibody binds specifically to the same region of TL1A or binds specifically to a region of TL1A that overlaps with the region of TL1A to which an antibody comprising a heavy chain comprising a sequence at least about 90%, 92%, 95%, 98%, or 100% identical to SEQ ID NO: 3 and a light chain comprising a sequence at least about 90%, 92%, 95%, 98%, or 100% identical to SEQ ID NO: 6, binds specifically. In various embodiments, an anti-TL1A antibody binds specifically to the same region of TL1A or binds specifically to a region of TL1A that overlaps with the region of TL1A to which an antibody comprising a heavy chain comprising a sequence at least about 90%, 92%, 95%, 98%, or 100% identical to SEQ ID NO: 26 and a light chain comprising a sequence at least about 90%, 92%, 95%, 98%, or 100% identical to SEQ ID NO: 28, binds specifically. In various embodiments, an anti-TL1A antibody binds specifically to the same region of TL1A or binds specifically to a region of TL1A that overlaps with the region of TL1A to which an antibody comprising a heavy chain comprising a sequence at least about 90%, 92%, 95%, 98%, or 100% identical to SEQ ID NO: 36 and a light chain comprising a sequence at least about 90%, 92%, 95%, 98%, or 100% identical to SEQ ID NO: 38, binds specifically. In various embodiments, an anti-TL1A antibody binds specifically to the same region of TL1A or binds specifically to a region of TL1A that overlaps with the region of TL1A to which an antibody comprising a heavy chain comprising a sequence at least about 90%, 92%, 95%, 98%, or 100% identical to SEQ ID NO: 40 and a light chain comprising a sequence at least about 90%, 92%, 95%, 98%, or 100% identical to SEQ ID NO: 42, binds specifically. In various embodiments, an anti-TL1A antibody binds specifically to the same region of TL1A or binds specifically to a region of TL1A that overlaps with the region of TL1A to which an antibody comprising a heavy chain comprising a sequence at least about 90%, 92%, 95%, 98%, or 100% identical to SEQ ID NO: 40 and a light chain comprising a sequence at least about 90%, 92%, 95%, 98%, or 100% identical to SEQ ID NO: 38, binds specifically. In various embodiments, an anti-TL1A antibody binds specifically to the same region of TL1A or binds specifically to a region of TL1A that overlaps with the region of TL1A to which an antibody comprising a heavy chain comprising a sequence at least about 90%, 92%, 95%, 98%, or 100% identical to SEQ ID NO: 503 and a light chain comprising a sequence at least about 90%, 92%, 95%, 98%, or 100% identical to SEQ ID NO: 502, binds specifically. In various embodiments, an anti-TL1A antibody binds specifically to the same region of TL1A or binds specifically to a region of TL1A that overlaps with the region of TL1A to which an antibody comprising a heavy chain comprising a sequence at least about 90%, 92%, 95%, 98%, or 100% identical to SEQ ID NO: 511 and a light chain comprising a sequence at least about 90%, 92%, 95%, 98%, or 100% identical to SEQ ID NO: 510, binds specifically. In various embodiments, an anti-TL1A antibody binds specifically to the same region of TL1A or binds specifically to a region of TL1A that overlaps with the region of TL1A to which an antibody comprising a heavy chain comprising a sequence at least about 90%, 92%, 95%, 98%, or 100% identical to SEQ ID NO: 493 and a light chain comprising a sequence at least about 90%, 92%, 95%, 98%, or 100% identical to SEQ ID NO: 492, binds specifically. In various embodiments, an anti-TL1A antibody binds specifically to the same region of TL1A or binds specifically to a region of TL1A that overlaps with the region of TL1A to which an antibody comprising a heavy chain comprising a sequence at least about 90%, 92%, 95%, 98%, or 100% identical to SEQ ID NO: 501 and a light chain comprising a sequence at least about 90%, 92%, 95%, 98%, or 100% identical to SEQ ID NO: 500, binds specifically. In various embodiments, an anti-TL1A antibody binds specifically to the same region of TL1A or binds specifically to a region of TL1A that overlaps with the region of TL1A to which an antibody comprising a heavy chain comprising a sequence at least about 90%, 92%, 95%, 98%, or 100% identical to SEQ ID NO: 515 and a light chain comprising a sequence at least about 90%, 92%, 95%, 98%, or 100% identical to SEQ ID NO: 514, binds specifically.

In various embodiments, the anti-TL1A antibody or fragment has a binding affinity to TL1A of at least about 1E⁻⁷, 1E⁻⁸, 1E⁻⁹, 1E⁻¹⁰, or 1E⁻¹¹. In some cases, the binding affinity is from about 1E⁻⁹ to about 1E⁻¹¹.

Various embodiments provide for an anti-TL1A antibody that binds to the same region of a TL1A protein or portion thereof as a reference antibody, e.g., any anti-TL1A antibody described herein. In some embodiments, the reference antibody comprises the heavy chain CDRs of SEQ ID NOS: 150, 12, and 152 and the light chain CDRs of SEQ ID NOS: 18, 21, and 155.

Non-limiting methods for determining whether an anti-TL1A antibody (i.e. test antibody) binds to the same region of a TL1A protein or portion thereof as an antibody described herein are provided. An exemplary embodiment comprises a competition assay. For instance, the method comprises determining whether the test antibody can compete with binding between the reference antibody and the TL1A protein or portion thereof, or determining whether the reference antibody can compete with binding between the test antibody and the TL1A protein or portion thereof. Exemplary methods include use of surface plasmon resonance to evaluate whether an anti-TL1A antibody can compete with the binding between TL1A and another anti-TL1A antibody. In some cases, surface plasmon resonance is utilized in the competition assay. Non-limiting methods are described in the examples.

The TL1A antibodies described herein bind to specific regions or epitopes of human TL1A. These regions are demonstrated herein as useful to inhibit interferon gamma secretion from T lymphocytes. In certain embodiments, disclosed herein are antibodies that compete for binding TL1A with the antibodies described herein. In certain embodiments, disclosed herein are antibodies that bind the same epitope of TL1A bound by the antibodies described herein. In certain embodiments, disclosed herein are antibodies that bind a discrete epitope that overlaps with an epitope of TL1A bound by an antibody described herein. In certain embodiments, disclosed herein are antibodies that bind the same epitope of TL1A, overlap with the an epitope of TL1A by one or more amino acid residues, or that compete for binding to an epitope of TL1A with an antibody or fragment thereof that comprises a heavy chain variable region comprising the amino acid sequence of SEQ ID NO: 503; and a light chain variable region comprising the amino acid of SEQ ID NO: 502. In certain embodiments, disclosed herein are antibodies that bind the same epitope of TL1A, overlap with the an epitope of TL1A by one or more amino acid residues, or that compete for binding to an epitope of TL1A with an antibody or fragment thereof that comprises a heavy chain variable region comprising the amino acid sequence of SEQ ID NO: 511; and a light chain variable region comprising the amino acid of SEQ ID NO: 510. In certain embodiments, disclosed herein are antibodies that bind the same epitope of TL1A, overlap with the an epitope of TL1A by one or more amino acid residues, or that compete for binding to an epitope of TL1A with an antibody or fragment thereof that comprises a heavy chain variable region comprising the amino acid sequence of SEQ ID NO: 493; and a light chain variable region comprising the amino acid of SEQ ID NO: 492. In certain embodiments, disclosed herein are antibodies that bind the same epitope of TL1A, overlap with the an epitope of TL1A by one or more amino acid residues, or that compete for binding to an epitope of TL1A with an antibody or fragment thereof that comprises a heavy chain variable region comprising the amino acid sequence of SEQ ID NO: 501; and a light chain variable region comprising the amino acid of SEQ ID NO: 500. In certain embodiments, disclosed herein are antibodies that bind the same epitope of TL1A, overlap with the an epitope of TL1A by one or more amino acid residues, or that compete for binding to an epitope of TL1A with an antibody or fragment thereof that comprises the amino acid sequence of SEQ ID NO: 515; and a light chain variable region comprising the amino acid of SEQ ID NO: 514.

### Methods of Generating Antibodies

Various embodiments provide for an antibody that is generated using a polypeptide or a nucleotide sequence. In some embodiments, the antibody is a monoclonal antibody. In some embodiments, the antibody is a human antibody or a humanized antibody. In some embodiments, the antibody is an antibody fragment. For example, the antibody is a Fab, an scFv, or a (Fab)₂. In some embodiments, the antibody is a chimeric antibody.

The antibodies described herein can be assayed for specific binding by any method known in the art. The immunoassays which can be used include, but are not limited to, competitive and non-competitive assay systems using techniques such as BIAcore analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are provided in for e.g., Ausubel et al., eds, 1994, Current Protocols in Molecular Biology, Vol. 1, John Wiley & Sons, Inc., New York.

In various embodiments, the anti-TL1A antibody is an antagonist of a TL1A receptor, such as, but not limited to, DR3 and TR6/DcR3. In certain embodiments, the antibody inhibits at least about 10%, at least about 20%, at least about 30%, at least about 50%, at least about 75%, at least about 90%, or about 100% of one or more activity of the bound TL1A receptor. In certain embodiments, the antibodies inhibit TL1A activation as measured by interferon gamma release in human blood. In certain embodiments, the antibody inhibits interferon gamma release in human blood at an IC₅₀ of between about 1 nanomolar and about 100 picomolar. In certain embodiments, the antibody inhibits interferon gamma release in human blood at an IC₅₀ of between about 500 picomolar and about 100 picomolar. In certain embodiments, the antibody inhibits interferon gamma release in human blood at an IC₅₀ of about 500 picomolar. In certain embodiments, the antibody inhibits interferon gamma release in human blood at an IC₅₀ of about 250 picomolar.

In various embodiments, monoclonal antibodies are prepared using methods known in the art, such as, but not limited to the hybridoma method, where a host animal is immunized, as described above to elicit the production by lymphocytes of antibodies that will specifically bind to an immunizing antigen (Kohler and Milstein (1975) Nature 256:495). Hybridomas produce monoclonal antibodies directed specifically against a chosen antigen. The monoclonal antibodies are purified from the culture medium or ascites fluid by techniques known in the art, when propagated either in vitro or in vivo.

In some embodiments, monoclonal antibodies are made using recombinant DNA methods as described in U.S. Pat. No. 4,816,567. The polynucleotides encoding a monoclonal antibody are isolated from mature B-cells or hybridoma cells. The isolated polynucleotides encoding the heavy and light chains are then cloned into suitable expression vectors, which when transfected into host cells (e.g., E. coli cells, simian COS cells, Chinese hamster ovary (CHO) cells, or myeloma cells) generate monoclonal antibodies. The polynucleotide(s) encoding a monoclonal antibody can further be modified in a number of different manners using recombinant DNA technology to generate alternative antibodies.

In various embodiments, a chimeric antibody, a molecule in which different portions are derived from different animal species, such as those having a variable region derived from a murine monoclonal antibody and a human immunoglobulin constant region (e.g., humanized antibodies) can be generated. Chimeric antibodies can be produced using various techniques such as those set forth in Morrison et al., Proc. Natl. Acad. Sci. 81:851-855 (1984); Neuberger et al., Nature 312:604-608 (1984); Takeda et al., Nature 314:452-454 (1985).

In some embodiments, the anti-TL1A monoclonal antibody is a humanized antibody, to reduce antigenicity and HAMA (human anti-mouse antibody) responses when administered to a human subject. Humanized antibodies can be produced using various techniques known in the art. For example, an antibody is humanized by (1) determining the nucleotide and predicted amino acid sequence of the starting antibody light and heavy variable domains; (2) designing the humanized antibody, e.g., deciding which antibody framework region to use during the humanizing process; (3) the actual humanizing methodologies/techniques; and (4) the transfection and expression of the humanized antibody (see, e.g., U.S. Pat. Nos. 5,585,089; 6,835,823; 6,824,989). In various embodiments, a humanized antibody can be further optimized to decrease potential immunogenicity, while maintaining functional activity, for therapy in humans.

Humanized antibodies can also be made in transgenic mice containing human immunoglobulin loci that are capable, upon immunization, of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016. A humanized antibody may also be obtained by a novel genetic engineering approach that enables production of affinity-matured human-like polyclonal antibodies in large animals such as, for example, rabbits and mice. (Sec, e.g. U.S. Pat. No. 6,632,976).

A fully humanized antibody may be created by first designing a variable region amino acid sequence that contains non-human, e.g., rodent-derived CDRs, embedded in human-derived framework sequences. The non-human CDRs provide the desired specificity. Accordingly, in some cases these residues are included in the design of the reshaped variable region essentially unchanged. In some cases, modifications should therefore be restricted to a minimum and closely watched for changes in the specificity and affinity of the antibody. On the other hand, framework residues in theory can be derived from any human variable region. A human framework sequences should be chosen, which is equally suitable for creating a reshaped variable region and for retaining antibody affinity, in order to create a reshaped antibody which shows an acceptable or an even improved affinity. The human framework may be of germline origin, or may be derived from non-germline (e.g. mutated or affinity matured) sequences. Genetic engineering techniques well known to those in the art, for example, but not limited to, phage display of libraries of human antibodies, transgenic mice, human-human hybridoma, hybrid hybridoma, B cell immortalization and cloning, single-cell RT-PCR or HuRAb Technology, may be used to generate a humanized antibody with a hybrid DNA sequence containing a human framework and a non-human CDR. Methods to obtain “humanized antibodies” are well known to those skilled in the art. (e.g., U.S. Pat. Nos. 5,861,155, 6,479,284, 6,407,213, 5,624,821, 20,031,66871, US20020078757, Queen et al., Proc. Natl. Acad Sci USA, 86:10029-10032 (1989) and Hodgson et al., Bio/Technology, 9:421 (1991)).

In certain embodiments, the anti-TL1A antibody is a human antibody. Human antibodies can be directly prepared using various techniques known in the art. Immortalized human B lymphocytes immunized in vitro or isolated from an immunized individual that produce an antibody directed against a target antigen can be generated (Sec, e.g., Cole et al., Monoclonal Antibodies and Cancer Therapy, Alan R. Liss, p. 77 (1985); Boerner et al., 1991, J. Immunol., 147 (1): 86-95; and U.S. Pat. No. 5,750,373). A human antibody can be selected from a phage library. Techniques for the generation and use of antibody phage libraries are described in U.S. Pat. Nos. 5,969,108, 6,172,197, 5,885,793, 6,521,404; 6,544,731; 6,555,313; 6,582,915; 6,593,081; 6,300,064; 6,653,068; 6,706,484; and 7,264,963; and Rothe ct al., 2007, J. Mol. Bio., doi: 10.1016/j.jmb.2007.12.018.

Chimeric, humanized and human antibodies may be produced by recombinant expression. Recombinant polynucleotide constructs typically include an expression control sequence operably linked to the coding sequences of antibody chains, including naturally-associated or heterologous promoter regions. In certain embodiments, it may be desirable to generate amino acid sequence variants of these humanized antibodies, particularly where these improve the binding affinity or other biological properties of the antibody.

In certain embodiments, an antibody fragment is used to treat and/or ameliorate IBD. Various techniques are known for the production of antibody fragments. Generally, these fragments are derived via proteolytic digestion of intact antibodies (for example Morimoto et al., 1993, Journal of Biochemical and Biophysical Methods 24:107-117; Brennan et al., 1985, Science, 229:81). Fab, Fv, and scFv antibody fragments can all be expressed in and secreted from E. coli or other host cells, thus allowing the production of large amounts of these fragments. Other techniques for the production of antibody fragments will be apparent to the skilled practitioner.

According to the present disclosure, techniques can be adapted for the production of single-chain antibodies specific to TL1A (see e.g., U.S. Pat. No. 4,946,778). In addition, methods can be adapted for the construction of Fab expression libraries (see e.g., Huse, et al., Science 246:1275-1281 (1989)) to allow rapid and effective identification of monoclonal Fab fragments with the desired specificity for TL1A, or derivatives, fragments, analogs or homologs thereof. Antibody fragments may be produced by techniques in the art including, but not limited to: (a) a F(ab′)₂ fragment produced by pepsin digestion of an antibody molecule; (b) a Fab fragment generated by reducing the disulfide bridges of an F(ab′)₂ fragment, (c) a Fab fragment generated by the treatment of the antibody molecule with papain and a reducing agent, and (d) Fv fragments.

Also provided herein are modified antibodies comprising any type of variable region that provides for the association of the antibody with TL1A. Those skilled in the art will appreciate that the modified antibodies may comprise antibodies (e.g., full-length antibodies or immunoreactive fragments thereof) in which at least a fraction of one or more of the constant region domains has been deleted or otherwise altered so as to provide desired biochemical characteristics such as decreasing TL1A. In certain embodiments, the variable regions in both the heavy and light chains are altered by at least partial replacement of one or more CDRs and, if necessary, by partial framework region replacement and sequence changing. In some embodiments, the replaced CDRs may be derived from an antibody of the same class, subclass, from an antibody of a different class, for instance, from an antibody from a different species and/or a combination thereof. In some embodiments, the constant region of the modified antibodies will comprise a human constant region. Modifications to the constant region compatible with this disclosure comprise additions, deletions or substitutions of one or more amino acids in one or more domains.

In various embodiments, the expression of an antibody or antigen-binding fragment thereof as described herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. In other embodiments, the antibody or antigen-fragment thereof as described herein may be transfected into the host.

In some embodiments, the expression vectors are transfected into the recipient cell line for the production of the chimeric, humanized, or composite human antibodies described herein. In various embodiments, mammalian cells can be useful as hosts for the production of antibody proteins, which can include, but are not limited to cells of fibroblast origin, such as Vero (ATCC CRL 81) or CHO-K1 (ATCC CRL 61) cells, HeLa cells and L cells. Exemplary eukaryotic cells that can be used to express polypeptides include, but are not limited to, COS cells, including COS 7 cells; 293 cells, including 293-6E cells; CHO cells, including CHO—S and DG44 cells; PER.C6TM cells (Crucell); and NSO cells. In some embodiments, a particular eukaryotic host cell is selected based on its ability to make desired post-translational modifications to the heavy chains and/or light chains.

A number of suitable host cell lines capable of secreting intact heterologous proteins have been developed in the art, and include, but are not limited to CHO cell lines, various COS cell lines, HeLa cells, L cells and multiple myeloma cell lines.

An expression vector carrying a chimeric, humanized, or composite human antibody construct, antibody or antigen-binding fragment thereof as described herein can be introduced into an appropriate host cell by any of a variety of suitable means, depending on the type of cellular host including, but not limited to transformation, transfection, lipofection, conjugation, electroporation, direct microinjection, and microprojectile bombardment, as known to one of ordinary skill in the art. Expression vectors for these cells can include expression control sequences, such as an origin of replication sites, a promoter, an enhancer and necessary processing information sites, such as ribosome binding sites, RNA splice sites, polyadenylation sites, and transcriptional terminator sequences.

In various embodiments, yeast can also be utilized as hosts for the production of the antibody molecules or peptides described herein. In various other embodiments, bacterial strains can also be utilized as hosts for the production of the antibody molecules or peptides described herein. Examples of bacterial strains include, but are not limited to E. coli, Bacillus species, enterobacteria, and various Pseudomonas species.

In some embodiments, one or more antibodies or antigen-binding fragments thereof as described herein can be produced in vivo in an animal that has been engineered (transgenic) or transfected with one or more nucleic acid molecules encoding the polypeptides, according to any suitable method. For production of transgenic animals, transgenes can be microinjected into fertilized oocytes, or can be incorporated into the genome of embryonic stem cells, and the nuclei of such cells transferred into enucleated oocytes. Once expressed, antibodies can be purified according to standard procedures of the art, including HPLC purification, column chromatography, gel electrophoresis and the like (see generally, Scopes, Protein Purification (Springer-Verlag, NY, 1982)).

Once expressed in the host, the whole antibodies, antibody-fragments (e.g., individual light and heavy chains), or other immunoglobulin forms of the present disclosure can be recovered and purified by known techniques, e.g., immunoabsorption or immunoaffinity chromatography, chromatographic methods such as HPLC (high performance liquid chromatography), ammonium sulfate precipitation, gel electrophoresis, or any combination of these. See generally, Scopes, PROTEIN PURIF. (Springer-Verlag, NY, 1982). Substantially pure immunoglobulins of at least about 90% to 95% homogeneity are advantageous, as are those with 98% to 99% or more homogeneity, particularly for pharmaceutical uses. Once purified, partially or to homogeneity as desired, a humanized or composite human antibody can then be used therapeutically or in developing and performing assay procedures, immunofluorescent stainings, etc. See generally, Vols. I & II Immunol. Meth. (Lefkovits & Pernis, eds., Acad. Press, NY, 1979 and 1981).

Various embodiments provide for a genetic construct comprising a nucleic acid encoding an anti-TL1A antibody or fragment provided herein. Genetic constructs of the antibody can be in the form of expression cassettes, which can be suitable for expression of the encoded anti-TL1A antibody or fragment. The genetic construct may be introduced into a host cell with or without being incorporated in a vector. For example, the genetic construct can be incorporated within a liposome or a virus particle. Alternatively, a purified nucleic acid molecule can be inserted directly into a host cell by methods known in the art. The genetic construct can be introduced directly into cells of a host subject by transfection, infection, electroporation, cell fusion, protoplast fusion, microinjection or ballistic bombardment.

Various embodiments provide a recombinant vector comprising the genetic construct of an antibody provided herein. The recombinant vector can be a plasmid, cosmid or phage. The recombinant vectors can include other functional elements; for example, a suitable promoter to initiate gene expression.

Various embodiments provide a host cell comprising a genetic construct and/or recombinant vector described herein.

Various host systems are also advantageously employed to express recombinant protein. Examples of suitable mammalian host cell lines include the COS-7 lines of monkey kidney cells, and other cell lines capable of expressing an appropriate vector including, for example, L cells, C127, 3T3, Chinese hamster ovary (CHO), HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences.

The proteins produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography. Recombinant protein produced in bacterial culture can be isolated. Methods known in the art for purifying antibodies and other proteins also include, for example, those described in U.S. Patent Publication No. 2008/0177048, and 2009/0187005.

One of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retain the ability to specifically bind the target antigen. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as He, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained.

Particular conservative substitutions include, for example; Ala into Gly or into Ser; Arg into Lys; Asn into Gin or into H is; Asp into Glu; Cys into Ser; Gin into Asn; Glu into Asp; Gly into Ala or into Pro; His into Asn or into Gin; lie into Leu or into Val; Leu into lie or into Val; Lys into Arg, into Gin or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu.

In some embodiments, the antibody and/or antigen-binding fragment thereof described herein can be a variant of a sequence described herein, e.g., a conservative substitution variant of an antibody polypeptide. In some embodiments, the variant is a conservatively modified variant. A variant may refer to a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity, e.g., antigen-specific binding activity for the relevant target polypeptide.

Alterations of the native amino acid sequence can be accomplished by any of a number of techniques known to one of skill in the art. Mutations can be introduced at particular loci or by oligonucleotide-directed site-specific mutagenesis procedures. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462.

Nucleic acid molecules encoding amino acid sequence variants of antibodies are prepared by a variety of methods known in the art. These methods include, but are not limited to, preparation by oligonucleotide-mediated (or site-directed) mutagenesis, PCR mutagenesis, and cassette mutagenesis of an earlier prepared variant or a non-variant version of the antibody. A nucleic acid sequence encoding at least one antibody, portion or polypeptide as described herein can be recombined with vector DNA in accordance with conventional techniques, including but not limited to, blunt-ended or staggered-ended termini for ligation and restriction enzyme digestion. Techniques for such manipulations are disclosed, e.g., by Maniatis et al., Molecular Cloning, Lab. Manual (Cold Spring Harbor Lab. Press, NY, 1982 and 1989), and can be used to construct nucleic acid sequences which encode a monoclonal antibody molecule or antigen-binding region.

In some embodiments, a nucleic acid encoding an antibody or antigen-binding fragment thereof as described herein is comprised by a vector. In some of the aspects described herein, a nucleic acid sequence encoding an antibody or antigen-binding fragment thereof as described herein, or any module thereof, is operably linked to a vector. The term “vector,” as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding an antibody or antigen-binding portion thereof as described herein in place of non-essential viral genes. The vector and/or particle may be utilized for the purpose of transferring any nucleic acids into cells either in vitro or in vivo. Numerous forms of viral vectors are known in the art.

By “recombinant vector,” it is meant that the vector includes a heterologous nucleic acid sequence, or “transgene” that is capable of expression in vivo.

### Pharmaceutical Compositions, Administration and Dosage

The anti-TL1A antibodies provided are useful in a variety of applications including, but not limited to, therapeutic treatment methods, such as the treatment of IBD. The methods of use may be in vitro, ex vivo, or in vivo methods. In certain embodiments, the anti-TL1A antibody is an antagonist for TL1A receptors.

In certain embodiments, the disease treated with anti-TL1A antibody or TL1A receptor antagonist is IBD, CD, UC and/or MR-UC.

In various embodiments, the pharmaceutical compositions are formulated for delivery via any route of administration. “Route of administration” may refer to any administration pathway known in the art, including but not limited to aerosol, nasal, oral, transmucosal, transdermal or parenteral.

“Transdermal” administration may be accomplished using a topical cream or ointment or by means of a transdermal patch.

“Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release.

Via the topical route, the pharmaceutical compositions are formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication.

Via the ocular route, they may be in the form of eye drops.

In various embodiments, an agent can be administered intravenously by injection or by gradual infusion over time. Given an appropriate formulation for a given route, for example, agents useful in the methods and compositions described herein can be administered intravenously, intranasally, by inhalation, intraperitoneally, intramuscularly, subcutaneously, intracavity, and can be delivered by peristaltic means, if desired, or by other means known by those skilled in the art. In particular embodiments, compounds used herein are administered orally, intravenously or intramuscularly to a patient having IBD, CD, UC and/or MR-UC.

The pharmaceutical compositions can also contain any pharmaceutically acceptable carrier. “Pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In various embodiments, provided are pharmaceutical compositions including a pharmaceutically acceptable excipient along with a therapeutically effective amount of an anti-TL1A antibody. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. The active ingredient can be mixed with excipients which are pharmaceutically acceptable and compatible with the active ingredient and in amounts suitable for use in therapeutic methods described herein. Such excipients may be solid, liquid, semisolid, or, in the case of an aerosol composition, gaseous. Suitable excipients are, for example, starch, glucose, lactose, sucrose, gelatin, malt, rice, flour, chalk, silica gel, sodium stearate, glycerol monostearate, talc, sodium chloride, dried skim milk, water, saline, dextrose, propylene glycol, glycerol, ethanol, mannitol, polysorbate or the like and combinations thereof. In addition, if desired, the composition can contain minor amounts of auxiliary substances such as wetting or emulsifying agents, pH buffering agents and the like which enhance or maintain the effectiveness of the active ingredient. Therapeutic compositions as described herein can include pharmaceutically acceptable salts. Pharmaceutically acceptable salts include the acid addition salts formed with inorganic acids such as, for example, hydrochloric or phosphoric acids, organic acids, for example, acetic, tartaric or mandelic, salts formed from inorganic bases such as, for example, sodium, potassium, ammonium, calcium or ferric hydroxides, and salts formed from organic bases such as isopropylamine, trimethylamine, 2-ethylamino ethanol, histidine, procaine and the like. Liquid compositions can contain liquid phases in addition to and in the exclusion of water, for example, glycerin, vegetable oils such as cottonseed oil, and water-oil emulsions. Physiologically tolerable carriers are well known in the art. The amount of an active agent (i.e. antibody or fragment thereof) used that will be effective in the treatment of a particular disorder or condition will depend on the nature of the disorder or condition, and can be determined by one of skill in the art with standard clinical techniques.

The pharmaceutical compositions can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typical dosages of an effective anti-TL1A antibody can be as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of therapeutic method based, for example, on the in vitro responsiveness of the relevant primary cultured cells or histocultured tissue sample, such as biological samples obtained, or the responses observed in the appropriate animal models.

For the treatment of the disease, the appropriate dosage of an antibody depends on the type of disease to be treated, the severity and course of the disease, the responsiveness of the disease, whether the antibody is administered for therapeutic or preventative purposes, previous therapy, and patient's clinical history. The dosage can also be adjusted by the individual physician in the event of any complication and at the discretion of the treating physician. The administering physician can determine optimum dosages, dosing methodologies and repetition rates. The TL1A antibody can be administered one time or over a series of treatments lasting from several days to several months, or until a cure is effected or a diminution of the disease state is achieved (e.g., treatment or amelioration of IBD). The duration of treatment depends upon the subject's clinical progress and responsiveness to therapy. In certain embodiments, dosage is from 0.01 μg to 100 mg per kg of body weight, and can be given once or more daily, weekly, monthly or yearly. For systemic administration, subjects can be administered a therapeutic amount, such as, e.g. about 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more. In certain embodiments, a therapeutic amount is selected from about 1, 3, 10, 30, 100, 300, 600 and 800 milligrams administered as a flat dosage. In certain embodiments, a therapeutic amount is about 1, 2, 3, 4, 5, 6, 7, 8, or 9 milligrams administered as a flat dosage. In certain embodiments, a therapeutic amount is about 10, 20, 30, 40, 50, 60, 70, 80, or 90, milligrams administered as a flat dosage. In certain embodiments, a therapeutic amount is about 100, 150, 200, 250, 300, 350, 400, 450, 500, 550, 600, 650, 700, 750, 800, 850 or 900, milligrams administered as a flat dosage. In certain embodiments, a therapeutic amount is about 5 to about 30 milligrams per kilogram. In certain embodiments, a therapeutic amount is about 5 to about 30 milligrams per kilogram dosed every week or every other week. In certain embodiments, a therapeutic amount is about 5, 10, 15, 20, 25, or 30 milligrams per kilogram. In certain embodiments, a therapeutic amount is about 5, 10, 15, 20, 25, or 30 milligrams per kilogram dosed every week or every other week.

### Methods of Treatment

Various embodiments provide for methods of treating inflammatory bowel disease (IBD), comprising administering an anti-TL1A antibody described herein to a subject in need thereof. In some embodiments, the subject comprises one or more risk genotypes. In some embodiments, the IBD is a severe form of IBD. Severe forms of IBD may be characterized by subclinical phenotypes described herein.

In various embodiments, provided herein is a method of treating inflammatory bowel disease (IBD) in a subject in need thereof, comprising: administering to the subject a therapeutically effective amount of an antibody or an antigen-binding fragment that specifically binds TL1A. In some embodiments, the anti-TL1A antibody comprises a HCFR1 comprising SEQ ID NO: 545, or a sequence that differs from SEQ ID NO: 545 by up to about 5, 4, 3, or 2 amino acids. In some embodiments, the anti-TL1A antibody comprises a HCDR1 selected from SEQ ID NOs: 9,150,484, and 553, or a sequence that differs from a sequence selected from SEQ ID NOs: 9,150,484, and 553 by up to about 5, 4, 3, or 2 amino acids. In some embodiments, the anti-TL1A antibody comprises a HCFR2 comprising SEQ ID NO: 546, or a sequence that differs from SEQ ID NO: 546 by up to about 5, 4, 3, or 2 amino acids. In some embodiments, the anti-TL1A antibody comprises a HCDR2 selected from SEQ ID NOs: 12, 554 to 564 and 574 to 577, or a sequence that differs from SEQ ID NOs: 12, 554 to 564 and 574 to 577 by up to about 5, 4, 3, or 2 amino acids. In some embodiments, the anti-TL1A antibody comprises a HCFR3 comprising a sequence selected from SEQ ID NOs: 547 and 586 to 588, or a sequence that differs from a sequence selected from SEQ ID NOs: 547 and 586 to 588 by up to about 5, 4, 3, or 2 amino acids. In some embodiments, the anti-TL1A antibody comprises a HCDR3 selected from SEQ ID NOs: 15,152,565 to 568, and 578 to 581, or a sequence that differs from a sequence selected from SEQ ID NOs: 15,152,565 to 568, and 578 to 581 by up to about 5, 4, 3, or 2 amino acids. In some embodiments, the anti-TL1A antibody comprises a HCFR4 comprising SEQ ID NO: 548, or a sequence that differs from SEQ ID NO: 548 by up to about 5, 4, 3, or 2 amino acids. In some embodiments, the anti-TL1A antibody comprises a LCFR1 comprising SEQ ID NO: 549, or a sequence that differs from SEQ ID NO: 549 by up to about 5, 4, 3, or 2 amino acids. In some embodiments, the anti-TL1A antibody comprises a LCDR1 selected from SEQ ID NOs: 487, 569 and 570, or a sequence that differs from SEQ ID NOs: 487, 569 and 570 by up to about 5, 4, 3, or 2 amino acids. In some embodiments, the anti-TL1A antibody comprises a LCFR2 comprising SEQ ID NO: 550, or a sequence that differs from SEQ ID NO: 550 by up to about 5, 4, 3, or 2 amino acids. In some embodiments, the anti-TL1A antibody comprises a LCDR2 comprising SEQ ID NO: 488, or a sequence that differs from SEQ ID NO: 488 by up to about 5, 4, 3, or 2 amino acids. In some embodiments, the anti-TL1A antibody comprises a LCFR3 comprising SEQ ID NO: 551, or a sequence that differs from SEQ ID NO: 551 by up to about 5, 4, 3, or 2 amino acids. In some embodiments, the anti-TL1A antibody comprises a LCDR3 selected from SEQ ID NOs: 571 to 573 and 582 to 585, or a sequence that differs from a sequence selected from SEQ ID NOs: 571 to 573 and 582 to 585 by up to about 5, 4, 3, or 2 amino acids. In some embodiments, the anti-TL1A antibody comprises a LCFR4 comprising SEQ ID NO: 552, or a sequence that differs from SEQ ID NO: 552 by up to about 5, 4, 3, or 2 amino acids.

The subject disclosed herein can be a mammal, such as for example a mouse, rat, guinea pig, rabbit, non-human primate, or farm animal. In some instances, the subject is human. In some instances, the subject is a patient who is diagnosed with IBD. In some instances, the subject is not diagnosed with the IBD. In some instances, the subject is suffering from a symptom related to a disease or condition disclosed herein (e.g., abdominal pain, cramping, diarrhea, rectal bleeding, fever, weight loss, fatigue, loss of appetite, dehydration, and malnutrition, anemia, or ulcers).

In various embodiments, the subject is not responsive to induction of an anti-TNF therapy (e.g., adalimumab, certolizumab, ctanercept, golimumab, infliximab) (anti-TNF non-response), or loses response to said anti-TNF therapy after a period of time during treatment (anti-TNF loss of response). In various embodiments, the subject is at risk for developing anti-TNF non-response or anti-TNF loss of response. In some embodiments, the subject is treated by administering the anti-TL1A antibody disclosed herein to the subject, provided the subject is at risk for developing, or suffers from, anti-TNF non-response or anti-TNF loss of response.

In various other embodiments, the subject is determined to have an increased TL1A expression. In some embodiments, the administration of a therapeutically effective amount of an anti-TL1A antibody causes a decrease in TL1A in the subject treated.

Methods disclosed herein provide methods of treating an inflammatory bowel disease (IBD) in a subject by administering an anti-TL1A antibody described herein to the subject. In various embodiments, IBD is Crohn's Disease (CD) or ulcerative colitis (UC). In some embodiments, the IBD is a severe form of IBD. In some embodiments, the severe form of IBD is characterized by a subclinical phenotype. In some embodiments, the IBD is a moderate to severe form of IBD. In some embodiments, the IBD is a moderate form of IBD.

Subclinical phenotypes of IBD may include, but are not limited to, non-stricturing, stricturing, stricturing and penetrating, and isolated internal penetrating, disease, and perianal CD (pCD). Stricturing is the progressive narrowing of the intestine. Internal penetrating disease creates abnormal passageways (fistulac) between the bowel and other structures. pCD is a form of Crohn's disease that causes inflammation around the anus.

The IBD may be refractory. The term “medically refractory,” or “refractory,” as used herein, refers to the failure of a standard treatment to induce remission of a disease. In some embodiments, the disease comprises an inflammatory disease disclosed herein. A non-limiting example of refractory inflammatory disease includes refractory Crohn's disease, and refractory ulcerative colitis (e.g., mrUC). Non-limiting examples of standard treatment include glucocorticosteriods, anti-TNF therapy, anti-α4-b7 therapy (vedolizumab), anti-IL12p40 therapy (ustekinumab), Thalidomide, and Cytoxin. In some embodiments, the UC is medically refractory UC (mrUC). In some embodiments, the CD is refractory.

Disclosed herein are methods of administering the anti-TL1A antibody to a subject in need thereof. In various embodiments, the antibody is a monoclonal antibody. In some embodiments, the antibody is a human antibody. In various embodiments, the antibody is a humanized antibody. In various embodiments, the antibody is a neutralizing antibody.

In various aspects, the anti-TL1A antibody is administered to the subject for treatment of an IBD described herein. In various other embodiments, the anti-TL1A antibody is administered in a series of treatments. In some embodiments, the anti-TL1A antibody and a second IBD treatment may be administered in any order or concurrently. In selected embodiments, the anti-TL1A antibody will be administered to patients that have previously undergone treatment with the second IBD treatment. In certain other embodiments, the anti-TL1A antibody and the second IBD treatment will be administered substantially simultaneously or concurrently. For example, a subject may be given the anti-TL1A antibody while undergoing a course of treatment with the second IBD treatment. In certain embodiments, the anti-TL1A antibody will be administered within 1 year of the treatment with the second IBD treatment. In certain alternative embodiments, the anti-TL1A antibody will be administered within 10, 8, 6, 4, or 2 months of any treatment with the second IBD treatment. In certain other embodiments, the anti-TL1A antibody will be administered within 4, 3, 2, or 1 week of any treatment with the second IBD treatment. In some embodiments, the anti-TL1A antibody will be administered within 5, 4, 3, 2, or 1 days of any treatment with the second IBD treatment. It will further be appreciated that the two treatments may be administered to the subject within a matter of hours or minutes (i.e., simultaneously).

Other IBD treatments include, but are not limited to 1) anti-inflammatory drugs (e.g., Aminosalicylates such as, but not limited to sulfasalazine Azulfidine, 5-aminosalicylates, Mesalamine, Asacol, Lialda, Rowasa, Canasa, balsalazide Colazal and olsalazine, Dipentum); 2) corticosteroids (e.g., prednisone and hydrocortisone); 3) immune system suppressors (e.g., Azathioprine, Azasan, Imuran, mercaptopurine, Purinethol, Purixan, Cyclosporine, Gengraf, Neoral and Sandimmune, Infliximab, Remicade, adalimumab, Humira, golimumab, and Simponi, tumor necrosis factor (TNF)-alpha inhibitors (e.g., Infliximab), Methotrexate, Rheumatrex, Natalizumab, Tysabri, vedolizumab, Entyvio, Ustekinumab and Stelara; 4) Antibiotics (e.g., Metronidazole, Flagyl, Ciprofloxacin, Cipro); 5) Anti-diarrheal medications (e.g., fiber supplements-Metamucil or Citrucel) or loperamide; 6) Pain relievers (e.g., Tylenol, ibuprofen, naproxen sodium and diclofenac sodium); and 7) Surgery (e.g., removal of the colon, partial digestive tract removal, colectomy, proctocolectomy and/or strictureplasty). In some embodiments, these IBD treatments may be administered in combination with the anti-TL1A antibody. Treatment with an antibody can occur prior to, concurrently with, or subsequent to administration of an IBD treatment. Combined administration can include co-administration, either in a single pharmaceutical formulation or using separate formulations, or consecutive administration in either order but generally within a time period such that all active agents can exert their biological activities simultaneously. Any dosing schedules for such IBD treatments can also be used as determined by the skilled practitioner.

In some embodiments, the second IBD treatment comprises an antibody. Thus, treatment can involve the combined administration of antibodies provided herein with other antibodies against additional IBD-associated antigens, such as, but not limited to tumor necrosis factor (TNF)-alpha. Combined administration can include co-administration, either in a single pharmaceutical formulation or using separate formulations, or consecutive administration in either order but generally within a time period such that all active agents can exert their biological activities simultaneously.

### Kits

Further provided is a kit to treat IBD (e.g., CD, UC and/or mrUC). The kit comprises of the antibodies described herein, which can be used to perform the methods described herein. The kit is useful for practicing the inventive method of providing treatment to an IBD, CD, UC and/or mrUC patient by administering an anti-TL1A antibody. The kit is an assemblage of materials or components, including at least one of the inventive compositions. Thus, in some embodiments, the kit contains a composition including anti-TL1A antibodies, for the treatment of IBD, CD, UC and/or MR-UC, as described above. In other embodiments, the kits contain all of the components necessary and/or sufficient to perform a detection assay for TL1A, including all controls, directions for performing assays, and any necessary software for analysis and presentation of results.

The exact nature of the components configured in the inventive kit depends on its intended purpose. For example, some embodiments are configured for the purpose of treating IBD, CD, UC and/or MR-UC. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat or alleviate IBD, CD, UC and/or MR-UC. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of an inventive composition containing anti-TL1A antibodies and/or primers and probes for TL1A. The packaging material generally has an external label which indicates the contents and/or purpose of the kit and/or its components.

### EXAMPLES

The following examples are illustrative of the embodiments described herein and are not to be interpreted as limiting the scope of this disclosure. To the extent that specific materials are mentioned, it is merely for purposes of illustration and is not intended to be limiting. One skilled in the art may develop equivalent means or reactants without the exercise of inventive capacity and without departing from the scope of this disclosure.

### Example 1: Generation and Characterization of Humanized Anti-TL1A Antibodies

A murine anti-TL1A antibody was humanized to reduce potential immunogenicity. A first variant, 12835, was generated, which consists of murine 5C3D11 CDRs (SEQ ID NOs: 9, 12, 15, 18, 21, 24) grafted into human variable region frameworks to generate a heavy chain variable region comprising SEQ ID NO: 26 and a light chain variable region comprising SEQ ID NO: 28. Unfortunately, clone 12835 contains nine (9) framework back mutations (murine framework residues), resulting in an incompletely humanized variant. Complete humanization is important to reduce the chances of a subject raising an immune response to an administered antibody. Consequently, the goal was to generate humanized antibodies comprising fewer murine framework residues while retaining the functional activity of the parent 12835 antibody. Unfortunately, using visual inspection of the sequence it is not straight-forward to distinguish murine framework residues critical for the antibody's function from those that are non-critical, and thus which amino acid residues can be replaced by the corresponding human framework residue. Therefore, as a first step, 12835 was rehumanized by CDR grafting into the closest fully human germline frameworks (IGV1-46*02 and IGKV3-20*01 as determined by the NCBI's igblast tool). This clone is L8 and comprises the 5C3D11 and 12835 CDRs as defined by the combination of the Kabat, Chothia, and IMGT methods (HCDR1, GFDIQDTYMH; HCDR2, RIDPASGHTKYDPKFQV; HCDR3, SRSGGLPDV; LCDR1, RASSSVSYMY; LCDR2, ATSNLAS; LCDR3, QQWSGNPRT).

In the present study, many variants of 12835 were made and tested in order to identify a more human-like antibody that retains the functional activity of the parent 12835 antibody. In the first stage, variants containing significantly fewer murine framework residues were identified. Subsequently, CDR libraries of 12835 were combined with a fully human germline frameworks in order to identify multiple variants that do not contain any murine framework residues, yet retain the functional activity and/or affinity of the parent 12835 antibody.

Cloning of Murine 5C3D11 and Humanized Construct 12835 into Phage Expression System

DNA encoding the heavy and light chain variable regions of both murine 5C3D11 and humanized 12835 was cloned into a phage expression vector that contained human kappa light chain constant domain and human Gl heavy chain constant domain 1. In addition, the vector contains a his-tag and hemagglutinin A tag at the carboxy-terminal end of the heavy chain to facilitate purification and detection. Cloning of the murine variable regions into the phage expression vector containing human constant domains results in the expression of chimeric 5C3D11.

The murine 5C3D11 heavy chain variable region DNA (SEQ ID NO: 1) and light chain variable region DNA (SEQ ID NO: 4) were codon optimized for bacterial expression to generate SEQ ID NOS: 2 and 5, respectively. The humanized 12835 heavy chain variable region DNA was codon optimized to generate SEQ ID NO: 25 and the light chain variable region DNA was codon optimized to generate SEQ ID NO: 27.

Expression and Quantitation of Fab in the Periplasmic Space of E. coli.

Cloning was verified by expressing and quantitating Fab in the periplasmic space of E. coli. Briefly, XL-O bacteria were grown in 2X YT medium at 37° C. until the culture reached a density of 0.9-1.1 at OD600. Isopropyl β-D-thiogalactoside was then added to the cells to a final concentration of 1 mM and 3.0 mL of culture was transferred to a 14 mL snap-top tube. Each tube was transfected with 25 μL of high titer phage stock and the cultures were placed in a shaker (225 rpm) at 37° C. One hour later, the temperature was shifted to 25° C. and the cultures were grown for an additional 14-16 h. The cells were collected by centrifugation at 3900 rpm for 30 min in an Eppendorf 5810R centrifuge (˜3,200×g), the supernatant was decanted and the cells were resuspended in 0.3 mL of lysis buffer (30 mM Tris, pH 8.0, 2 mM EDTA, 20% sucrose, 2 mg/ml lysozyme, 5 U/mL DNase I) and placed on ice for 15 min. The cell suspension was transferred to a 1.5 mL tube and cell debris was pelleted by centrifugation at 15,000 rpm for 15 min in an Eppendorf 5424 microfuge (˜21,000×g). The supernatant was removed carefully without disturbing the pellet and was stored at 4° C. until usc.

In order to quantitate Fab expression, a 96-well Costar-3366 plate was coated with 50 μl/well of 2 μg/ml sheep anti-human Fd (Southern Biotech, Prod. #2046-01, Lot #A7212-VJ06) in PBS overnight at 4° C. The plate was washed three times with PBS containing 0.05% Tween 20 (PBS-T) and 50 μl/well of sample dilutions was added. Sample dilutions were performed with PBS-T. A standard curve was generated using human Fab (Rockland, Prod. #009-01015, Lot #38543) diluted serially 3-fold, beginning at 500 ng/ml. The plates were incubated 1 h at 25° C., washed three times with PBS-T, and incubated with 50 μl/well of anti-kappa HRP conjugate (Southern Biotech, Prod. #2060-05, Lot #K3114-S506B), diluted 10,000-fold in PBS-T for 1 h at 25° C. The plate was washed three times with PBS-T, developed with 50 μl/well 1-Step Ultra TMB-ELISA (Thermo Scientific, Prod. #34028, Lot #SF2405221). The reaction was terminated by the addition of 2 N H₂SO₄ and the A650 and A450 were determined before and after addition of H₂SO₄, respectively, using a Spectramax plate reader.

### Characterization of Chimeric 5C3D11 and 12835-Filter Lift Assay

A filter lift assay was developed to facilitate characterization of heavy and light chain expression and to verify functional activity of the Fab constructs through binding to biotinylated antigen. With filter lift assays bacterial lawns are infected with phage under conditions where each phage produces a distinct plaque (zone of slower growing bacteria). Nitrocellulose filters are placed on the lawns, capturing expressed Fab. Subsequently, the filters can be probed with biotinylated antigen and/or reagents directed against immunoglobulins or peptide tags.

Dilutions of high titer phage stocks (typically 106-fold) were combined with 0.35 ml of a confluent E. coli strain XL culture and 20 μg/ml tetracycline. The mixture was combined with 3.5 ml top agar (0.7% Bacto-agar in Luria broth) and overlaid on an LB agar plate (1.5% Bacto-agar in Luria broth). The plate was incubated 6-8 h at 37° C. at which time a nitrocellulose filter (Whatman 82-mm diameter, 0.45 μm pore size, GE Healthcare, Prod. #10401116) was placed on top and the plate is incubated at 25° C. for 12-15 h. The filter was removed, rinsed briefly in PBS, and transferred to 5% M-P blocking solution for 2 h at 25° C. with constant agitation.

Subsequently, the filter was cut into three sections: one to assess light chain expression, one to assess heavy chain expression, and one to assess antigen binding. Each section was transferred to the primary detection reagent: goat anti-human kappa, HRP conjugate (Southern Biotech, Prod. #2060-05, Lot #K3114-S506B) diluted 1000-fold in 5% M-P for detection of light chain, rat anti-HA, HRP conjugate (Roche, Prod. #12013819001) diluted 1000-fold in 5% M-P for detection of heavy chain, or biotinylated antigen at the desired concentration in 5% M-P.

In order to label antigen with biotin, 500 μg of human TL1A (Fitzgerald, Prod. #30R-AT070, Lot #A13102302) was resuspended in water to 1 mg/ml. Following suspension in water the protein was in 10 mM Tris, pH 8.5 with 75 mM arginine. The Tris and arginine were removed by buffer exchange using a 7K MW cut-off, 5 ml Zeba spin desalting column (Thermo Prod. #89891) that had been equilibrated with 10 mM phosphate buffer, pH 8.0 with 65 mM NaCl. After recovering the protein, it was immediately biotinylated by combining it with EZ-Link Sulfo-NHS-LC-Biotin (Thermo Prod. #21327) at a 5:1 molar at 25° C. for 30 min. The reaction was terminated by the addition of 750 mM arginine to achieve a final concentration of 75 mM. The reaction was transferred to ice and stored at 4° C.

Filters were incubated 2 h at 25° C. with constant agitation, washed 5 times in PBS-0.05% Tween 20 (2 minutes each wash with constant agitation). The filters probed with biotinylated antigen were transferred to 10 ml of High Sensitivity Neutravidin, HRP conjugate (Thermo Scientific, Prod. #31030) diluted 5000-fold in 1% BSA in PBS and incubated for 1 h at 25° C. The filters were then washed 5 times in PBS-0.05% Tween 20 (2 minutes each wash with constant agitation). All filters were developed with 1-Step Ultra TMB-Blotting (Thermo Scientific, Prod. #37574).

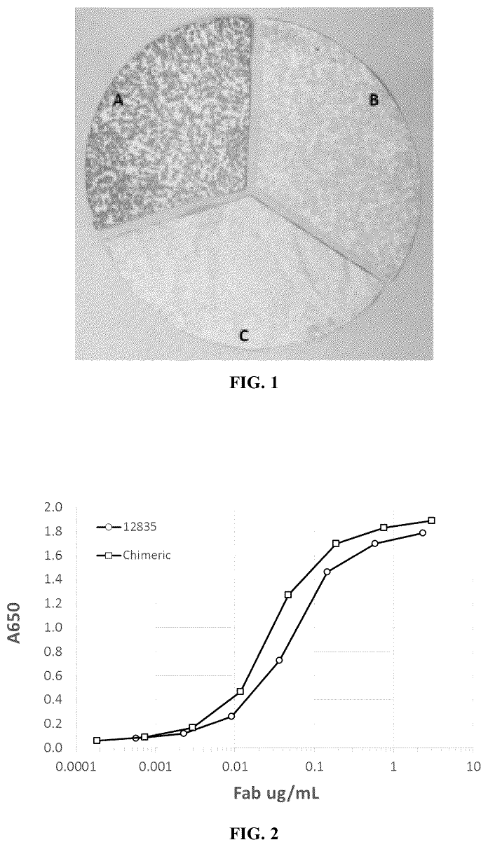

Using this approach, expression of heavy chain (FIG. 1A) and light chain (FIG. 1B) was demonstrated. Furthermore, when the filter was probed with 8 nM biotinylated human TL1A staining was observed (FIG. 1C) indicating the bacteria were expressing functional Fab.

### Characterization of Fab Binding by ELISA

The filter lift assay provides a qualitative assessment of antigen binding. In order to compare the binding activity of chimeric 5C3D11 with the humanized construct 12835 in a more quantitative fashion an ELISA was developed. A 96-well Costar-3366 plate was coated with 50 μl/well of 2 μg/ml human TL1A (Fitzgerald, Prod. #30R-AT070, Lot #A13102302) in PBS overnight at 4° C. The plate was rinsed once with PBS-T and blocked with 100 μl/well of 1% BSA in PBS (1% BSA) for 1 h at 25° C. Fab samples were serially diluted 3-fold using 1% BSA and were incubated for 1 h at 25° C. (50 μl/well). The plate was washed three times with PBS-T and 50 μl/well anti-human kappa, HRP conjugate (Southern Biotech, Prod. #2060-05, Lot #K3114-S506B) diluted 10,000-fold in 1% BSA was added for 1 h at 25° C. In certain assays (extended wash format), the plate was placed in large volumes (up to 1L) of PBS-T and incubated, with mixing, for 2-5 hours prior to the addition of anti-human kappa, HRP conjugate. The plate was washed three times with PBS-T, developed with 50 μl/well 1-Step Ultra TMB-ELISA (Thermo Scientific, Prod. #34028, Lot #SF2405221). The reaction was terminated by the addition of 2 N H2SO4 and the A650 and A450 were determined before and after addition of H₂SO₄, respectively, using a Spectramax plate reader. For measuring binding to murine TL1A the same protocol was used, but the plates were coated with 2 μg/ml murine TL1A (BioLegend, Prod. #753004, Lot #B204691) and the Fab samples were serially diluted 2-fold.

The binding activity of chimeric 5C3D11 Fab was compared to humanized 12835 Fab (FIG. 2). Although the binding activities of 5C3D11 and humanized 12835 in an IgG format (bivalent) appeared similar, it was observed that the binding activity of 12835 in the Fab format (monovalent) was somewhat diminished compared to chimeric Fab (FIG. 2). It is likely that this discrepancy reflects differences in true affinity (monovalent format) versus similar avidities (bivalent format). Using the monovalent assay format, the chimeric Fab appears to be 2- to 3-fold higher affinity than humanized 12835 Fab.

### Capture Lift Assay

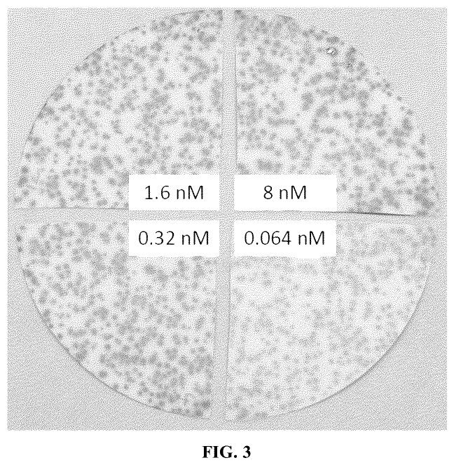

A nitrocellulose filter (Whatman 82-mm diameter, 0.45 μm pore size, GE Healthcare, Prod. #10401116) was floated on top of 10 ml of 10 mg/ml goat anti-human kappa (Southern Biotech Prod. #2060-01) for 2 h at 25° C. The filter was submersed briefly before removing and transferred to 10 ml of 5% M-P for 2 h at 25° C. The filter was removed from 5% M-P, rinsed briefly one time with PBS, and was air-dried. Subsequently, the filter was processed in the same way as the filter lift assay described above, with minor modifications. Briefly, dilutions of high titer phage stocks (typically 106-fold) were combined with 0.35 ml of a confluent E. coli strain XL culture and 20 μg/ml tetracycline. The mixture was combined with 3.5 ml top agar (0.7% Bacto-agar in Luria broth) and overlaid on an LB agar plate (1.5% Bacto-agar in Luria broth). The plate was incubated 6-8 h at 37° C. at which time the pre-treated nitrocellulose filter (described above) was placed on top and the plate was incubated for 12-15 h at 25° C. The filter was removed, rinsed briefly in PBS, and transferred to biotinylated antigen at the desired concentration in 5% M-P. Filters were incubated 2 h at 25° C. with constant agitation, washed 5 times in PBS-0.05% Tween 20 (2 minutes each wash with constant agitation) and were transferred to 10 ml of High Sensitivity Neutravidin, HRP conjugate (Thermo Scientific, Prod. #31030) diluted 5000-fold in 1% BSA in PBS and incubated for 1 h at 25° C. The filters were then washed 5 times in PBS-0.05% Tween 20 (2 minutes each wash with constant agitation). All filters were developed with 1-Step Ultra TMB-Blotting (Thermo Scientific, Prod. #37574). The developed filter as shown in FIG. 3 demonstrates high sensitivity and avidity of 5C3D11 to TL1A.

Removal of Murine Framework Residues from 12835 to Identify Multiple Active Humanized Clones, Including 18-7 and 21-3

Murine framework residues were removed using Kunkel mutagenesis (Kunkel TA 1985. PNAS 82:488-492). Briefly, single strand M13 plasmid was isolated and primed for DNA replication with mutagenic oligonucleotide(s) encoding the human instead of the murine framework residue. After extension to complete the circle, transformation of bacteria resulted in a mixture of wild type (murine framework residue unmated) and mutated (human framework residue) plasmids. Mutagenesis was performed at multiple sites simultaneously to generate small combinatorial libraries containing mixtures of clones containing various combinations of murine and human framework mutations. Subsequently, the mixtures were plated and screened by capture lift to identify the most active framework combinations. Library clones were characterized by DNA sequencing.

Fab was expressed in E. coli, quantitated by ELISA, and binding activity was assessed by ELISA by titrating against immobilized antigen. Expression of Fab, isolation of the periplasmic fraction, quantitation of Fab expression and binding to antigen by ELISA were all performed as described above.

Using this approach, multiple active clones containing differing numbers of murine framework residues were identified. Examples of active clones with varying numbers and positions of murine framework residues are summarized in Table 3.

TABLE 3
Active humanized 5C3D11 constructs with varying amounts of framework back mutations

| | | | | | | | | | | Mu FR Back |
|---|---|---|---|---|---|---|---|---|---|---|
| Clone | Light Chain | | | | | | Heavy Chain | | | Mutations |
| 12835 | V19 | M21 | P46 | W47 | V58 | Y71 | L20 | T71 | S93 | 9 |
| 1-3 | | | | | V58I | | | | | 8 |
| 5-2 | | | | | | | | T71R | | 8 |
| 22 | | | | | | Y71F | | | | 8 |
| 5-4 | V19A | M21L | | | | | | | | 7 |
| 1-4 | | | P46L | | V58I | | | | | 7 |
| 2-3 | V19A | M21L | | | V58I | | | | | 6 |
| 3-4 | V19A | M21L | | | | Y71F | | | | 6 |
| 5-1 | V19A | M21L | | | | | | T71R | | 6 |
| 7-4 | V19A | M21L | P46L | | | | | | | 6 |
| 26 | V19A | M21L | | | | | L20V | | | 6 |
| 9-1 | V19A | M21L | | | | Y71F | | T71R | | 5 |
| 13-2 | V19A | M21L | | | | | | T71R | S93A | 5 |
| 21 | V19A | M21L | P46L | | V58I | | | | | 5 |
| 27 | V19A | M21L | | W47L | | | L20V | | | 5 |
| 10-1 | V19A | M21L | P46L | W47L | | | | T71R | | 4 |
| 19 | V19A | M21L | P46L | W47L | V58I | | | | | 4 |
| 16-2 | V19A | M21L | P46L | W47L | V58I | | | T71R | | 3 |
| 17-2 | V19A | M21L | P46L | W47L | | Y71F | | T71R | | 3 |
| 18-7 | V19A | M21L | P46L | W47L | V581 | Y71F | | T71R | | 2 |
| 19-5 | V19A | M21L | P46L | W47L | | | L20V | T71R | | 3 |
| 20-7 | V19A | M21L | P46L | W47L | | | | T71R | S93A | 3 |
| 21-3 | V19A | M21L | P46L | W47L | | | L20V | T71R | S93A | 2 |

### Synthesis of CDR-Grafted Construct

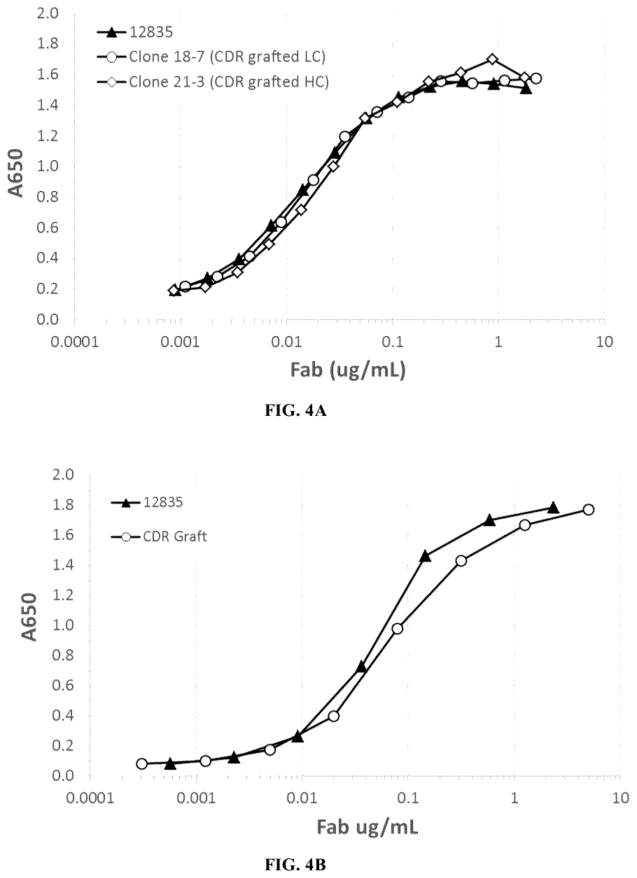

Two of the clones identified, 18-7 and 21-3, contained only two murine framework back mutations. The light chain of clone 18-7 did not contain any murine framework residues while the heavy chain of clone 21-3 did not contain any murine framework residues. Screening of the framework combinatorial libraries did not identify a CDR-grafted variant (no murine framework residues on both the heavy and light chains). For comparison, the CDR-grafted variant was synthesized using Kunkel mutagenesis and compared its binding activity was characterized by ELISA. Although the CDR grafted construct bound antigen the humanized 12835 variant consistently displayed stronger binding to antigen (FIG. 4B).

After making the back mutations of murine framework residues as indicated in Table 3, the heavy chain variable region frameworks 1-3 were identical to human germlines IGHV1-46*01, IGHV1-46*02, and IGHV1-46*03 while the light chain variable region frameworks were identical to human germline IGKV3-20*01.

In addition, different back-mutations were introduced into the third framework of the heavy chain variable region such that the new heavy chain variable regions were homologous to human germline IGHV1-3*01 (see VH SEQ IDs 519, 521, 523, 525, 527, 529, 531, 533, 535, 537, 539, and 541). Collectively, clones containing these back-mutations are referred to as alternative framework variants.

### Characterization of Humanized Variants in an Alternative ELISA Format

Multiple Fab variants were characterized by ELISA, using an alternative format that permits the rapid and direct comparison (single-well determination with no dilution series required) of the relative affinities of different Fab clones isolated from different cultures, regardless of the relative expression levels of the clones (Watkins et al. 1997, Analytical Biochemistry 253). This assay enables a more quantitative comparison of the relative binding strengths of the variants as the plate is saturated with the different Fabs despite their different expression levels. Thus, slight differences in binding profiles caused by variation in the Fab quantitation assay are eliminated. Briefly, a 96-well Costar-3366 plate was coated with 50 μl/well of 2 μg/ml goat anti-human kappa (Southern Biotech Prod. #2060-01) for 2 h at 25° C., washed once with PBS-0.05% Tween 20, and incubated with 50 μl/well of sample Fab for 2 h at 25° C. The plate was washed 4 times with PBS-0.05% Tween 20 and incubated with 50 μl/well serial dilutions of biotinylated antigen for 2 h at 25° C. Preparation of biotinylated antigen was described above. The plate was washed 4 times with PBS-0.05% Tween 20 and incubated with 50 μl/well of high sensitivity Neutravidin, HRP conjugate (Thermo Scientific, Prod. #31030) diluted 5000-fold in 1% BSA in PBS for 1 h at 25° C. The plate was washed three times with PBS-T, developed with 50 μl/well 1-Step Ultra TMB-ELISA (Thermo Scientific, Prod. #34028, Lot #SF2405221). The reaction was terminated by the addition of 2 N H2SO4 and the A650 and A450 were determined before and after addition of H2SO4, respectively, using a Spectramax plate reader.

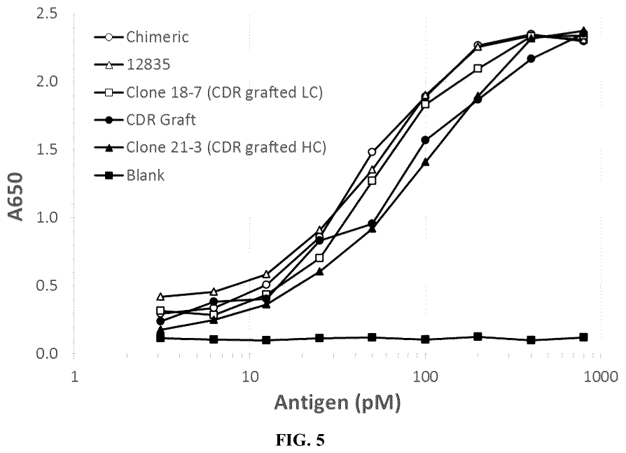

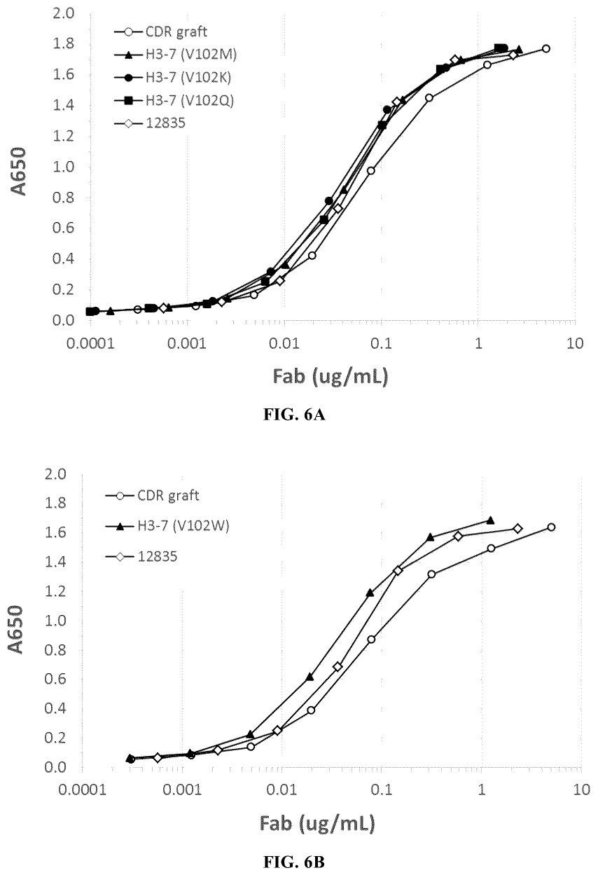

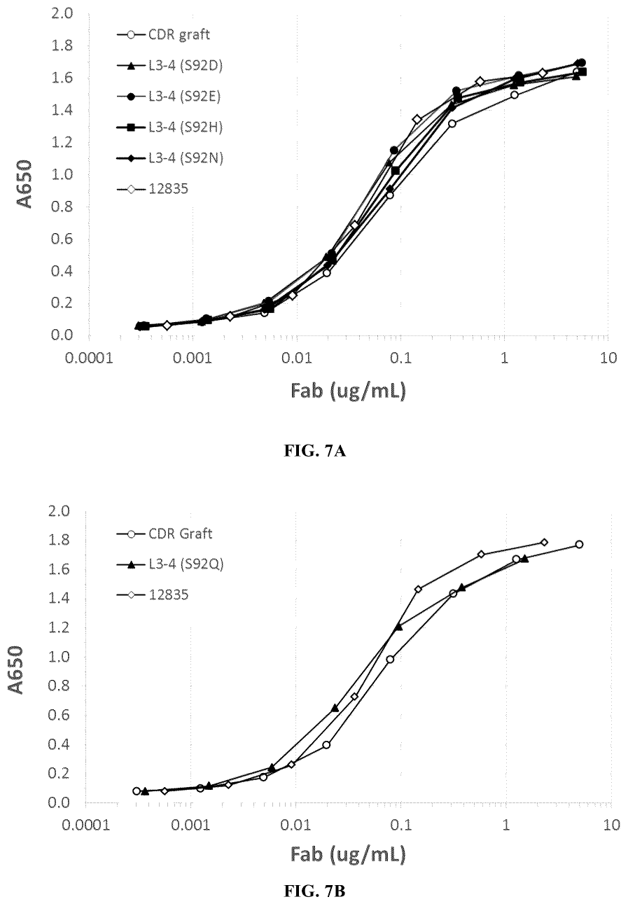

The chimeric Fab binds antigen more strongly than the CDR grafted Fab in the alternative ELISA format (FIG. 5, compare open circles with closed circles). The humanized 12835 clone has slightly diminished binding compared to the chimeric (compare open triangles with open circles), followed by clone 18-7 and 21-3. The binding of clone 21-3 was most similar to the CDR-grafted variant, suggesting that one of the murine heavy chain framework back mutations may be important for maintaining full binding activity with the parental (wild-type) CDRs.

### Example 2: Generation and Characterization of Anti-TL1A Antibodies Having Optimized CDRs

In order to identify CDR mutations that could restore and improve the binding activity of the CDR grafted construct (fully human, germline frameworks) each position of all six CDRs (LCDR1, LCDR2, LCDR3, HCDR1, HCDR2, and HCDR3) was mutagenized individually by Kunkel mutagenesis using degenerate oligonucleotides in which the codon encoding the target amino acid was replaced with NNK. Initially, one library was synthesized at each position of HCDR3 and LCDR3. These positional libraries, with a theoretical diversity of 32 codons/20 amino acids/1 stop codon, were screened by capture lift. In some cases, each position was screened by itself (theoretical diversity of library equals 32) while in other cases the positions of a particular CDR were pooled and screened as a CDR library (theoretical diversity of library equals 32 times the number of positions pooled; e.g. HCDR3 consists of 7 positions, so the theoretical library size was 32×7 equals 224 members). The libraries were screened at concentrations of biotinylated human TL1A ranging from 15 to 1,000 pM. Positive plaques were picked and sequenced. Fab was expressed, isolated from periplasmic fraction, and characterized by ELISA, as described previously. Summaries of the capture lift screening, DNA sequencing, and relative binding activities by ELISA for some of the initial screens of LCDR3 and HCDR3 are shown in Tables 4 and 5, respectively. The results of more exhaustive capture lift screening of all six CDRs are summarized in Tables 6 to 12.

In addition, LCDR3 and HCDR3 libraries constructed using an alternative heavy chain variable region framework were made and screened by capture lift (Tables 13 and 14). Screening CDR libraries constructed on the alternative framework identified some mutations that were identified on the VH1-46*01 framework, but also identified novel mutations not previously identified (heavy chain CDR3 L98S, V102H, V102F and light chain CDR3 S92A, S92F, and S92Y, for example).

TABLE 4
Light Chain CDR3 Positional Scan (screened with 200 pM antigen)

| | | | | | | | Relative ELISA |
|---|---|---|---|---|---|---|---|
| | | | | | | | Activity |
| | Amino | | CL | CL | SEQS | Mutations | (Strongest to |
| Pos. | Acid | Plaques | Hits | Picks | Acquired | (frequency) | weakest binders) |
| 1 | Q89 | 374 | 19 | 12 | 10 | H (3) | Q = N > H, S |
| | | | | | | N (3) | |
| | | | | | | S (2) | |
| | | | | | | Q (2) | |
| 2 | Q90 | 322 | 4 | 4 | 2 | Q (2) | |
| 3 | W91 | 234 | 0 | 6 | 1 | S (1) | W >>> S (inactive) |
| | | | | (random) | | | |
| 4 | S92 | 212 | 44 | 12 | 11 | E (4) | D,E > H, N, Q > S |
| | | | | | | D (2) | |
| | | | | | | Q (2) | |
| | | | | | | N (1) | |
| | | | | | | V (1) | |
| | | | | | | H (1) | |
| 5 | G93 | 168 | 22 | 12 | 11 | S (3) | G = A > D > Q, S |
| | | | | | | A (1) | |
| | | | | | | D (1) | |
| | | | | | | Q (1) | |
| | | | | | | G (5) | |
| 6 | N94 | 224 | 2 | 2 | 1 | N (1) | |
| 7 | P95 | 160 | 10 | 10 | 10 | P (10) | |
| 8 | R96 | 202 | 12 | 12 | 9 | R (9) | |
| 9 | T97 | 662 | 108 | 12 | 12 | S (4) | T > S |

TABLE 5
Heavy Chain CDR3 Positional Scan (screened with 500 pM antigen)

| | | | | | | | Relative ELISA |
|---|---|---|---|---|---|---|---|
| | Amino | | CL | CL | SEQS | Mutations | Activity (Strongest to |
| Pos. | Acid | Plaques | Hits | Picks | Acquired | (frequency) | weakest binders) |
| 1 | S95 | 228 | 29 | 12 | 12 | L (1) | S >>> L (inactive) |
| | | | | | | S (11) | |
| 2 | G96 | 272 | 6 | 6 | 5 | A (1) | G > A |
| | | | | | | G (4) | |
| 3 | G97 | 188 | 7 | 7 | 7 | G (7) | |
| 4 | L98 | 192 | 23 | 12 | 10 | M (2) | L = M > A |
| | | | | | | A (2) | |
| | | | | | | L (6) | |
| 5 | P99 | 396 | 58 | 12 | 11 | P (11) | |
| 6 | D101 | 165 | 2 | 2 | 1 | E (1) | D > E |
| 7 | V102 | >300 | 57 | 12 | 12 | M (5) | M, K, Q, W > V = T |
| | | | | | | K (2) | |
| | | | | | | R (1) | |
| | | | | | | S (1) | |
| | | | | | | T (1) | |
| | | | | | | Q (1) | |
| | | | | | | W (1) | |

TABLE 6
Heavy Chain CDR1 Screening Summary (L8 template; 1-46*02 framework)

| [b-TL1A] | 26 | 27 | 28 | 29 | 30 | 31 | 32 | 33 | 34 | 35 | |
|---|---|---|---|---|---|---|---|---|---|---|---|
| (pM) | G | F | D | I | Q | D | T | Y | M | H | CDR SEQ |
| 500 | G | F | E | I | Q | D | T | Y | M | H | GFEIQDTYMH |
| 500 | G | F | D | P | Q | D | T | Y | M | H | GFDPQDTYMH |
| 500 | G | F | D | V | Q | D | T | Y | M | H | GFDVQDTYMH |
| 500 | G | F | D | I | G | D | T | Y | M | H | GFDIGDTYMH |
| 500 | G | F | D | I | S | D | T | Y | M | H | GFDISDTYMH |
| 500 | G | F | D | I | V | D | T | Y | M | H | GFDIVDTYMH |
| 500 | G | F | D | I | Q | D | A | Y | M | H | GFDIQDAYMH |
| 500 | G | F | D | I | Q | D | S | Y | M | H | GFDIQDSYMH |
| 500 | G | F | D | I | Q | D | T | F | M | H | GFDIQDTFMH |
| 500 | G | F | D | I | Q | D | T | Y | I | H | GFDIQDTYIH |
| 150 | G | F | D | L | Q | D | T | Y | M | H | GFDLQDTYMH |
| 150 | G | F | D | P | Q | D | T | Y | M | H | GFDPQDTYMH |
| 150 | G | F | D | I | S | D | T | Y | M | H | GFDISDTYMH |
| 150 | G | F | D | I | Q | D | T | Y | I | H | GFDIQDTYIH |
| 150 | G | F | D | I | Q | D | T | Y | L | H | GFDIQDTYLH |

TABLE 7
Heavy Chain CDR2a Screening Summary
(L8 template; 1-46*02 framework)

| [b- | | | | | | | | | | | |
|---|---|---|---|---|---|---|---|---|---|---|---|
| TL1A] | 50 | 51 | 52 | 52a | 53 | 54 | 55 | 56 | 57 | 58 | |
| (pM) | R | I | D | P | A | S | G | H | T | K | CDR SEQ |
| 200 | R | L | D | P | A | S | G | H | T | K | RLDPASGHTK |
| 200 | R | I | E | P | A | S | G | H | T | K | RIEPASGHTK |
| 200 | R | I | D | P | E | S | G | H | T | K | RIDPESGHTK |
| 200 | R | I | D | P | A | S | G | H | T | K | RIDPASGHTK |
| 200 | R | I | D | P | A | G | G | H | T | K | RIDPAGGHTK |
| 200 | R | I | D | P | A | S | A | H | T | K | RIDPASAHTK |
| 200 | R | I | D | P | A | S | G | H | I | K | RIDPASGHIK |
| 200 | R | I | D | P | A | S | G | H | L | K | RIDPASGHLK |
| 200 | R | I | D | P | A | S | G | H | V | K | RIDPASGHVK |

TABLE 8
Heavy Chain CDR2b Screening Summary (various templates; 1-46*02
framework)

| [b-TL1A] | 59 | 60 | 61 | 62 | 63 | 64 | 65 | | |
|---|---|---|---|---|---|---|---|---|---|
| (pM) | Y | D | P | K | F | Q | V | CDR SEQ | Template |
| 100 | I | D | P | K | F | Q | V | IDPKFQV | 17V |
| 100 | L | D | P | K | F | Q | V | LDPKFQV | 20L |
| 100 | M | D | P | K | F | Q | V | MDPKFQV | 20EL |
| 100 | S | D | P | K | F | Q | V | SDPKFQV | 20L |
| 100 | T | D | P | K | F | Q | V | TDPKFQV | 20L |
| 100 | V | D | P | K | F | Q | V | VDPKFQV | 20EL |
| 100 | Y | I | P | K | F | Q | V | YIPKFQV | 20EL |
| 100 | Y | N | P | K | F | Q | V | YNPKFQV | 17V, 20EL |
| 100 | Y | R | P | K | F | Q | V | YRPKFQV | 17R |
| 100 | Y | S | P | K | F | Q | V | YSPKFQV | 20EL |
| 100 | Y | D | P | K | F | R | V | YDPKFRV | 6EV |
| 100 | Y | D | P | K | F | Q | A | YDPKFQA | 6EI, 17L |
| 100 | Y | D | P | K | F | Q | D | YDPKFQD | 17L |
| 100 | Y | D | P | K | F | Q | E | YDPKFQE | 20EL |
| 100 | Y | D | P | K | F | Q | G | YDPKFQG | 17V |
| 100 | Y | D | P | K | F | Q | H | YDPKFQH | 20EL |
| 100 | Y | D | P | K | F | Q | K | YDPKFQK | 17L, 17I |
| 100 | Y | D | P | K | F | Q | L | YDPKFQL | 17V |
| 100 | Y | D | P | K | F | Q | M | YDPKFQM | 20EL |
| 100 | Y | D | P | K | F | Q | N | YDPKFQN | 20EL |
| 100 | Y | D | P | K | F | Q | P | YDPKFQP | 17L, 17I, 17V |
| 100 | Y | D | P | K | F | Q | R | YDPKFQR | 17L, 17I |
| 100 | Y | D | P | K | F | Q | S | YDPKFQS | 17L |
| 100 | Y | D | P | K | F | Q | T | YDPKFQT | 17L, 17I |

TABLE 9
Heavy Chain CDR3 Screening Summary (L8
template; 1-46*02 framework)

| [b-TL1A] | 95 | 96 | 97 | 98 | 99 | 101 | 102 | |
|---|---|---|---|---|---|---|---|---|
| (pM) | S | G | G | L | P | D | V | CDR SEQ |
| 500 | L | G | G | L | P | D | V | LGGLPDV |
| 500 | S | A | G | L | P | D | V | SAGLPDV |
| 500 | S | G | G | A | P | D | V | SGGAPDV |
| 500 | S | G | G | M | P | D | V | SGGMPDV |
| 500 | S | G | G | L | P | E | V | SGGLPEV |
| 500 | S | G | G | L | P | D | K | SGGLPDK |
| 500 | S | G | G | L | P | D | M | SGGLPDM |
| 500 | S | G | G | L | P | D | Q | SGGLPDQ |
| 500 | S | G | G | L | P | D | R | SGGLPDR |
| 500 | S | G | G | L | P | D | S | SGGLPDS |
| 500 | S | G | G | L | P | D | T | SGGLPDT |
| 500 | S | G | G | L | P | D | W | SGGLPDW |

TABLE 10
Light Chain CDR1 Screening Summary (L8 template; paired with
heavy chain 1-46*02 framework)

| [b-TL1A] | 24 | 25 | 26 | 27 | 29 | 30 | 31 | 32 | 33 | 34 | |
|---|---|---|---|---|---|---|---|---|---|---|---|
| (pM) | R | A | S | S | S | V | S | Y | M | Y | CDR SEQ |
| 150 | G | A | S | S | S | V | S | Y | M | Y | GASSSVSYMY |
| 150 | W | A | S | S | S | V | S | Y | M | Y | WASSSVSYMY |
| 150 | R | A | S | S | S | V | I | Y | M | Y | RASSSVIYMY |
| 150 | R | A | S | S | S | V | S | F | M | Y | RASSSVSFMY |
| 150 | R | A | S | S | S | V | S | Y | L | Y | RASSSVSYLY |
| 150 | R | A | S | S | S | V | S | Y | M | R | RASSSVSYMR |

TABLE 11
Light Chain CDR2 Screening Summary (L8 template;
paired with heavy chain 1-46*02 framework)

| [b-TL1A] | 50 | 51 | 52 | 53 | 54 | 55 | 56 | |
|---|---|---|---|---|---|---|---|---|
| (pM) | A | T | S | N | L | A | S | CDR SEQ |
| 150 | A | K | S | N | L | A | S | AKSNLAS |
| 150 | A | T | P | N | L | A | S | ATPNLAS |
| 150 | A | T | E | N | L | A | S | ATENLAS |
| 150 | A | T | S | L | L | A | S | ATSLLAS |
| 150 | A | T | S | P | L | A | S | ATSPLAS |
| 150 | A | T | S | N | L | T | S | ATSNLTS |

TABLE 12
Light Chain CDR3 Screening Summary (various templates; paired
with heavy chain 1-46*02 framework)

| [b-TL1A] | 89 | 90 | 91 | 92 | 93 | 94 | 95 | 96 | 97 | | |
|---|---|---|---|---|---|---|---|---|---|---|---|
| (pM) | Q | Q | W | S | G | N | P | R | T | CDR SEQ | Template |
| 200 | H | Q | W | S | G | N | P | R | T | HQWSGNPRT | L8 |
| 200 | N | Q | W | S | G | N | P | R | T | NQWSGNPRT | L8 |
| 200 | S | Q | W | S | G | N | P | R | T | SQWSGNPRT | L8 |
| 200 | Q | Q | S | S | G | N | P | R | T | QQSSGNPRT | L8 |
| 200 | Q | Q | W | D | G | N | P | R | T | QQWDGNPRT | L8 |
| 200 | Q | Q | W | E | G | N | P | R | T | QQWEGNPRT | L8 |
| 200 | Q | Q | W | H | G | N | P | R | T | QQWHGNPRT | L8 |
| 200 | Q | Q | W | N | G | N | P | R | T | QQWNGNPRT | L8 |
| 200 | Q | Q | W | Q | G | N | P | R | T | QQWQGNPRT | L8 |
| 200 | Q | Q | W | V | G | N | P | R | T | QQWVGNPRT | L8 |
| 200 | Q | Q | W | S | A | N | P | R | T | QQWSANPRT | L8 |
| 200 | Q | Q | W | S | D | N | P | R | T | QQWSDNPRT | L8 |
| 200 | Q | Q | W | S | Q | N | P | R | T | QQWSQNPRT | L8 |
| 200 | Q | Q | W | S | S | N | P | R | T | QQWSSNPRT | L8 |
| 500 | Q | Q | W | S | G | N | P | R | S | QQWSGNPRS | L8 |
| 500 | Q | Q | F | S | G | N | P | R | T | QQFSGNPRT | 16 |
| 500 | Q | Q | H | S | G | N | P | R | T | QQHSGNPRT | 46 |
| 500 | Q | Q | I | S | G | N | P | R | T | QQISGNPRT | 16 |
| 500 | Q | Q | P | S | G | N | P | R | T | QQPSGNPRT | 16 |
| 500 | Q | Q | R | S | G | N | P | R | T | QQRSGNPRT | 46 |
| 500 | Q | Q | Y | S | G | N | P | R | T | QQYSGNPRT | 46 |
| 500 | Q | Q | W | S | G | H | P | R | T | QQWSGHPRT | 16, 46 |
| 500 | Q | Q | W | S | G | L | P | R | T | QQWSGLPRT | 46 |
| 500 | Q | Q | W | S | G | Q | P | R | T | QQWSGQPRT | 46 |
| 500 | Q | Q | W | S | G | S | P | R | T | QQWSGSPRT | 16, 46 |
| 500 | Q | Q | W | S | G | T | P | R | T | QQWSGTPRT | 46 |
| 500 | Q | Q | W | S | G | M | P | R | T | QQWSGMPRT | 16, 46 |
| 500 | Q | Q | W | S | G | F | P | R | T | QQWSGFPRT | 46 |
| 500 | Q | Q | W | S | G | K | P | R | T | QQWSGKPRT | 46 |
| 500 | Q | Q | W | S | G | R | P | R | T | QQWSGRPRT | 46 |
| 1000 | Q | Q | W | S | G | D | P | R | T | QQWSGDPRT | L8 |
| 1000 | Q | Q | W | S | G | T | P | R | T | QQWSGTPRT | L8 |

TABLE 13
Heavy Chain CDR3 Screening Summary (L8mod
template; heavy chain 1-3*01 related framework)

| [b-TL1A] | 95 | 96 | 97 | 98 | 99 | 101 | 102 | |
|---|---|---|---|---|---|---|---|---|
| (pM) | S | G | G | L | P | D | V | CDR SEQ |
| 15 | S | G | G | L | P | D | H | SGGLPDH |
| 15 | S | G | G | L | P | D | R | SGGLPDR |
| 15 | S | G | G | L | P | D | F | SGGLPDF |
| 15 | S | G | G | L | P | D | V | SGGLPDV |
| 15 | S | G | G | S | P | D | V | SGGSPDV |

TABLE 14
Light Chain CDR3 Screening Summary (L8mod template; paired
with heavy chain 1-3*01 related framework)

| [b-TL1A] | 89 | 90 | 91 | 92 | 93 | 94 | 95 | 96 | 97 | |
|---|---|---|---|---|---|---|---|---|---|---|
| (pM) | Q | Q | W | S | G | N | P | R | T | CDR SEQ |
| 15 | Q | Q | W | V | G | N | P | R | T | QQWVGNPRT |
| 15 | Q | Q | W | A | G | N | P | R | T | QQWAGNPRT |
| 15 | Q | Q | W | Y | G | N | P | R | T | QQWYGNPRT |
| 15 | Q | Q | W | S | G | N | P | R | T | QQWSGNPRT |
| 15 | Q | Q | W | F | G | N | P | R | T | QQWFGNPRT |
| 15 | Q | Q | W | Q | G | N | P | R | T | QQWQGNPRT |
| 15 | Q | Q | W | S | Q | N | P | R | T | QQWSQNPRT |

### Example 3. Generation and Characterization of Anti-TL1A Antibodies Having Optimized CDRs Using a Mutated (S93) Heavy Chain

Clones containing the CDR-grafted heavy chain (CDR-grafted and 21-3) had lower binding activity than clones that contained the murine back mutation(S) at heavy chain position 93. For this reason, an additional HCDR3 library was constructed. The library was constructed as described above except the degenerate oligonucleotides for all positions were mixed prior to mutagenesis and the library was synthesized and expressed as a pool, as opposed to examining each position separately. Similar to the HCDR3 positional scanning performed on the L8 heavy chain backbone (S93A), position 102 of HCDR3 yielded multiple mutations that enhanced antigen binding in the capture lift format. Screening the HCDR3 library based on heavy chain S93 template identified some mutations that were identified on the heavy chain A93 template, but also identified novel mutations not previously identified (heavy chain CDR3 V1021 and V102Y, for example). A summary of the capture lift screening and DNA sequencing is shown in Table 15.

TABLE 15
Capture lift screening
of HCDR3 library pool on heavy chain template with S93.

| Mutation | Frequency |
|---|---|
| V102K | 10 |
| V102M | 7 |
| V102Y | 4 |
| V102L | 2 |
| V102I | 1 |
| V102E | 1 |
| V102T | 1 |

### Example 4. Identification and Engineering of Potential Sequence Liabilities

A structural homology model of the variable region of CDR-grafted construct L8, based on known PDB antibody structures, was built using Molecular Operating Environment (MOE) 2018.01 software (Chemical Computing Group, Montreal, Canada). The model and BioMOE prediction algorithms were used to perform a sequence liability assessment. In addition, variants 12835 and L8 were analyzed for potential sequence liabilities based on known potentially labile sequence motifs (Jarasch et al., J. Pharm. Sci. 104:1885-1898 (2015); Sydow et al., PLOS ONE 9: e100736 (2014); Vlasak and Ionescu mAbs 3:253-263 (2011)). Using these approaches, multiple residues were identified as potentially labile, including: light chain M33. W35, W47, W91 and N94; heavy chain D31T32, M34, D52P52a, M69 and W103 (summarized in Table 17; FR designates framework).

Exhaustive capture lift screening at all potentially labile sites located within CDRs was performed to identify amino acid substitutions that can eliminate these residues while preserving antigen binding. As an example, one screen was focused on light chain CDR3 N94 (potential deamidation site). The initial capture lift screen of LCDR3 positions 94 did not identify mutations that displayed enhanced affinity relative to the wild-type sequence. Therefore, in order to identify acceptable mutations to eliminate these potentially labile residues the conditions of the initial capture lift screen were altered. Specifically, instead of screening with antigen at a concentration where the wild-type sequence did not provide a signal the antigen concentration was raised so that clones expressing the wild-type sequence were visible on the lift. In this way, variants that bind with affinities similar to the wild-type sequence, but eliminate the problematic residue, can be identified.

For the LCDR3 position 94 library, approximately 3,000 clones were plated and assayed by capture lift. The capture lift was screened using 1000 pM human TL1A and plaques displaying the eight darkest and six lighter staining intensities were picked and sequenced. The results are summarized in Table 16. As expected, two of the darkest staining plaques expressed the wild-type residue, N94. However, the other six dark staining plaques expressed T94, indicating that the variant N94T largely preserves the binding affinity of the wild-type sequence while eliminating the potential deamidation site. In addition, five different sequences were identified from the lighter staining plaques. These included D, F, K, R and S. Although these alternative sequences may be somewhat lower in affinity than the wild-type sequence, all may also serve as replacements for N94 when combined with other, higher affinity mutations identified elsewhere.

TABLE 16
Identification of Alternative Residues at Potentially
Labile Site LCDR3 N94 (screened with 1000 pM antigen)

| | Capture | Nucleo- | | Amino | |
|---|---|---|---|---|---|
| | Lift | tide | | acid | |
| Clone | Staining | Sequence | DNA | Sequence | Protein |
| L8 | Dark | AAT | — | N | — |
| L3-6-01, -04 | Dark | ACT | SEQ ID 75 | T | SEQ ID 76 |
| L3-6-02, -07 | Dark | AAT | — | N | — |
| L3-6-03, -05, | Dark | ACG | SEQ ID 77 | T | SEQ ID 76 |
| -06, -08 | | | | | |
| L3-6-09 | Light | GAT | SEQ ID 79 | D | SEQ ID 78 |
| L3-6-10 | Light | TTT | SEQ ID 81 | F | SEQ ID 80 |
| L3-6-11, -12 | Light | AAG | SEQ ID 83 | K | SEQ ID 82 |
| L3-6-14 | Light | CGG | SEQ ID 85 | R | SEQ ID 84 |
| L3-6-15 | Light | TCT | SEQ ID 87 | S | SEQ ID 86 |

TABLE 17
Summary of potentially labile residues and active
variants that eliminate the lability

| Chain | Residue (Location) | Alternative(s) | Comments |
|---|---|---|---|
| Light | M33 (CDR1) | L | |
| | W35 (FR2) | | |
| | W47 (FR2) | I | Changed to I to |
| | | | match human |
| | | | germline sequence |
| | W91 (CDR3) | | |
| | N94 (CDR3) | D, F, K, R, S, T; | |
| | | H, L, M, Q | |
| Heavy | D31T32 (CDR1) | A32 | |
| | M34 (CDR1) | I, L | |
| | D52P52a (CDR2) | E52 | |
| | M69 (FR3) | 169 | Removed in all variants |
| | | | based on alternative |
| | | | heavy chain framework |
| | | | VH1-3*01 |
| | W103 (FR4) | | |

### Example 5. Identification of Mutations that Confer Enhanced Expression in E. coli

Certain mutations identified during the screening of CDR libraries by capture lift did not always demonstrate enhanced binding in the ELISA format, but consistently expressed soluble Fab in the periplasmic space of bacteria at higher levels than other variants. In particular, this phenomenon was observed at heavy chain CDR2 position V65 (V65G, V65T, and V65K) and light chain CDR1 position R24 (R24G). These results were surprising because the capture lift screening format is configured to minimize the impact of different expression levels, while maximizing the impact of affinity on the signal intensity. Consequently, these mutations were noted and integrated into later combinatorial libraries that included mutations that enhanced affinity in order to determine if the mutations would confer expression and/or thermostability benefits to candidates expressed as intact immunoglobulins in mammalian expression systems.

### Example 6. Generation and Characterization of Anti-TL1A Antibodies Having Combinatorial HCDR3 and LCDR3 Mutations

Based on the initial identification of beneficial mutations in both HCDR3 and LCDR3 an additional library was synthesized, expressed, and screened to identify combinations of independent mutations that could further improve the binding affinity. The library was constructed by two site mutagenesis using oligonucleotides encoding a subset of the mutations identified in the positional scanning. Oligonucleotides encoding the wild-type residue were also included. This combinatorial library contained 30 distinct variants: wild-type (no mutations), 9 variants containing a single mutation (redundant with variants identified in positional screen, as shown in Tables 4 and 5), and 20 unique combinations. Capture lift screening with 200 pM antigen identified 21 active clones. DNA sequencing of the 21 clones identified certain combinations more frequently than others (Table 18).

TABLE 18
Combinatorial library screening and DNA sequence summary

| | | HCDR3 V102X | | | | |
|---|---|---|---|---|---|---|
| | | M | K | Q | W | V |
| LCDR3 | D | 4 | 4 | 0 | 0 | 0 |
| S92X | E | 0 | 1 | 0 | 1 | 0 |
| | H | 0 | 0 | 0 | 0 | 0 |
| | N | 0 | 4 | 0 | 1 | 0 |
| | Q | 1 | 0 | 1 | 0 | 0 |
| | S | 0 | 3 | 0 | 1 | 0 |

Subsequently, multiple combinatorial libraries were synthesized, expressed and screened (details below). In general, these libraries combined mutations that were identified as improving affinity (Examples 2 and 3) with mutations that altered potentially labile residues (Example 4) and with mutations that potentially conferred enhanced thermostability/expression (Example 5). The combinatorial libraries were screened in multiple ELISA formats to identify clones with the best attributes for further development (affinity, selectivity, binding to membrane-associated TL1A, and developability). Multiple variants with optimized and diverse CDR sequences utilizing different VH germline templates were identified as summarized in Tables 19 to 22.

TABLE 19
Heavy Chain CDRs on 1-46*02 Heavy chain template

| Clone | SEQ | HCDR1 | SEQ | HCDR2 (50-65), | SEQ | HCDR3 |
|---|---|---|---|---|---|---|
| ID | ID | (26-35) | ID | (1^{st} P is 52a | ID | (93-102) |
| Start | 553 | GFDIQDTYMH | 554 | RIDPASGHTKYDPKFQV | 565 | ARSGGLPDV |
| 34 | 553 | GFDIQDTYMH | 555 | RIEPASGHIKYDPKFQG | 566 | ARSGGLPDW |
| 2 | 553 | GFDIQDTYMH | 556 | RIEPASGHIKYSPKFQG | 566 | ARSGGLPDW |
| 52 | 553 | GFDIQDTYMH | 556 | RIEPASGHIKYSPKFQG | 566 | ARSGGLPDW |
| 46 | 553 | GFDIQDTYMH | 557 | RIEPASGHVKYSPKFQV | 566 | ARSGGLPDW |
| 47 | 553 | GFDIQDTYMH | 558 | RIEPASGHVKYDPKFQT | 566 | ARSGGLPDW |
| 14 | 553 | GFDIQDTYMH | 559 | RIDPASGHIKYDPKFQK | 567 | ARSGGLPDM |
| 16 | 553 | GFDIQDTYMH | 560 | RIDPASGHVKIDPKFQV | 567 | ARSGGLPDM |
| 17L | 553 | GFDIQDTYMH | 561 | RIDPASGHLKYDPKFQV | 567 | ARSGGLPDM |
| 17L-1 | 553 | GFDIQDTYMH | 562 | RIDPASGHLKYDPKFQR | 567 | ARSGGLPDM |
| 23 | 553 | GFDIQDTYMH | 563 | RIDPASGHLKYDPKFQN | 568 | ARSGGLPDK |
| A1 | 553 | GFDIQDTYMH | 563 | RIDPASGHLKYDPKFQN | 568 | ARSGGLPDK |
| 53 | 553 | GFDIQDTYMH | 564 | RIEPASGHLKYDPKFQE | 568 | ARSGGLPDK |
| E1 | 553 | GFDIQDTYMH | 564 | RIEPASGHLKYDPKFQE | 568 | ARSGGLPDK |
| | 484 | DTYMH | 485 | PASGH | 486 | SGGLPD |

TABLE 20
Light Chain CDRs on 3-20*01 Light chain template

| Clone | SEQ | LCDR1 | SEQ | LCDR2 | SEQ | LCDR3 |
|---|---|---|---|---|---|---|
| ID | ID | (24-33) | ID | (50-56) | ID | (89-97) |
| Start | 569 | RASSSVSYMY | 488 | ATSNLAS | 571 | QQWSGNPRT |
| 34 | 569 | RASSSVSYMY | 488 | ATSNLAS | 572 | QQWEGNPRT |
| 2 | 569 | RASSSVSYMY | 488 | ATSNLAS | 572 | QQWEGNPRT |
| 52 | 570 | GASSSVSYMY | 488 | ATSNLAS | 572 | QQWEGNPRT |
| 46 | 569 | RASSSVSYMY | 488 | ATSNLAS | 572 | QQWEGNPRT |
| 47 | 569 | RASSSVSYMY | 488 | ATSNLAS | 572 | QQWEGNPRT |
| 14 | 569 | RASSSVSYMY | 488 | ATSNLAS | 573 | QQWQGNPRT |
| 16 | 569 | RASSSVSYMY | 488 | ATSNLAS | 573 | QQWQGNPRT |
| 17L | 569 | RASSSVSYMY | 488 | ATSNLAS | 573 | QQWQGNPRT |
| 17L-1 | 569 | RASSSVSYMY | 488 | ATSNLAS | 573 | QQWQGNPRT |
| 23 | 569 | RASSSVSYMY | 488 | ATSNLAS | 572 | QQWEGNPRT |
| A1 | 570 | GASSSVSYMY | 488 | ATSNLAS | 572 | QQWEGNPRT |
| 53 | 569 | RASSSVSYMY | 488 | ATSNLAS | 572 | QQWEGNPRT |
| E1 | 570 | GASSSVSYMY | 488 | ATSNLAS | 572 | QQWEGNPRT |

| | | |
|---|---|---|
| SEQ ID | SEQ ID | SEQ ID |
| NO: 487 | NO: 488 | NO: 489 |
| ASSSVSYMY | ATSNLAS | GNPRT |

TABLE 21
Heavy Chain CDRs on 1-3*01 Heavy chain template

| Clone | SEQ | HCDR1 | SEQ | HCDR2 (50-65), | SEQ | HCDR3 |
|---|---|---|---|---|---|---|
| ID | ID | (26-35) | ID | (1^{st} P is 52a | ID | (93-102) |
| Start | 553 | GFDIQDTYMH | 554 | RIDPASGHTKYDPKFQV | 578 | ARSGGLPDV |
| 3-17L | 553 | GFDIQDTYMH | 574 | RIDPASGHLKYDPKFQG | 579 | ARSGGLPDM |
| V-A | | | | | | |
| 3-17L | 553 | GFDIQDTYMH | 574 | RIDPASGHLKYDPKFQG | 579 | ARSGGLPDM |
| L8mod | 553 | GFDIQDTYMH | 575 | RIDPASGHTKYDPKFQG | 578 | ARSGGLPDV |
| X-V | 553 | GFDIQDTYMH | 554 | RIDPASGHTKYDPKFQV | 580 | ARSGGLPDF |
| X | 553 | GFDIQDTYMH | 554 | RIDPASGHTKYDPKFQV | 580 | ARSGGLPDF |
| H3-1 | 553 | GFDIQDTYMH | 575 | RIDPASGHTKYDPKFQG | 581 | ARSGGLPDL |
| XL3-6 | 553 | GFDIQDTYMH | 575 | RIDPASGHTKYDPKFQG | 580 | ARSGGLPDF |
| XL3- | 553 | GFDIQDTYMH | 575 | RIDPASGHTKYDPKFQG | 580 | ARSGGLPDF |
| 10 | | | | | | |
| XL3- | 553 | GFDIQDTYMH | 575 | RIDPASGHTKYDPKFQG | 580 | ARSGGLPDF |
| 15 | | | | | | |
| L3-13 | 553 | GFDIQDTYMH | 575 | RIDPASGHTKYDPKFQG | 580 | ARSGGLPDF |
| H2-2 | 553 | GFDIQDTYMH | 576 | RIDPASGHSKYDPKFQV | 580 | ARSGGLPDF |
| H2-5 | 553 | GFDIQDTYMH | 577 | RIDPASGHYKYDPKFQV | 580 | ARSGGLPDF |

TABLE 22
Light Chain CDRs on 3-20*01 Light chain template

| Clone | SEQ | LCDR1 | SEQ | LCDR2 | SEQ | LCDR3 |
|---|---|---|---|---|---|---|
| ID | ID | (24-33) | ID | (50-56) | ID | (89-97) |
| Start | 569 | RASSSVSYMY | 488 | ATSNLAS | 571 | QQWSGNPRT |
| 3-17L | 569 | RASSSVSYMY | 488 | ATSNLAS | 573 | QQWQGNPRT |
| V-A | | | | | | |
| 3-17L | 569 | RASSSVSYMY | 488 | ATSNLAS | 573 | QQWQGNPRT |
| L8mod | 569 | RASSSVSYMY | 488 | ATSNLAS | 571 | QQWSGNPRT |
| X-V | 569 | RASSSVSYMY | 488 | ATSNLAS | 571 | QQWSGNPRT |
| X | 569 | RASSSVSYMY | 488 | ATSNLAS | 571 | QQWSGNPRT |
| H3-1 | 569 | RASSSVSYMY | 488 | ATSNLAS | 571 | QQWSGNPRT |
| XL3-6 | 569 | RASSSVSYMY | 488 | ATSNLAS | 582 | SQWSGNPRT |
| XL3-10 | 569 | RASSSVSYMY | 488 | ATSNLAS | 583 | QQWSGNPRS |
| XL3-15 | 569 | RASSSVSYMY | 488 | ATSNLAS | 584 | QQWSRNPRT |
| L3-13 | 569 | RASSSVSYMY | 488 | ATSNLAS | 585 | QQWKGNPRT |
| H2-2 | 569 | RASSSVSYMY | 488 | ATSNLAS | 571 | QQWSGNPRT |
| H2-5 | 569 | RASSSVSYMY | 488 | ATSNLAS | 571 | QQWSGNPRT |

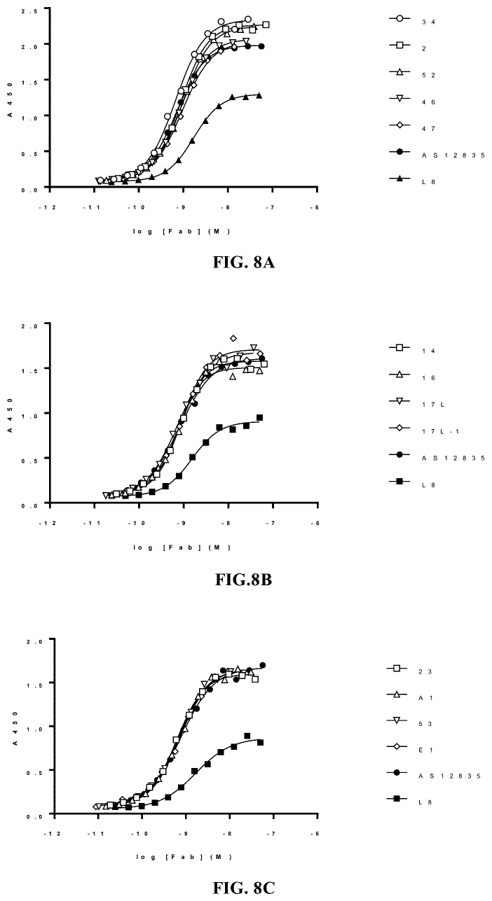

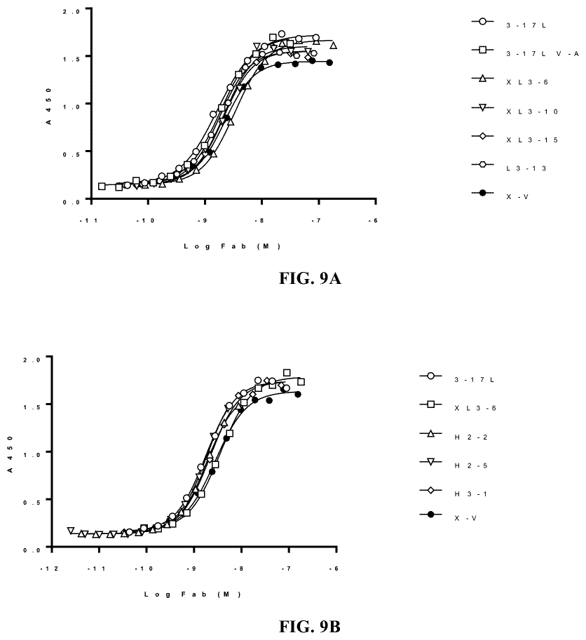

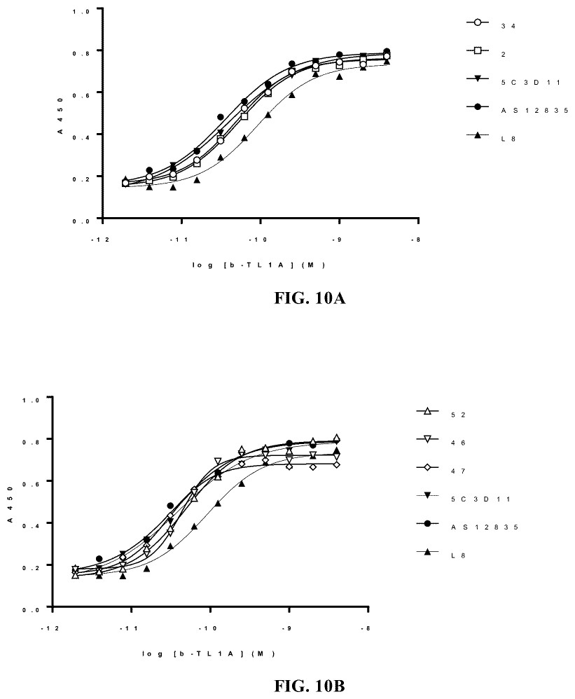

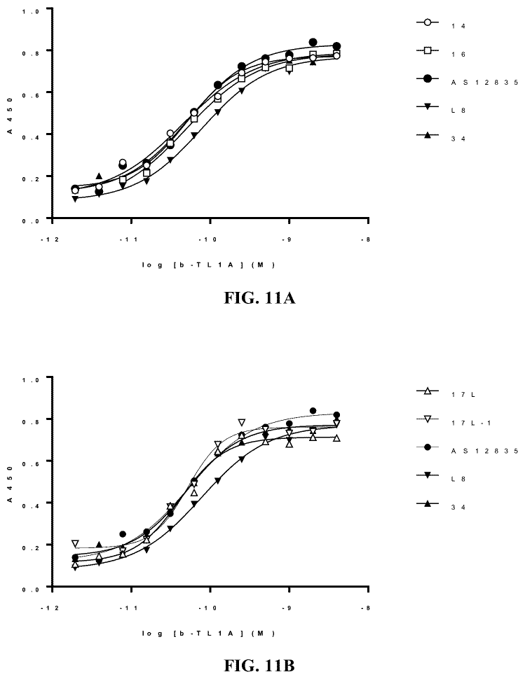

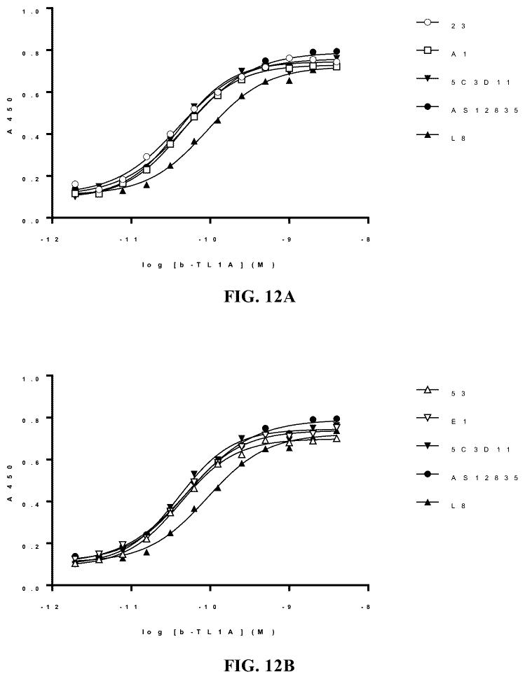

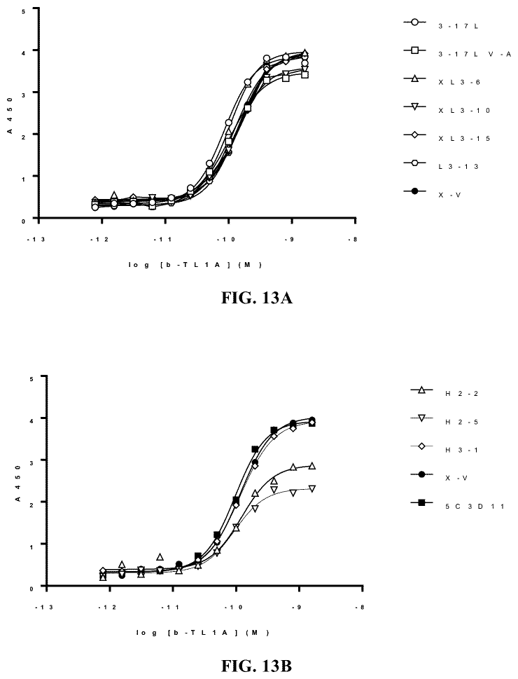

Fabs with the CDRs shown in Tables 19 to 22 were tested for binding to human TL1A in multiple formats. First, human TL1A was immobilized on the surface of an ELISA plate and soluble Fab variants were titrated, as shown in FIGS. 8 and 9. Next, uniform (saturating) quantities of soluble Fab variants were captured on the surface of an ELISA plate and soluble, biotinylated human TL1A was titrated as shown in FIGS. 10 to 13. In both ELISA formats all the Fab variants bound human TL1A and displayed significantly enhanced binding relative to the CDR-grafted variant, L8. In addition, the variants all bound as well, or better than, variant 12835 while having no, or significantly fewer, murine back-mutations in the frameworks. As a result these experiments elucidated a set of anti-TL1A variable regions that exhibited both high binding affinity and high homology to human germline Ig sequences.

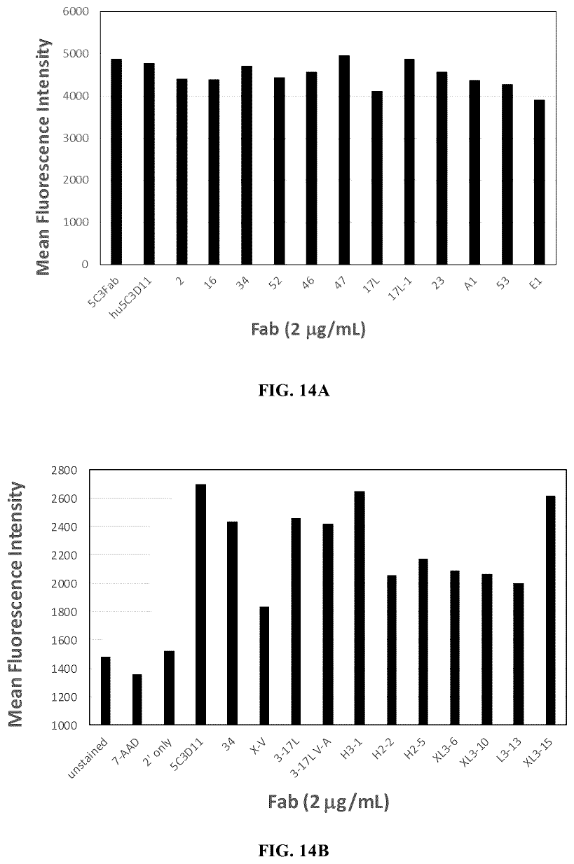

All the Fab variants were tested for binding to membrane-associated human TL1A. For these studies a HEK293 cell line that had been transfected with human TL1A was used. The 293 cells expressing membrane bound human TL1A were maintained in DMEM containing L-glutamine, glucose, sodium pyruvate, and phenol red (ThermoFisher cat #11995-065) plus 10% fetal bovine serum, 1X penicillin-streptomycin (Fisher cat #15140122) and 2 μg/ml puromycin (Gibco cat #A11138-03) in a 37° C. incubator with 5% CO2. Three days prior to the assay, a T-75 flask was seeded with 3×10⁶ cells so that the flask was 90-95% confluent on the day of the assay. The media was aspirated and the cell monolayer was gently washed with 5 ml PBS. Adherent cells were removed by repeatedly pipetting 10 ml ice cold 1% BSA/PBS against the monolayer. The cells were counted and 5×10⁵ were aliquoted for each sample to be analyzed. The cells were collected by centrifuging at 300× g at 4° C. for 5 minutes and the wash was discarded. The cells were resuspended in 100 μl Fab or IgG diluted in 1% BSA/PBS and placed on ice for 30 min. Next, the cells were washed with 1 ml 1% BSA/PBS and collected by centrifuging at 300× g at 4° C. for 5 min. The wash was discarded and 100 μl secondary goat F (ab′) 2 anti-human kappa FITC (Southern Biotech cat #2062-02) or goat F (ab′) 2 anti-human IgG PE (Southern Biotech cat #2043-09) conjugate, diluted 1:200 in BSA/PBS was added. Cells were placed on ice for 30 min. Finally, the cells were washed with 1 ml BSA/PBS, collected by centrifuging at 300× g at 4° C. for 5 min. The wash was removed and the cells were resuspended in 500 μl 1% BSA/PBS. One drop of Sytox AADvanced ReadyFlow Reagent (ThermoFisher cat #R37173) was added per sample and the samples were analyzed on the Attune NXT Flow Cytometer (ThermoFisher). All of the variants bound membrane-associated human TL1A, as shown in FIG. 14.

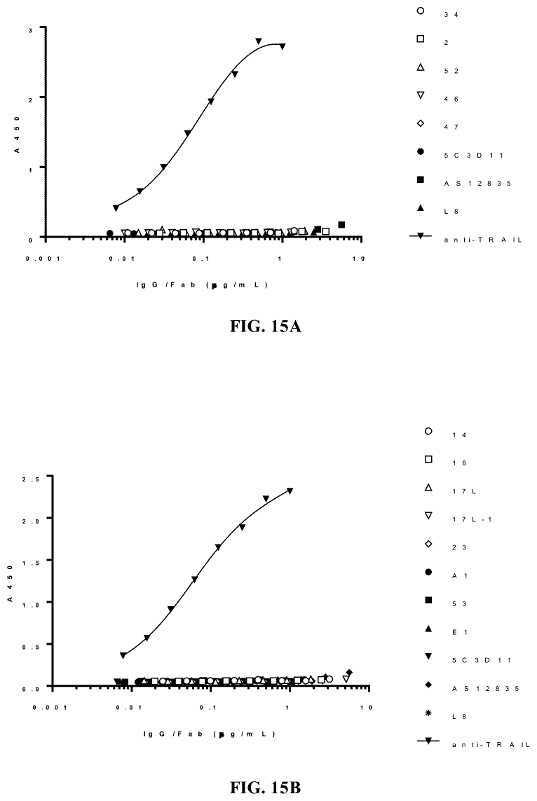

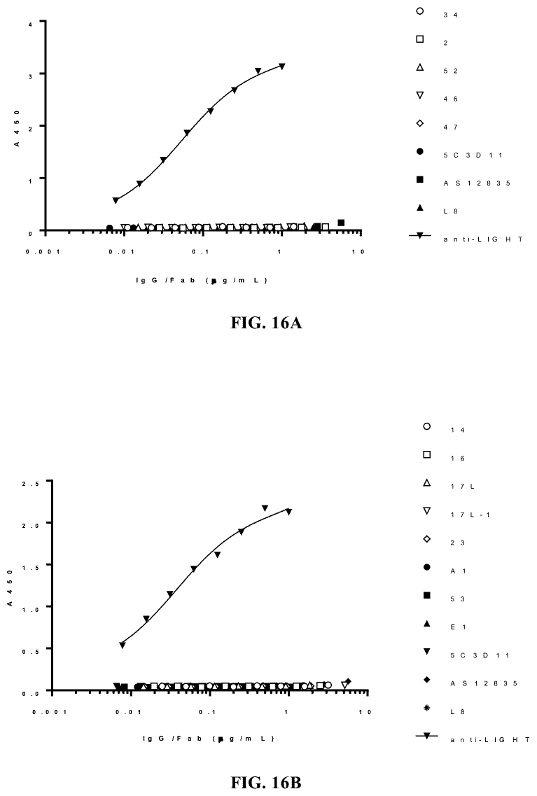

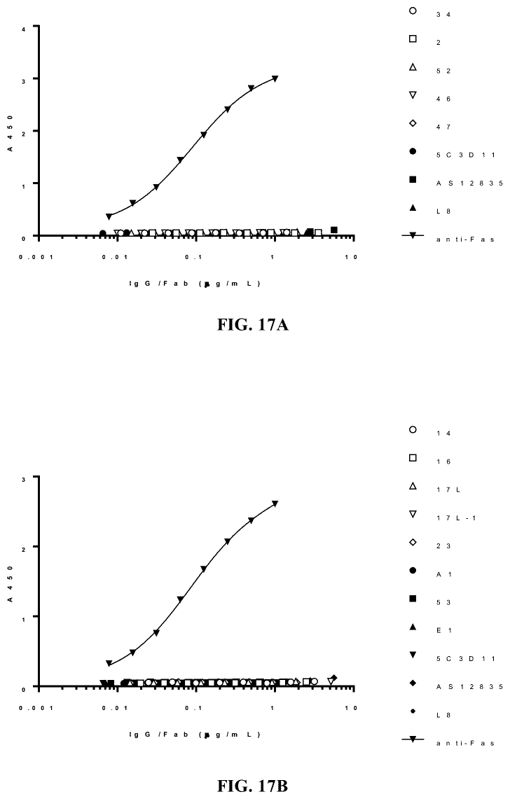

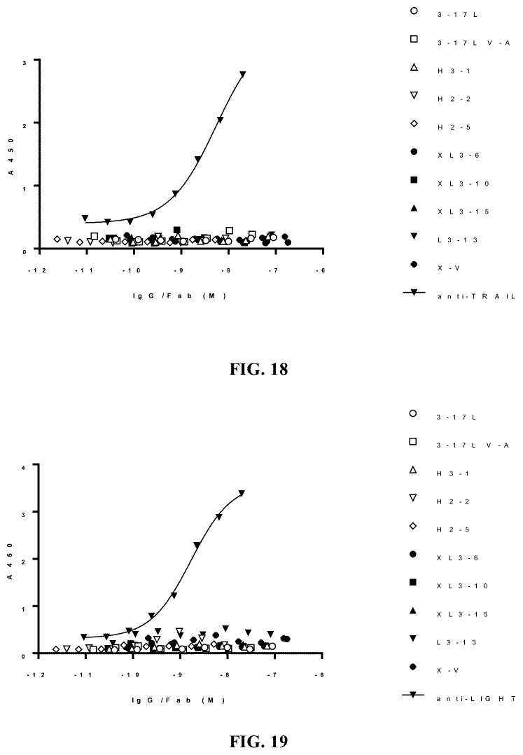

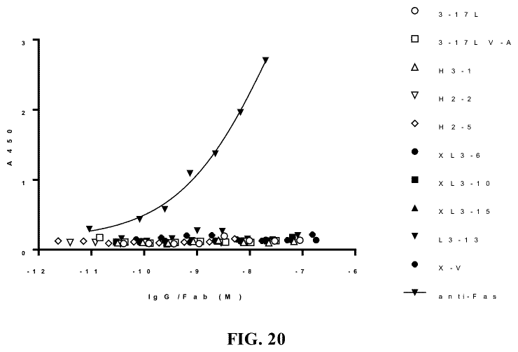

Next, all the Fab variants were characterized for their selectivity for human TL1A relative to other TNFSF members TRAIL, LIGHT and Fas. Briefly, ELISA plates were coated overnight at 4° C. with 50 μl/well antigen (Fas/TNFSF6, R&D Systems, cat. no. 126-FL/CF; TRAIL/TNFSF10, R&D Systems, cat. no. 375-TL/CF; LIGHT/TNFSF14, R&D Systems, cat. no. 664-LI/CF) at 1 μg/ml in PBS. The plate was washed 3 times with PBS-T and blocked with 100 μl of 1% BSA/PBS. The block was discarded and the Fab variants or control antibodies (Fas/TNFSF6, R&D Systems, cat. no. AF126; TRAIL/TNFSF10, R&D Systems, cat. no. AF375; LIGHT/TNFSF14, R&D Systems, cat. no. AF664) were titrated in 50 μl 1% BSA/PBS and incubated for 1 h at 25° C. The plate was washed 3 times with PBS-T and secondary HRP-conjugated antibody (diluted 5,000-fold in 1% BSA/PBS) was added for 1 h at 25° C. The plate was washed 3 times with PBS-T and developed. As shown in FIGS. 15 to 20 none of the variants displayed detectable binding to the related family members, indicating the selectivity for human TL1A versus other TNFSF family members was preserved while engineering higher affinity using human germline framework templates.

### Example 7: Characterization of Select Humanized Variants Expressed on Different IgG Constant Regions

The light and heavy chain variable regions of clones 14, 17L, 23, 34, 47, and 53, from Tables 19 and 20 above, were cloned onto kappa light chain constant region, and either a modified IgG1 or an IgG2 heavy chain backbone, respectively. The modified IgG1 backbone and IgG2 were selected to reduce potential effector function of the antibodies. Transient expression and purification characteristics are shown in Table 23 below. For these variants, all expressed better as modified IgG1 than as IgG2. Furthermore, the yields obtained were consistent with the observation made regarding the impact of certain mutations on expression in bacteria (see Example 5). Specifically, the highest expressing variants 14, 34, and 47 all contained mutations at heavy chain CDR2 V65G, V65T or V65K while the lowest expressing variants 17L, 23, and 53 did not.

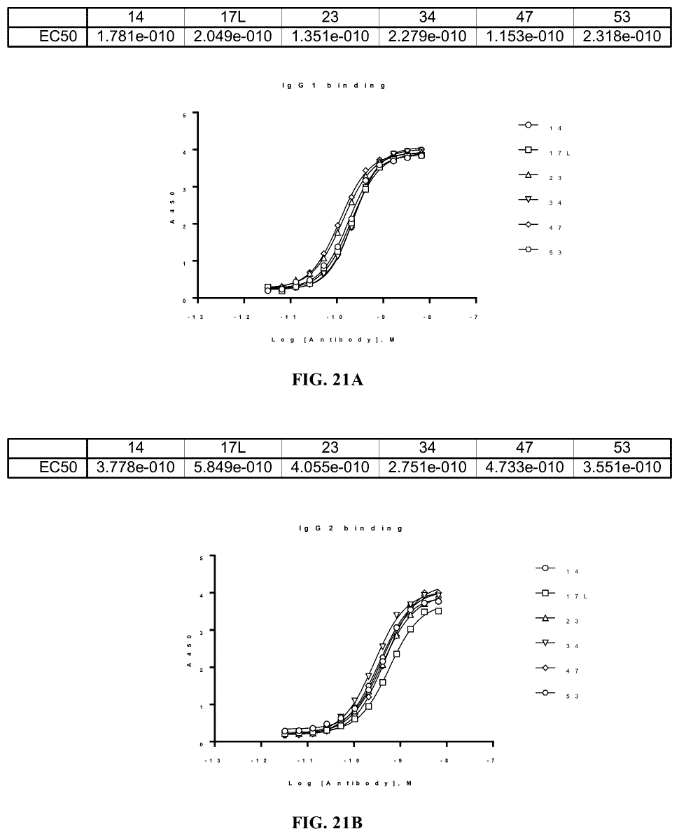

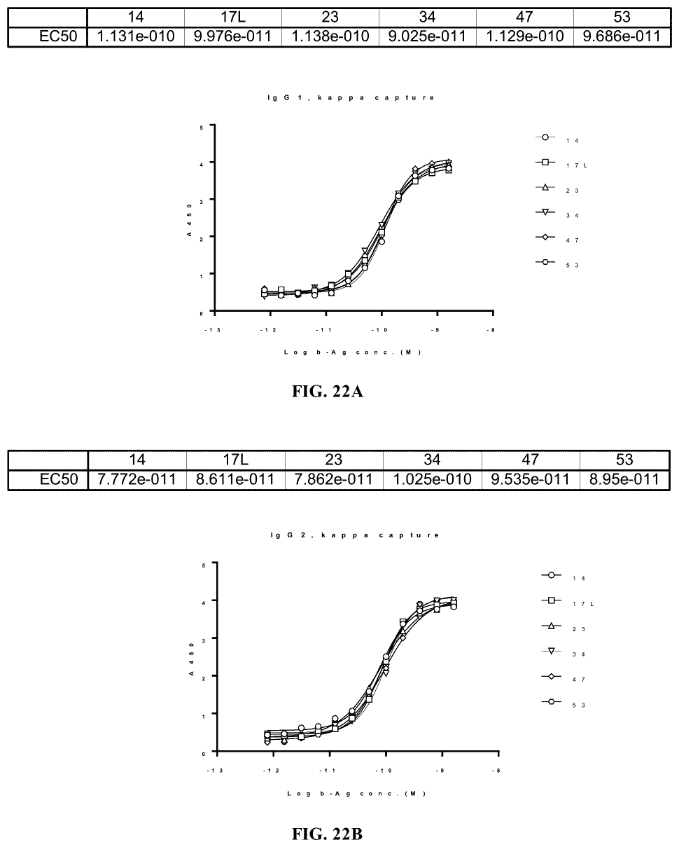

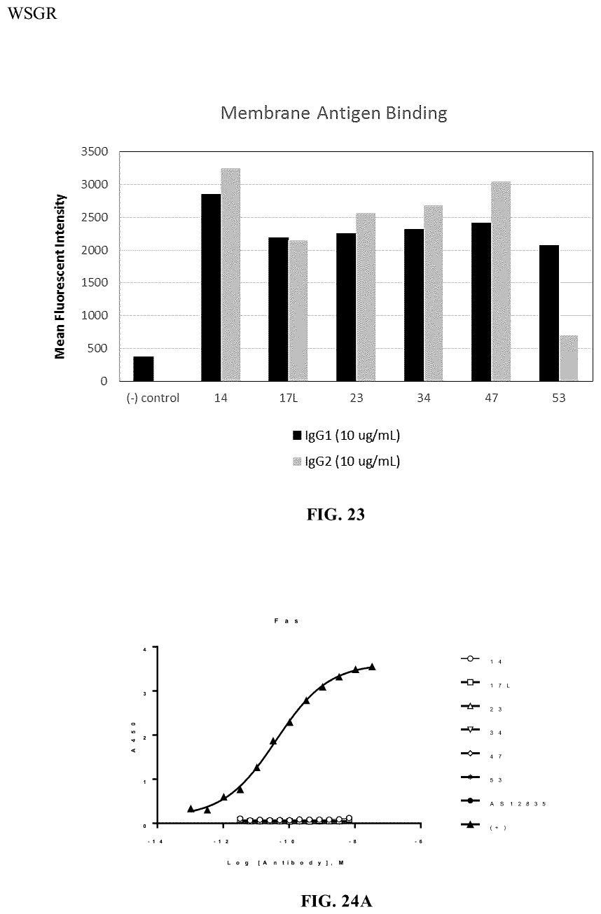

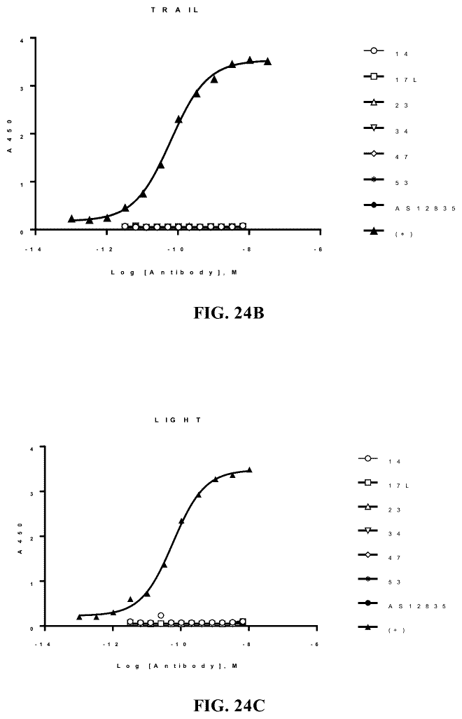

In general, the binding of all variants to human TL1A in both formats (modified IgG1 and IgG2) was preserved, as assessed by ELISA binding to antigen coated plates (FIG. 21) and by ELISA capture of soluble, biotinylated antigen (FIG. 22). In addition, binding was to membrane-associated human TL1A was preserved for all variants, with the exception of variant 53 when expressed as an IgG2 (FIG. 23). Finally, selectivity for human TL1A versus other TNFSF members was maintained, as none of the clones showed appreciable binding to TNFSF family members Fas, TRAIL, or LIGHT (FIG. 24).

TABLE 23
Expression of Select CDR Variants as human IgG1
(modified) and IgG2

| | IgG1 Yield | IgG1 | IgG2 Yield | |
|---|---|---|---|---|
| Clone | (mg) | Purity | (mg) | IgG2 Purity |
| 34 | 5.9 | 90% | 3.1 | 90% |
| 47 | 5.5 | 95% | 3.2 | 95% |
| 14 | 6.1 | 90% | 2.6 | 95% |
| 17L | 0.15 | 90% | 0.03 | <80% |
| 23 | 3.0 | 80% | 0.6 | 80% |
| 53 | 1.8 | 95% | 0.1 | 85% |

### Example 8: Characterization of Potency and Species Selectivity in Whole Blood Assay

The neutralizing activity and potency of the variants described herein expressed as IgG1 (modified) and IgG2 was tested in a human whole blood assay using healthy donors. This assay is a modification of Cassatella et al., “Soluble TNF-like cytokine (TL1A) production by immune complexes stimulated monocytes in rheumatoid arthritis” J Immunol. 2007 Jun. 1; 178 (11): 7325-33; and measures the production of IFN-γ under conditions where TL1A and its receptor DR3 are upregulated and activated. In this assay, both soluble and membrane-associated TL1A are produced. Results from this assay have been shown to correlate with in vivo outcomes in a mouse model of colitis. See Takedatsu, “TL1A (TNFSF15) regulates the development of chronic colitis by modulating both T-helper 1 and T-helper 17 activation. Gastroenterology. 2008 August; 135 (2): 552-567.

Briefly, 96-well plates are coated with human gamma globulin in PBS overnight at 4° C., washed with PBS, and incubated with anti-human IgG (Fc fragment specific) for at least 1 hour at 25° C. to generate immune complex (IC). Just prior to use, the plates are washed three times with PBS. Collected blood samples are treated with IL-12 and IL-18, antibody is titrated in the samples, and the samples are added to the plates and placed at 37° C. for 24 hours. Next, 100 μl of PBS/5% BSA was added to each well and mixed. Plates were centrifuged at 500 g for 5 minutes and then ˜150 μl of diluted plasma was collected for IFN-γ measurement. PBS is added to make collection of a-cellular plasma easier from the plate since the blood percentage is high (95%). All samples are diluted to ensure that values are within the linear range of the standard curve. All variants, regardless of the format of IgG (modified IgG1 or IgG2), displayed potent inhibition of IFN-γ production (Table 24). For comparison, the typical IC50 values for murine parental antibody 5C3D11 and humanized 12835 are 1.38+0.95 nM (donor n=16) and 9.28+10.71 nM (donor n=4), respectively.

TABLE 24
Potency in Human Whole Blood Assay

| | IgG | Mean IC50 +/− SD | |
|---|---|---|---|
| Clone | Subclass | (nM) | # Donors |
| 34 | G1 | 0.18 ± 0.05 | 6 |
| 34 | G2 | 0.16 ± 0.06 | 3 |
| 47 | G1 | 1.26 ± 0.40 | 6 |
| 47 | G2 | 1.01 ± 0.16 | 3 |
| 14 | G1 | 0.19 ± 0.06 | 6 |
| 14 | G2 | 0.35 ± 0.13 | 3 |
| 23 | G1 | 0.41 ± 0.10 | 6 |
| 23 | G2 | 0.33 ± 0.12 | 3 |
| 53 | G1 | 0.39 ± 0.06 | 6 |

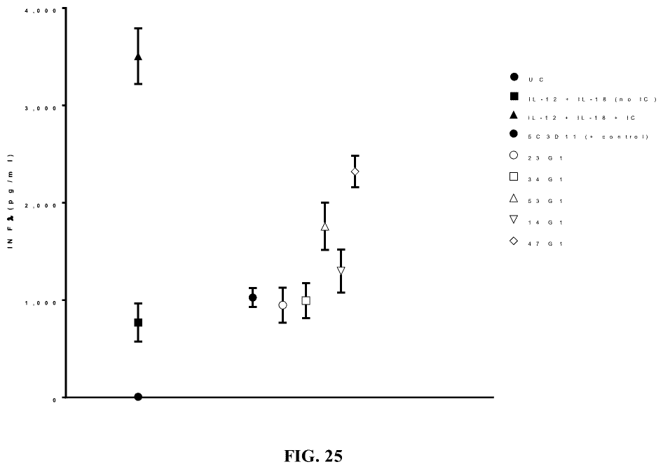

Next, the samples were evaluated in the same assay, using blood obtained from cynomolgus monkeys, in order to evaluate the cross-reactivity of the optimized, humanized variants with cynomolgus TL1A. The assay was performed similar to the assay that utilized human whole blood, except the variants were tested at a single concentration (10 nM), rather than performing a full titration. The variants all inhibited IFN-γ production, though variants 47 and 53 did not inhibit to the extent of variants 14, 23, or 34 and murine 5C3D11 (FIG. 25). These data demonstrate that the optimized humanized variants preserved cross-reactivity with cynomolgus TL1A.

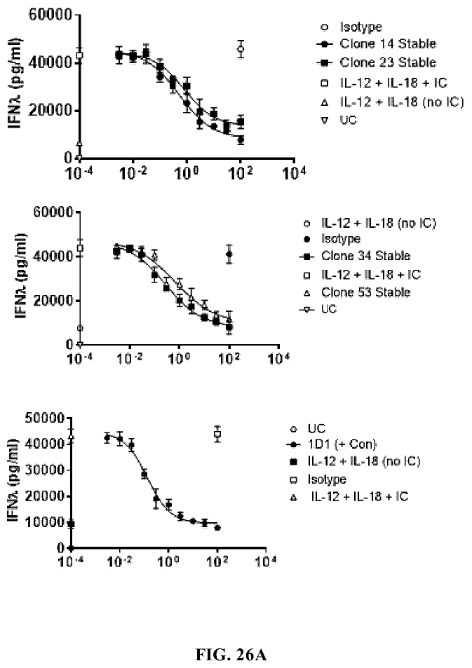

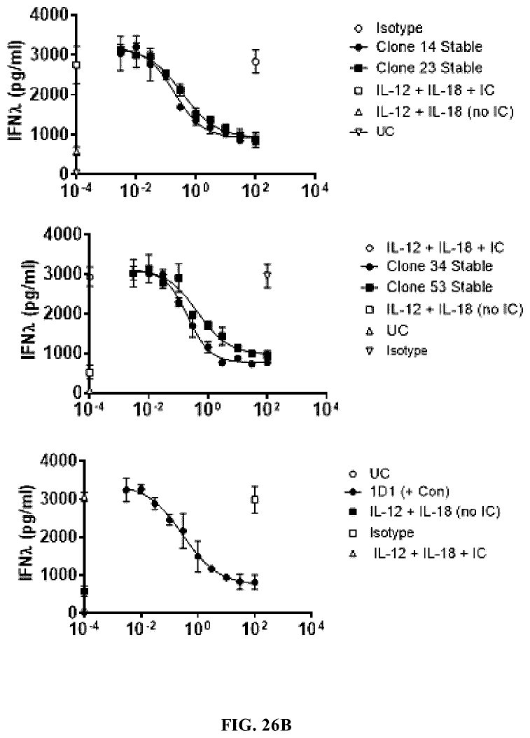

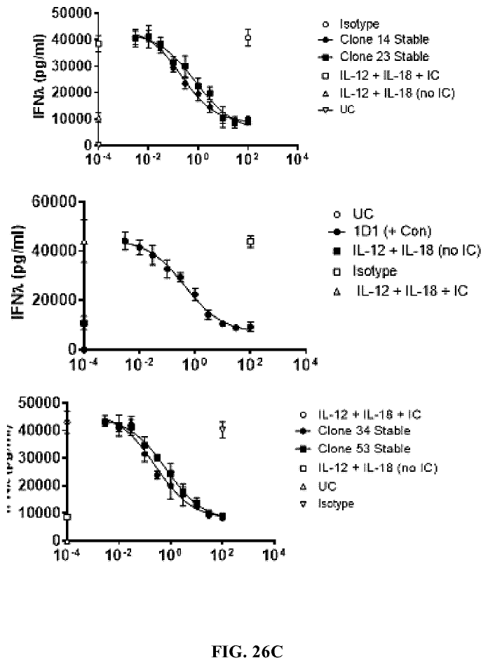

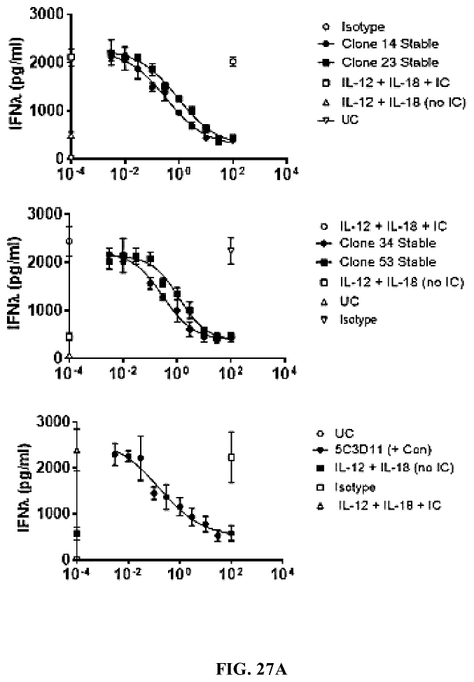

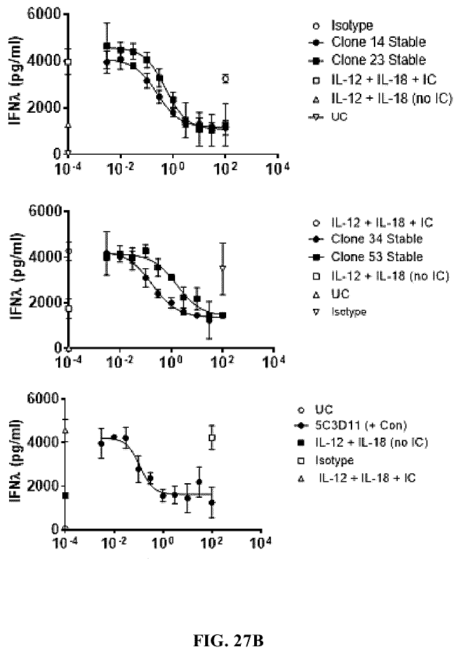

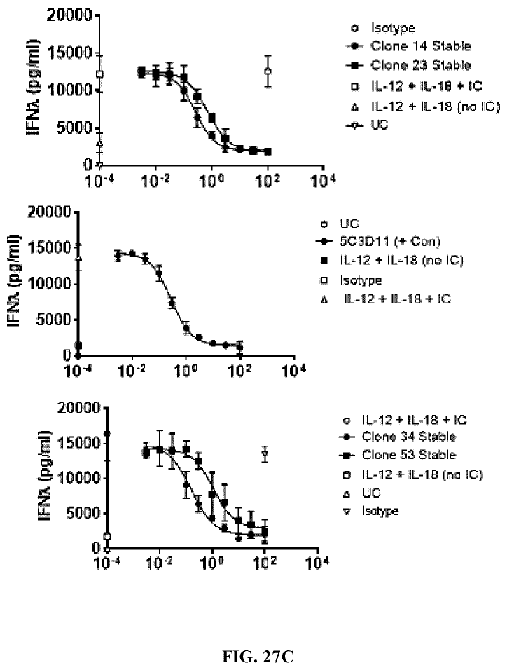

Neutralizing TL1A antibodies were also formatted as effectorless IgG1 (as shown in SEQ ID NO: 542) molecules, expressed and purified from CHO cells, and tested in a potency assay using human (FIG. 26A-C) and cynomolgus monkey (FIG. 27A-C) whole blood as described above. Results are summarized below in Table 25.

TABLE 25
Potency (IC50, nM) of CHO Expressed Variants in Human and
Cynomolgus Whole Blood Assay

| | Human Whole Blood | | | Cynomolgus Whole Blood | | |
|---|---|---|---|---|---|---|
| Clone | Mean | SD | Donors | Mean | SD | Donors |
| 14 | 0.30 | 0.15 | 3 | 0.28 | 0.04 | 3 |
| 23 | 0.64 | 0.21 | 3 | 0.72 | 0.16 | 3 |
| 34 | 0.24 | 0.03 | 3 | 0.21 | 0.06 | 3 |
| 53 | 0.59 | 0.11 | 3 | 1.32 | 0.23 | 3 |
| 1D1 | 0.27 | 0.12 | 3 | | | |
| 5C3D11 | | | | 0.18 | 0.07 | 3 |

These experiments establish that all variants are active and potent, with variants 14 and 34 typically displaying the greatest potency with human blood. All variants are potent using cynomolgus blood. Variants 14, 23 and 34 display similar potency to human and cynomolgus TL1A, while variant 53 displays ˜2-fold greater potency towards human TL1A.

### Example 9: Competition Assays

A binding competition assay using surface plasmon resonance (SPR) is performed to evaluate whether a test anti-TL1A antibody binds to the same region on TL1A as any anti-TL1A antibody described herein.

The reference antibody is directly immobilized via amine coupling onto a carboxymethylated dextran sensor chip surface (CMS) using a Biacore 2000 or 3000 instrument. Recombinant soluble human TL1A or murine TL1A diluted to 10 nM in 8.1 mM Na₂HPO₄, 1.47 mM KF₂PO₄, pH 7.2, 237 mM NaCl, 2.7 mM KCl, 3.4 mM EDTA and 0.01% Tween 20 (PBS-NET) is injected for about 1 minute at a flow rate of 10 RI/minute to achieve binding levels on the immobilized antibody of at least 100 response units (RU). The reference antibody is then injected at 30 nM for 5 minutes in order to saturate all of the potential binding sites on the TL1A. A repeat injection of the reference antibody is performed to confirm this saturation. Next, the test antibody in PBS-NET or PBS-NET alone as a control is injected at 30 nM for 5 minutes. If the test antibody binds to the TL1A saturated with the first antibody, this indicates that the test antibody binds to a non-competing region on TL1A as compared to the reference antibody. If the test antibody does not bind to the saturated TL1A, this indicates that the two antibodies bind to the same region or compete with binding to TL1A. This strategy may be repeated with the test antibody immobilized and the reference antibody injected after the test antibody is bound with TL1A. Each cycle may be repeated. At the end of each cycle, the immobilized antibody surface is regenerated either by a 30-second pulse of 3M MgCl₂ or by 0.1% TFA followed by two consecutive 15-second pulses of PBS-NET. All injections are performed at 25° C. at a collection rate of 10 Hz. All sensorgrams are double referenced by using both a control surface and buffer injections.

Another binding competition assay using SPR is performed to evaluate whether a test anti-TL1A antibody binds to the same region on TL1A as any anti-TL1A antibody described herein. The reference antibody is immobilized to the SPR chip via amine coupled at three or four different densities across the array. The TL1A protein is injected in an increasing concentration series to estimate kinetic parameters and the appropriate concentration for injections during the competition binning experiment. Once the optimal antigen concentration for the binning experiment is determined, regeneration conditions (typically a brief low pH injection) are evaluated to establish the optimal conditions for regeneration between cycles of the binning assay.

Binning is performed using a pre-mix approach, where a moderate concentration of TL1A is injected over the array, either by itself, or pre-complexed to the test antibody at saturating antibody concentrations (e.g., 30-50 μg/mL). The assay may be performed such that the test antibody is immobilized and the reference antibody is pre-complexed to the TL1A. Clones that bind to unique regions from the immobilized antibody provide an increase in signal, while competitive clones will decrease the antigen binding signal. The competition assay is run so that all clones are tested as both ligands and analytes.

### Example 10: Comparison of 5C3D11 Binding to Other Anti-TL1A Antibodies

Two epitope binning studies were performed to compare the epitope recognized by 5C3D11 and 12835 to the epitopes recognized by other TL1A antibodies, including 1D1, 1681, 1B4, and 1A9, as shown in Table 26.

TABLE 26
Antibody variable region sequences used for epitope binning studies

| Antibody | Heavy Chain | Light Chain |
|---|---|---|
| 5C3D11 | SEQ ID NO 3 | SEQ ID NO 6 |
| 12835 | SEQ ID NO 26 | SEQ ID NO 28 |
| 1D1 | QVQLVQSGAEVKKPGASVKVSCKASGYDF | EIVLTQSPATLSLSPGERATLSC |
| | TYYGISWVRQAPGQGLEWMGWISTYNGNT | RASQSVSSYLAWYQQKPGQAPRL |
| | HYARMLQGRVTMTTDTSTRTAYMELRSLR | LIYDASNRATGIPARFSGSGSGT |
| | SDDTAVYYCARENYYGSGAYRGGMDVWGQ | DFTLTISSLEPEDFAVYYCQQRS |
| | GTTVTVSS | NWPWTFGQGTKVEIK |
| 1681 | EVQLLESGGGLVQPGKSLRLSCAVSGFTF | DIQMTQSPSTLSASVGDRVTITC |
| | STYGMNWVRQAPGKGLEWVSSISGTGRTT | RASQTISSWLAWYQQTPEKAPKL |
| | YHADSVQGRFTVSRDNSKNILYLQMNSLR | LIYAASNLQSGVPSRFSGSGSGT |
| | ADDTAVYFCTKERGDYYYGVFDYWGQGTL | EFTLTISSLQPDDFATYYCQQYH |
| | VTVSS | RSWTFGQGTKVEIT |
| 1B4 | QVTLKESGPALVKPTQTLTLTCTFSGFSL | DIQLTQSPSFLSASVGDRVTITC |
| | STSNMGVVWIRQPPGKALEWLAHILWDDR | SASSSVNYMHWYQQKPGKAPKLL |
| | EYSNPALKSRLTISKDTSKNQVVLTMTNM | IYSTSNLASGVPSRFSGSGSGTE |
| | DPVDTATYYCARMSRNYYGSSYVMDYWGQ | FTLTISSLQPEDFATYYCHQWNN |
| | GTLVTVSS | YGTFGQGTKVEIKR |
| 1A9 | QIQLVQSGPELKKPGETVKISCKASGYTF | DVLMTQTPLSLPVSLGDQASISC |
| | TTYGMSWVKQAPGKGLKWMGWMNTYSGVT | RSSQNIVHSDGNTYLEWYLQKPG |
| | TYADDFKGRFAFSLETSASTAYMQIDNLK | QSPKLLIYKVSNRFSGVPDRFSG |
| | NEDTATYFCAREGYVFDDYYATDYWGQGT | SGSGTDFTLKISRVEAEDLGIYY |
| | SVTVSS | CFQGSHVPLTFGAGTKLELK |

To minimize avidity effects a planar carboxymethyldextran surface sensor chip was used (Xantec Prod. #SPMXCMDP) in the first study while a HC30M sensor chip was used (Xantec Prod. #SPMXHC30M) in the second study. The running buffer for continuous flow micro-spotting was HBS-EP+ and the flow rate was 65 μl/min. The chip was activated with 7 min of 18 mM EDC and 4.5 nM sulfo-NHS in 100 mM MES, pH 5.5. Antibodies were then immobilized for 15 min in two replicate prints. Antibodies were diluted to 10 μg/ml in 10 mM acetate, pH 4.5. The antibodies were titrated in a 3-fold serial dilution three places across the plate, establishing a concentration series of different spot densities. Each antibody was spotted 8 times-twice at each of four dilutions. This created a 10×8 array. The remaining active groups were neutralized with a 7 min quench using 1 M ethanolamine, pH 8.5. Because the antigen is a homotrimeric protein and IgG is bivalent the epitope binning was performed using pre-mix conditions.

First epitope binning study-TL1A was prepared at a final concentration of 50 nM (3.3 μg/ml) and mixed with 333 nM (50 μg/ml) analyte (solution phase antibody) or with running buffer (control). For samples in IgG format, 50 μg/ml is 333 nM while for samples in Fab format 50 μg/ml is 1 μM. Mixed samples were injected for 5 min over the array and regenerated for 30 sec after every cycle using a 4:1 mixture of Pierce IgG Elution buffer and 5 M NaCl (1 M final concentration).

Second epitope binning study-TL1A was prepared at a final concentration of 50 nM (3.3 g/ml) and mixed with 1 μM (150 μg/ml) IgG or 2 μM (200 μg/ml) Fab analyte (solution phase antibody) or with running buffer (control). The antibody samples were serially diluted 2-fold seven times (7.8 nM final for IgG, 15 nM final for Fab). Mixed samples were injected for 5 min over the array and regenerated for 30 sec after every cycle using a 4:1 mixture of Pierce IgG Elution buffer and 5 M NaCl (1 M final concentration).

In epitope binning studies a clear signal (sandwich) was observed with immobilized 5C3D11 and 12835 with all of the control antibodies tested as analyte (Table 27, top two rows). These results demonstrate that 5C3D11 and 12835 can bind TL1A simultaneously with the other antibodies and thus, recognize a distinct epitope.

TABLE 27
Summary of ability of antibodies to form sandwich (Yes)

| | | Analyte | | | | | |
|---|---|---|---|---|---|---|---|
| Epitope Bin | Ligand | 5C3D11 | 12835 | 1D1 | 1B4 | 1681 | 1A9 |
| 1 | 5C3D11 | No | No | Yes | Yes | Yes | Yes |
| 1 | 12835 | No | No | Yes | Yes | Yes | Yes |
| 2 | 1D1 | Yes | Yes | No | No | Yes | Yes |
| 2 | 1B4 | Yes | Yes | No | No | Yes | Yes |
| 3 | 1681 | Yes | Yes | Yes | Yes | No | Yes |
| 4 | 1A9 | Yes | Yes | Yes | Yes | Yes | No |

Yes indicates that the antibodies are able to simultaneously bind TL1A target

### Example 11: In Vivo Assessment of Anti-TL1A Efficacy

The efficacy of anti-TL1A antibodies in animal models of colitis is performed. Anti-TL1A antibodies are tested in rodent models of acute colitis induced by intrarectal administration of di- or tri-nitrobenzenesulfonic acid (D/TNBS) or oxazolone, and chronic colitis induced by administration of DSS in drinking water or transfer of CD45RBhi T cells. DNBS and oxazolone induce localized ulceration and inflammation. DSS administration induces robust generalized inflammation of the intestinal tract characterized by erosive lesions and inflammatory infiltrate. Symptoms of all these models usually include diarrhea, occult blood, weight loss and occasionally rectal prolapse. In a prophylactic model, antibody treatment begins at the start of administration of the colitis-inducing compound. In a therapeutic model, antibody treatment begins several days after commencement of induction. The effect of the treatment on weight, stool consistency and occult blood, as well as microscopic effects on epithelial integrity and degree of inflammatory infiltrate is determined. Daily clinical scoring is performed based on stool consistency and presence of occult blood giving a disease activity index (DAI) score.

### Example 12: Phase 1 Clinical Trial

A phase 1 clinical trial is performed to evaluate the safety, tolerability, pharmacokinetics and pharmacodynamics of an anti-TL1A antibody provided herein in subjects having Crohn's Disease.

Single ascending dose (SAD) arms: Subjects in each group (subjects are grouped based on the presence or absence of a risk variant) receive either a single dose of the antibody or a placebo. Exemplary doses are 1, 3, 10, 30, 100, 300, 600 and 800 mg of antibody, or between 5 to 30 milligrams per kilogram. Safety monitoring and PK assessments are performed for a predetermined time. Based on evaluation of the PK data, and if the antibody is deemed to be well tolerated, dose escalation occurs, either within the same groups or a further group of healthy subjects. Dose escalation continues until the maximum dose has been attained unless predefined maximum exposure is reached or intolerable side effects become apparent.

Multiple ascending dose (MAD) arms: Subjects in each group (subjects are grouped based on the presence or absence of a risk variant) receive multiple doses of the antibody or a placebo. The dose levels and dosing intervals are selected as those that are predicted to be safe from the SAD data. Dose levels and dosing frequency are chosen to achieve therapeutic drug levels within the systemic circulation that are maintained at steady state for several days to allow appropriate safety parameters to be monitored. Samples are collected and analyzed to determination PK profiles.

Inclusion Criteria: Healthy subjects of non-childbearing potential between the ages of 18 and 55 years. Healthy is defined as no clinically relevant abnormalities identified by a detailed medical history, full physical examination, including blood pressure and pulse rate measurement, 12 lead ECG and clinical laboratory tests. Female subjects of non-childbearing potential must meet at least one of the following criteria: (1) achieved postmenopausal status, defined as: cessation of regular menses for at least 12 consecutive months with no alternative pathological or physiological cause; and have a serum follicle stimulating hormone (FSH) level within the laboratory's reference range for postmenopausal females; (2) have undergone a documented hysterectomy and/or bilateral oophorectomy; (3) have medically confirmed ovarian failure. All other female subjects (including females with tubal ligations and females that do NOT have a documented hysterectomy, bilateral oophorectomy and/or ovarian failure) will be considered to be of childbearing potential. Body Mass Index (BMI) of 17.5 to 30.5 kg/m2; and a total body weight >50 kg (110 lbs). Evidence of a personally signed and dated informed consent document indicating that the subject (or a legal representative) has been informed of all pertinent aspects of the study.

Two groups of healthy subjects are selected: subjects having a risk variant whose presence is associated with an increase in susceptibility to Crohn's Disease, and subjects lacking the risk variant.

Exclusion Criteria: Evidence or history of clinically significant hematological, renal, endocrine, pulmonary, gastrointestinal, cardiovascular, hepatic, psychiatric, neurologic, or allergic disease (including drug allergies, but excluding untreated, asymptomatic, seasonal allergies at time of dosing). Subjects with a history of or current positive results for any of the following serological tests: Hepatitis B surface antigen (HBsAg), Hepatitis B core antibody (HBcAb), anti-Hepatitis C antibody (HCV Ab) or human immunodeficiency virus (HIV). Subjects with a history of allergic or anaphylactic reaction to a therapeutic drug. Treatment with an investigational drug within 30 days (or as determined by the local requirement, whichever is longer) or 5 half-lives or 180 days for biologics preceding the first dose of study medication. Pregnant females; breastfeeding females; and females of childbearing potential.

Primary Outcome Measures: Incidence of dose limiting or intolerability treatment related adverse events (AEs) [Time Frame: 12 weeks]. Incidence, severity and causal relationship of treatment emergent AEs (TEAEs) and withdrawals due to treatment emergent adverse events [Time Frame: 12 weeks]. Incidence and magnitude of abnormal laboratory findings [Time Frame: 12 weeks]. Abnormal and clinically relevant changes in vital signs, blood pressure (BP) and electrocardiogram (ECG) parameters [Time Frame: 12 wccks].

Secondary Outcome Measures: Single Ascending Dose: Maximum Observed Plasma Concentration (Cₘₐₓ) [Time Frame: 12 weeks]. Single Ascending Dose: Time to Reach Maximum Observed Plasma Concentration (Tₘₐₓ) [Time Frame: 12 weeks]. Single Ascending Dose: Area under the plasma concentration-time profile from time zero to 14 days (AUC14 days) [Time Frame: 12 weeks]. Single Ascending Dose: Area under the plasma concentration-time profile from time zero extrapolated to infinite time (AUC_{inf}) [Time Frame: 12 weeks]. Single Ascending Dose: Area under the plasma concentration-time profile from time zero to the time of last quantifiable concentration (AUCₗₐₛₜ) [Time Frame: 12 weeks]. Single Ascending Dose: Dose normalized maximum plasma concentration (Cₘₐₓ[dn]) [Time Frame: 12 weeks]. Single Ascending Dose: Dose normalized area under the plasma concentration-time profile from time zero extrapolated to infinite time (AUC_{inf}[dn]) [Time Frame: 12 weeks]. Single Ascending Dose: Dose normalized area under the plasma concentration-time profile from time zero to the time of last quantifiable concentration (AUCₗₐₛₜ[dn]) [Time Frame: 12 weeks]. Single Ascending Dose: Plasma Decay Half-Life (t1/2) [Time Frame: 12 weeks]. Plasma decay half-life is the time measured for the plasma concentration to decrease by one half. Single Ascending Dose: Mean residence time (MRT) [Time Frame: 12 weeks]. Single Ascending Dose: Volume of Distribution at Steady State (Vₛₛ) [Time Frame: 6 weeks]. Volume of distribution is defined as theoretical volume in which the total amount of drug would need to be uniformly distributed to produce the desired blood concentration of a drug. Steady state volume of distribution (Vₛₛ) is the apparent volume of distribution at steady-state. Single Ascending Dose: Systemic Clearance (CL) [Time Frame: 6]. CL is a quantitative measure of the rate at which a drug substance is removed from the body.

Multiple Ascending Dose First Dose: Maximum Observed Plasma Concentration (Cₘₐₓ) [Time Frame: 12 weeks]. Multiple Ascending Dose First Dose: Time to Reach Maximum Observed Plasma Concentration (Tₘₐₓ) [Time Frame: 12 weeks]. Multiple Ascending Dose First Dose: Area under the plasma concentration-time profile from time zero to time τ, the dosing interval where τ=2 weeks (AUC_{τ}) [Time Frame: 12 weeks]. Multiple Ascending Dose First Dose: Dose normalized maximum plasma concentration (Cₘₐₓ [dn]) [Time Frame: 12 weeks]. Multiple Ascending Dose First Dose: Dose normalized Area under the plasma concentration-time profile from time zero to time τ, the dosing interval where τ=2 weeks (AUC_{τ}[dn]) [Time Frame: 12 weeks]. Plasma Decay Half-Life (t1/2) [Time Frame: 12 weeks]. Plasma decay half-life is the time measured for the plasma concentration to decrease by one half. Multiple Ascending Dose First Dose: Mean residence time (MRT) [Time Frame: 12 weeks]. Apparent Volume of Distribution (Vz/F) [Time Frame: 12 weeks]. Volume of distribution is defined as theoretical volume in which the total amount of drug would need to be uniformly distributed to produce the desired plasma concentration of a drug. Apparent volume of distribution after oral dose (Vz/F) is influenced by the fraction absorbed. Multiple Ascending Dose First Dose: Volume of Distribution at Steady State (Vₛₛ) [Time Frame: 12 weeks]. Volume of distribution is defined as theoretical volume in which the total amount of drug would need to be uniformly distributed to produce the desired blood concentration of a drug. Steady state volume of distribution (Vₛₛ) is the apparent volume of distribution at steady-state. Multiple Ascending Dose First Dose: Apparent Oral Clearance (CL/F) [Time Frame: 12 weeks]. Clearance of a drug is a measure of the rate at which a drug is metabolized or eliminated by normal biological processes. Clearance obtained after oral dose (apparent oral clearance) is influenced by the fraction of the dose absorbed. Clearance is estimated from population pharmacokinetic (PK) modeling. Drug clearance is a quantitative measure of the rate at which a drug substance is removed from the blood. Multiple Ascending Dose First Dose: Systemic Clearance (CL) [Time Frame: 12 weeks]. CL is a quantitative measure of the rate at which a drug substance is removed from the body.

Multiple Ascending Dose Multiple Dose: Maximum Observed Plasma Concentration (Cₘₐₓ) [Time Frame: 12 weeks]. Multiple Ascending Dose Multiple Dose: Time to Reach Maximum Observed Plasma Concentration (Tₘₐₓ) [Time Frame: 12 weeks]. Multiple Ascending Dose Multiple Dose: Area under the plasma concentration-time profile from time zero to time τ, the dosing interval where τ=2 weeks (AUC_{τ}) [Time Frame: 12 weeks]. Multiple Ascending Dose Multiple Dose: Dose normalized maximum plasma concentration (Cₘₐₓ [dn]) [Time Frame: 12 weeks]. Multiple Ascending Dose Multiple Dose: Dose normalized Area under the plasma concentration-time profile from time zero to time τ, the dosing interval where τ=2 weeks (AUC_{τ}[dn]) [Time Frame: 12 weeks]. Multiple Ascending Dose Multiple Dose: Plasma Decay Half-Life (t1/2) [Time Frame: 12 weeks]. Plasma decay half-life is the time measured for the plasma concentration to decrease by one half. Multiple Ascending Dose Multiple Dose: Apparent Volume of Distribution (Vz/F) [Time Frame: 12 weeks]. Volume of distribution is defined as theoretical volume in which the total amount of drug would need to be uniformly distributed to produce the desired plasma concentration of a drug. Apparent volume of distribution after oral dose (Vz/F) is influenced by the fraction absorbed. Multiple Ascending Dose Multiple Dose: Volume of Distribution at Steady State (Vₛₛ) [Time Frame: 12 weeks]. Volume of distribution is defined as theoretical volume in which the total amount of drug would need to be uniformly distributed to produce the desired blood concentration of a drug. Steady state volume of distribution (Vₛₛ) is the apparent volume of distribution at steady-state.

Multiple Ascending Dose Multiple Dose: Apparent Oral Clearance (CL/F) [Time Frame: 12 weeks]. Clearance of a drug is a measure of the rate at which a drug is metabolized or eliminated by normal biological processes. Clearance obtained after oral dose (apparent oral clearance) is influenced by the fraction of the dose absorbed. Clearance was estimated from population pharmacokinetic (PK) modeling. Drug clearance is a quantitative measure of the rate at which a drug substance is removed from the blood. Multiple Ascending Dose Multiple Dose: Systemic Clearance (CL) [Time Frame: 12 weeks]. CL is a quantitative measure of the rate at which a drug substance is removed from the body. Multiple Ascending Dose Multiple Dose: Minimum Observed Plasma Trough Concentration (Cₘᵢₙ) [Time Frame: 12 weeks]. Multiple Ascending Dose Multiple Dose: Average concentration at steady state (Cav) [Time Frame: 12 weeks]. Multiple Ascending Dose Multiple Dose: Observed accumulation ratio (Rac) [Time Frame: 12 weeks]. Multiple Ascending Dose Multiple Dose: Peak to trough fluctuation (PTF) [Time Frame: 12 weeks]. Multiple Ascending Dose Additional Parameter: estimate of bioavailability (F) for subcutaneous administration at the corresponding intravenous dose [Time Frame: 12 weeks]. Immunogenicity for both Single Ascending Dose and Multiple Ascending Dose: Development of anti-drug antibodies (ADA) [Time Frame: 12 weeks].

### Example 13: Phase 1b Clinical Trial

A phase 1b open label clinical trial is performed to evaluate efficacy of an anti-TL1A antibody provided herein on patients having a risk variant associated with Crohn's Disease.

Arms: 10 patients positive for a risk variant whose presence is associated with an increase in susceptibility to Crohn's Disease are administered the antibody. 5-10 patients negative for the risk variant are administered the antibody. Patients are monitored in real-time. Central ready of endoscopy and biopsy is employed, with readers blinded to point of time of treatment and endpoints.

Inclusion Criteria: Two groups of subjects are selected: subjects having a risk variant whose presence is associated with an increase in susceptibility to Crohn's Disease, and subjects lacking the risk variant.

Primary Outcome Measures: Simple Endoscopic Score for Crohn's Disease (SESCD), Crohn's Disease Activity Index (CDAI), and Patient Reported Outcome (PRO). If risk variant positive group shows 50% reduction from baseline, a Phase 2a clinical trial is performed.

Inclusion Criteria: PRO entry criteria: Abdominal pain score of 2 or more and/or stool frequency score of 4 or more. Primary outcome would be pain core of 0 or 1 and stool frequency score of 3 or less with no worsening from baseline. Endoscopy entry criteria: SESCD ileum only entry at score of 4 and 6 if colon is involved. Primary endoscopic outcome is 40-50% delta of mean SESCD.

### Example 14: Phase 2a Clinical Trial

A phase 2a clinical trial is performed to evaluate the efficacy of an anti-TL1A antibody provided herein in subjects having Crohn's Disease.

Arms: 40 patients per arm (antibody and placebo arms) are treated with antibody or placebo for 12 weeks. An interim analysis is performed after 20 patients from each group are treated at the highest dose to look for a 40-50% delta between placebo and treated group in primary outcome (50% reduction from baseline in SESCD, CDAI, and PRO).

Primary Outcome Measures: Simple Endoscopic Score for Crohn's Disease (SESCD), Crohn's Disease Activity Index (CDAI), and Patient Reported Outcome (PRO).

Inclusion Criteria: PRO entry criteria: Abdominal pain score of 2 or more and/or stool frequency score of 4 or more. Primary outcome would be pain core of 0 or 1 and stool frequency score of 3 or less with no worsening from baseline. Endoscopy entry criteria: SESCD ileum only entry at score of 4 and 6 if colon is involved. Primary endoscopic outcome is 40-50% delta of mean SESCD.

Various embodiments are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments known to the applicant at this time of filing the application has been presented and is intended for the purposes of illustration and description. The present description is not intended to be exhaustive nor limited to the precise form disclosed and many modifications and variations are possible in the light of the above teachings. The embodiments described serve to explain principles and practical applications, and to enable others skilled in the art to utilize the various embodiments, optionally with various modifications, as are suited to the particular use contemplated. Therefore, it is intended that the disclosure not be limited to the particular embodiments disclosed.

While particular embodiments have been shown and described, it will be obvious to those skilled in the art that, based upon the teachings herein, changes and modifications may be made without departing from this disclosure and its broader aspects and, therefore, the appended claims are to encompass within their scope all such changes and modifications as are within the true spirit and scope of this disclosure. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

SEQUENCES

| SEQ | | |
|---|---|---|
| ID | | |
| NO | Description | Sequence |
| 1 | murine mAb 5C3D11 | gaagttcagctgcaacagtctggcgccgagctggttaagcctggcgcttctgtgaagctga |
| | heavy chain variable | gctgtaccgcctctggcttcgacatccaagacacctacatgcactgggtcaagcagaggcc |
| | region | tgagcagggactcgagtggatcggcagaattgatcctgccagcggccacaccaaatacga |
| | | ccccaagttccaagtgaaggccaccatcaccaccgacaccagcagcaataccgcctacct |
| | | gcagctgagcagcctgacctctgaagataccgccgtgtactactgcagcagatctggcgg |
| | | actgcccgatgtttggggagccggaacaaccgtgacagtgtccagc |
| 2 | murine mAb 5C3D11 | gaggttcaacttcaacaatcgggggccgagctggttaagcccggcgcttctgtaaaattgt |
| | heavy chain variable | cttgcactgcctctgggtttgacatccaagatacatatatgcattgggtgaaacagcgtcc |
| | region-codon | cgagcagggcttggagtggattggacgtattgaccccgcctctgggcacacgaaatatgat |
| | optimized for E. | cctaagttccaggttaaagcgactatcacaacggacacctccagcaatacggcttatttac |
| | coli | agttatcctcgctgacctctgaggatactgcagtgtactactgctctcgctctggtggtct |
| | | gccagacgtgtggggtgcaggaactacagttactgtgtcttca |
| 3 | murine mAb 5C3D11 | EVQLQQSGAELVKPGASVKLSCTASGFDIQDTYMHWVK |
| | heavy chain variable | QRPEQGLEWIGRIDPASGHTKYDPKFQVKATITTDTSSNT |
| | region-amino acid | AYLQLSSLTSEDTAVYYCSRSGGLPDVWGAGTTVTVSS |
| 4 | murine mAb 5C3D11 | caaattgtgctgtctcagagccccgccatcctgagtgcttctccaggcgagaaagtgacca |
| | light chain variable | tgacctgcagagccagcagcagcgtgtcctacatgtactggtatcagcagaagcccggcag |
| | region | cagccccaagccttggatctacgccacaagcaatctggccagcggcgtgcccgatagattt |
| | | tctggctctggcagcggcaccagctacagcctgacaatctctagagtggaagccgaggat |
| | | gccgccacctactactgtcaacagtggagcggcaaccccagaacctttggcggaggcac |
| | | caagctggaaatcaag |
| 5 | murine mAb 5C3D11 | caaatcgtcctgtcacagtccccggcgatcctttctgcttcaccaggagagaaggtaacca |
| | light chain variable | tgacatgtcgcgcctatcctcagtttcttacatgtactggtaccagcagaaaccaggatca |
| | region-codon | tctcccaaaccctggatctacgctacatcaaaccttgcatctggcgtgccagaccgttttt |
| | optimized for E. | cagggtcgggctcggggacttcctattcattaaccatttctcgcgtagaagcggaagacgc |
| | coli | cgccacgtattattgtcagcagtggtcaggaaatccgcgcacattcggaggcggaacgaaa |
| | | ttggagatcaaa |
| 6 | murine mAb 5C3D11 | QIVLSQSPAILSASPGEKVTMTCRASSSVSYMYWYQQKPG |
| | light chain variable | SSPKPWIYATSNLASGVPDRFSGSGSGTSYSLTISRVEAED |
| | region-amino acid | AATYYCQQWSGNPRTFGGGTKLEIK |
| 7 | 5C3D11 HCDR1 | ggcttcgacatccaagacacctacatgcac |
| 8 | 5C3D11 HCDR1- | gggtttgacatccaagatacatatatgcat |
| | codon optimized for | |
| | E. coli | |
| 9 | 5C3D11 HCDR1- | GFDIQDTYMH |
| | amino acid | |
| 10 | 5C3D11 HCDR2 | agaattgatcctgccagcggccacaccaaatacgaccccaagttccaagtg |
| 11 | 5C3D11 HCDR2- | cgtattgaccccgcctctgggcacacgaaatatgatcctaagttccaggtt |
| | codon optimized for | |
| | E. coli | |
| 12 | 5C3D11 HCDR2- | RIDPASGHTKYDPKFQV |
| | amino acid | |
| 13 | 5C3D11 HCDR3 | tctggcggactgcccgatgtt |
| 14 | 5C3D11 HCDR3- | tctggtggtctgccagacgtg |
| | codon optimized for | |
| | E. coli | |
| 15 | 5C3D11 HCDR3- | SGGLPDV |
| | amino acid | |
| 16 | 5C3D11 LCDR1 | agagccagcagcagcgtgtcctacatgtac |
| 17 | 5C3D11 LCDR1- | cgcgcctcttcctcagtttcttacatgtac |
| | codon optimized for | |
| | E. coli | |
| 18 | 5C3D11 LCDR1- | RASSSVSYMY |
| | amino acid | |
| 19 | 5C3D11 LCDR2 | gccacaagcaatctggccagc |
| 20 | 5C3D11 LCDR2- | gctacatcaaaccttgcatct |
| | codon optimized for | |
| | E. coli | |
| 21 | 5C3D11 LCDR2- | ATSNLAS |
| | amino acid | |
| 22 | 5C3D11 LCDR3 | caacagtggagcggcaaccccagaacc |
| 23 | 5C3D11 LCDR3- | cagcagtggtcaggaaatccgcgcaca |
| | codon optimized for | |
| | E. coli | |
| 24 | 5C3D11 LCDR3- | QQWSGNPRT |
| | amino acid | |
| 25 | 12835 (humanized | caagtacaattagtccagtcgggtgccgaggtaaaaaaacctggagcatccgtaaaactgt |
| | 5C3D11 heavy chain | cttgcaaagcatcggggtttgacatccaggacacctacatgcactgggtgcgtcaagctcc |
| | variable region)- | aggacagggattagagtggatgggtcgcatcgaccccgcgagcggacacacgaaatac |
| | codon optimized for | gaccctaaatttcaagtacgtgtcacgatgactaccgacactagtacgagcactgtttata |
| | E. coli | tggaattgtcctcgttacgctcagaggatacggcagtctattattgcagccgttccggagg |
| | | cttacccgacgtctggggacagggaactactgtaacagtcagtagt |
| 26 | 12835 (humanized | QVQLVQSGAEVKKPGASVKLSCKASGFDIQDTYMHWVR |
| | 5C3D11 heavy chain | QAPGQGLEWMGRIDPASGHTKYDPKFQVRVTMTTDTSTS |
| | variable region)- | TVYMELSSLRSEDTAVYYCSRSGGLPDVWGQGTTVTVSS |
| | amino acid | |
| 27 | 12835 (humanized | gagattgtgttaacgcaatcaccggggactttatcgctgtcgccgggggagcgcgttacaa |
| | 5C3D11 light chain | tgtcttgtcgcgcttcctcttcggtttcatacatgtattggtatcaacaaaaaccgggac |
| | variable region)- | ggctccacgcccctggatttacgctactagcaatttggcctcgggcgttcccgaccgcttc |
| | codon optimized for | agcgggtcagggagcggcaccgattacacgttgaccatctctcgtctggaacctgaagact |
| | E. coli | tcgcggtctattactgtcaacaatggtcgggaaatccccgtacatttggcggagggacgaa |
| | | gttggaaattaaa |
| 28 | 12835 (humanized | EIVLTQSPGTLSLSPGERVTMSCRASSSVSYMYWYQQKPG |
| | 5C3D11 light chain | QAPRPWIYATSNLASGVPDRFSGSGSGTDYTLTISRLEPED |
| | variable region)- | FAVYYCQQWSGNPRTFGGGTKLEIK |
| | amino acid | |
| 29 | 12835 HCDR1- | gggtttgacatccaggacacctacatgcac |
| | codon optimized for | |
| | E. coli | |
| 30 | 12835 HCDR2- | cgcatcgaccccgcgagcggacacacgaaatacgaccctaaatttcaagta |
| | codon optimized for | |
| | E. coli | |
| 31 | 12835 HCDR3- | tccggaggcttacccgacgtc |
| | codon optimized for | |
| | E. coli | |
| 32 | 12835 LCDR1- | cgcgcttcctcttcggtttcatacatgtattggtat |
| | codon optimized for | |
| | E. coli | |
| 33 | 12835 LCDR2- | gctactagcaatttggcctcg |
| | codon optimized for | |
| | E. coli | |
| 34 | 12835 LCDR3- | caacaatggtcgggaaatccccgtaca |
| | codon optimized for | |
| | E. coli | |
| 35 | 18-7 (CDR-grafted | caagtacaattagtccagtcgggtgccgaggtaaaaaaacctggagcatccgtaaaactgt |
| | light chain) heavy | cttgcaaagcatcggggtttgacatccaggacacctacatgcactgggtgcgtcaagctcc |
| | chain variable | aggacagggattagagtggatgggtcgcatcgaccccgcgagcggacacacgaaatac |
| | region | gaccctaaatttcaagtacgtgtcacgatgactcgtgacactagtacgagcactgtttata |
| | | tggaattgtcctcgttacgctcagaggatacggcagtctattattgcagccgttccggagg |
| | | cttacccgacgtctggggacagggaactactgtaacagtcagtagt |
| 36 | 18-7 (CDR-grafted | QVQLVQSGAEVKKPGASVKLSCKASGFDIQDTYMHWVR |
| | light chain) heavy | QAPGQGLEWMGRIDPASGHTKYDPKFQVRVTMTRDTST |
| | chain variable | STVYMELSSLRSEDTAVYYCSRSGGLPDVWGQGTTVTVS |
| | region-amino acid | S |
| 37 | 18-7 (CDR-grafted | gagattgtgttaacgcaatcaccggggactttatcgctgtcgccgggggagcgcgcgaca |
| | light chain) light | ctgtcttgtcgcgcttcctcttcggtttcatacatgtattggtatcaacaaaaaccggga |
| | chain variable | caggctccacgcctgctgatttacgctactagcaatttggcctcgggcatccccgaccgc |
| | region | ttcagcgggtcagggagcggcaccgattttacgttgaccatctctcgtctggaacctgaa |
| | | gacttcgcggtctattactgtcaacaatggtcgggaaatccccgtacatttggcggaggg |
| | | acgaagttggaaattaaa |
| 38 | 18-7 (CDR-grafted | EIVLTQSPGTLSLSPGERATLSCRASSSVSYMYWYQQKPG |
| | light chain) light | QAPRLLIYATSNLASGIPDRFSGSGSGTDFTLTISRLEPEDF |
| | chain variable | AVYYCQQWSGNPRTFGGGTKLEIK |
| | region-amino acid | |
| 39 | 21-3 (CDR-grafted | caagtacaattagtccagtcgggtgccgaggtaaaaaaacctggagcatccgtaaaagtct |
| | heavy chain) heavy | cttgcaaagcatcggggtttgacatccaggacacctacatgcactgggtgcgtcaagctcc |
| | chain variable | aggacagggattagagtggatgggtcgcatcgaccccgcgagcggacacacgaaatac |
| | region | gaccctaaatttcaagtacgtgtcacgatgactcgtgacactagtacgagcactgtttata |
| | | tggaattgtcctcgttacgctcagaggatacggcagtctattattgcgcacgttccggagg |
| | | cttacccgacgtctggggacagggaactactgtaacagtcagtagt |
| 40 | 21-3 (CDR-grafted | QVQLVQSGAEVKKPGASVKVSCKASGFDIQDTYMHWVR |
| | heavy chain) heavy | QAPGQGLEWMGRIDPASGHTKYDPKFQVRVTMTRDTST |
| | chain variable | STVYMELSSLRSEDTAVYYCARSGGLPDVWGQGTTVTVS |
| | region-amino acid | S |
| 41 | 21-3 (CDR-grafted | gagattgtgttaacgcaatcaccggggactttatcgctgtcgccgggggagcgcgcgaca |
| | heavy chain) light | ctgtcttgtcgcgcttcctcttcggtttcatacatgtattggtatcaacaaaaaccggga |
| | chain variable | caggctccacgcctgctgatttacgctactagcaatttggcctcgggcgttcccgaccgc |
| | region | ttcagcgggtcagggagcggcaccgattacacgttgaccatctctcgtctggaacctgaa |
| | | gacttcgcggtctattactgtcaacaatggtcgggaaatccccgtacatttggcggaggg |
| | | acgaagttggaaattaaa |
| 42 | 21-3 (CDR-grafted | EIVLTQSPGTLSLSPGERATLSCRASSSVSYMYWYQQKPG |
| | heavy chain) light | QAPRLLIYATSNLASGVPDRFSGSGSGTDYTLTISRLEPED |
| | chain variable | FAVYYCQQWSGNPRTFGGGTKLEIK |
| | region-amino acid | |
| 43 | 21-3 V102K (CDR- | QVQLVQSGAEVKKPGASVKVSCKASGFDIQDTYMHWVR |
| | grafted heavy chain) | QAPGQGLEWMGRIDPASGHTKYDPKFQVRVTMTRDTST |
| | heavy chain variable | STVYMELSSLRSEDTAVYYCARSGGLPDKWGQGTTVTVS |
| | region-amino acid | S |
| 44 | 21-3 V102M (CDR- | QVQLVQSGAEVKKPGASVKVSCKASGFDIQDTYMHWVR |
| | grafted heavy chain) | QAPGQGLEWMGRIDPASGHTKYDPKFQVRVTMTRDTST |
| | heavy chain variable | STVYMELSSLRSEDTAVYYCARSGGLPDMWGQGTTVTV |
| | region-amino acid | SS |
| 45 | 21-3 V102Q (CDR- | QVQLVQSGAEVKKPGASVKVSCKASGFDIQDTYMHWVR |
| | grafted heavy chain) | QAPGQGLEWMGRIDPASGHTKYDPKFQVRVTMTRDTST |
| | heavy chain variable | STVYMELSSLRSEDTAVYYCARSGGLPDQWGQGTTVTVS |
| | region-amino acid | S |
| 46 | 21-3 V102W (CDR- | QVQLVQSGAEVKKPGASVKVSCKASGFDIQDTYMHWVR |
| | grafted heavy chain) | QAPGQGLEWMGRIDPASGHTKYDPKFQVRVTMTRDTST |
| | heavy chain variable | STVYMELSSLRSEDTAVYYCARSGGLPDWWGQGTTVTV |
| | region-amino acid | SS |
| 47 | 18-7 S92D (CDR- | EIVLTQSPGTLSLSPGERATLSCRASSSVSYMYWYQQKPG |
| | grafted light chain) | QAPRLLIYATSNLASGIPDRFSGSGSGTDFTLTISRLEPEDF |
| | light chain variable | AVYYCQQWDGNPRTFGGGTKLEIK |
| | region-amino acid | |
| 48 | 18-7 S92E (CDR- | EIVLTQSPGTLSLSPGERATLSCRASSSVSYMYWYQQKPG |
| | grafted light chain) | QAPRLLIYATSNLASGIPDRFSGSGSGTDFTLTISRLEPEDF |
| | light chain variable | AVYYCQQWEGNPRTFGGGTKLEIK |
| | region-amino acid | |
| 49 | 18-7 S92H (CDR- | EIVLTQSPGTLSLSPGERATLSCRASSSVSYMYWYQQKPG |
| | grafted light chain) | QAPRLLIYATSNLASGIPDRFSGSGSGTDFTLTISRLEPEDF |
| | light chain variable | AVYYCQQWHGNPRTFGGGTKLEIK |
| | region-amino acid | |
| 50 | 18-7 S92N (CDR- | EIVLTQSPGTLSLSPGERATLSCRASSSVSYMYWYQQKPG |
| | grafted light chain) | QAPRLLIYATSNLASGIPDRFSGSGSGTDFTLTISRLEPEDF |
| | light chain variable | AVYYCQQWNGNPRTFGGGTKLEIK |
| | region-amino acid | |
| 51 | 18-7 S92Q (CDR- | EIVLTQSPGTLSLSPGERATLSCRASSSVSYMYWYQQKPG |
| | grafted light chain) | QAPRLLIYATSNLASGIPDRFSGSGSGTDFTLTISRLEPEDF |
| | light chain variable | AVYYCQQWQGNPRTFGGGTKLEIK |
| | region-amino acid | |
| 52 | 21-3 CDRv (CDR- | QVQLVQSGAEVKKPGASVKVSCKASGFX₁X₂X₃DTX₄X₅H |
| | grafted heavy chain) | WVRQAPGQGLEWMGRIDPASGHTKYDPKFQVRVTMTR |
| | heavy chain variable | DTSTSTVYMELSSLRSEDTAVYYCARSGGX₆PDX₇WGQGT |
| | region-amino acid | TVTVSS |
| | | X₁ = D or E |
| | | X₂ = I, P, or V |
| | | X₃ = G, Q, S, or V |
| | | X₄ = F or Y |
| | | X₅ = I or M |
| | | X₆ = L or M |
| | | X₇ = E, I, K, L, M, Q, T, V, W, or Y |
| 53 | 18-7 CDRv (CDR- | EIVLTQSPGTLSLSPGERATLSCRASSSVSYMYWYQQKPG |
| | grafted light chain) | QAPRLLIYATSNLASGIPDRFSGSGSGTDFTLTISRLEPEDF |
| | light chain variable | AVYYCX₁QWX₂X₃X₄PRTFGGGTKLEIK |
| | region-amino acid | X₁ = Q or N |
| | | X₂ = D, E, H, N, Q, or S |
| | | X₃ = A or G |
| | | X₄ = D, F, K, N, R, S, or T |
| 54 | 21-3 CDRv (heavy | QVQLVQSGAEVKKPGASVKVSCKASGFX₁X₂X₃DTX₄X₅H |
| | chain contains | WVRQAPGQGLEWMGRIDPASGHTKYDPKFQVRVTMTR |
| | murine S93) heavy | DTSTSTVYMELSSLRSEDTAVYYCSRSGGX₆PDX₇WGQGT |
| | chain variable | TVTVSS |
| | region-amino acid | X₁ = D or E |
| | | X₂ = I, P, or V |
| | | X₃ = G, Q, S, or V |
| | | X₄ = F or Y |
| | | X₅ = I or M |
| | | X₆ = L or M |
| | | X₇ = E, I, K, L, M, Q, T, V, W, or Y |
| 76 | QQWSGTPRT | |
| 78 | QQWSGDPRT | |
| 80 | QQWSGFPRT | |
| 82 | QQWSGKPRT | |
| 84 | QQWSGRPRT | |
| 86 | QQWSGSPRT | |

(L8; VL)
SEQ ID NO: 490
EIVLTQSPGTLSLSPGERATLSCRASSSVSYMYWYQQKPGQAPRLLIYATSNLASGIPDRFS
GSGSGTDFTLTISRLEPEDFAVYYCQQWSGNPRTFGGGTKLEIK
(L8; VH)
SEQ ID NO: 491
QVQLVQSGAEVKKPGASVKVSCKASGFDIQDTYMHWVRQAPGQGLEWMGRIDPASGHTKYDP
KFQVRVTMTRDTSISTVYMELSSLRSEDTAVYYCARSGGLPDVWGQGTTVTVSS
(Clone 34; VL)
SEQ ID NO: 492
EIVLTQSPGTLSLSPGERATLSCRASSSVSYMYWYQQKPGQAPRLLIYATSNLASGIPDRFS
GSGSGTDFTLTISRLEPEDFAVYYCQQWEGNPRTFGGGTKLEIK
(Clone 34; VH)
SEQ ID NO: 493
QVQLVQSGAEVKKPGASVKVSCKASGFDIQDTYMHWVRQAPGQGLEWMGRIEPASGHIKYDP
KFQGRVTMTRDTSISTVYMELSSLRSEDTAVYYCARSGGLPDWWGQGTTVTVSS
(Clone 2; VL)
SEQ ID NO: 494
EIVLTQSPGTLSLSPGERATLSCRASSSVSYMYWYQQKPGQAPRLLIYATSNLASGIPDRFS
GSGSGTDFTLTISRLEPEDFAVYYCQQWEGNPRTFGGGTKLEIK
(Clone 2; VH)
SEQ ID NO: 495
QVQLVQSGAEVKKPGASVKVSCKASGFDIQDTYMHWVRQAPGQGLEWMGRIEPASGHIKYSP
KFQGRVTMTRDTSISTVYMELSSLRSEDTAVYYCARSGGLPDWWGQGTTVTVSS
(Clone 52; VL)
SEQ ID NO: 496
EIVLTQSPGTLSLSPGERATLSCGASSSVSYMYWYQQKPGQAPRLLIYATSNLASGIPDRFS
GSGSGTDFTLTISRLEPEDFAVYYCQQWEGNPRTFGGGTKLEIK
(Clone 52; VH)
SEQ ID NO: 497
QVQLVQSGAEVKKPGASVKVSCKASGFDIQDTYMHWVRQAPGQGLEWMGRIEPASGHIKYSP
KFQGRVTMTRDTSISTVYMELSSLRSEDTAVYYCARSGGLPDWWGQGTTVTVSS
(Clone 46; VL)
SEQ ID NO: 498
EIVLTQSPGTLSLSPGERATLSCRASSSVSYMYWYQQKPGQAPRLLIYATSNLASGIPDRFS
GSGSGTDFTLTISRLEPEDFAVYYCQQWEGNPRTFGGGTKLEIK
(Clone 46; VH)
SEQ ID NO: 499
QVQLVQSGAEVKKPGASVKVSCKASGFDIQDTYMHWVRQAPGQGLEWMGRIEPASGHVKYSP
KFQVRVTMTRDTSISTVYMELSSLRSEDTAVYYCARSGGLPDWWGQGTTVTVSS
(Clone 47; VL)
SEQ ID NO: 500
EIVLTQSPGTLSLSPGERATLSCRASSSVSYMYWYQQKPGQAPRLLIYATSNLASGIPDRFS
GSGSGTDFTLTISRLEPEDFAVYYCQQWEGNPRTFGGGTKLEIK
(Clone 47; VH)
SEQ ID NO: 501
QVQLVQSGAEVKKPGASVKVSCKASGFDIQDTYMHWVRQAPGQGLEWMGRIEPASGHVKYDP
KFQTRVTMTRDTSISTVYMELSSLRSEDTAVYYCARSGGLPDWWGQGTTVTVSS
(Clone 14; VL)
SEQ ID NO: 502
EIVLTQSPGTLSLSPGERATLSCRASSSVSYMYWYQQKPGQAPRLLIYATSNLASGIPDRFS
GSGSGTDFTLTISRLEPEDFAVYYCQQWQGNPRTFGGGTKLEIK
(Clone 14; VH)
SEQ ID NO: 503
QVQLVQSGAEVKKPGASVKVSCKASGFDIQDTYMHWVRQAPGQGLEWMGRIDPASGH1KYDP
KFQkRVTMTRDTSTSTVYMELSSLRSEDTAVYYCARSGGLPDMWGQGTTVTVSS
(Clone 16L; VL)
SEQ ID NO: 504
EIVLTQSPGTLSLSPGERATLSCRASSSVSYMYWYQQKPGQAPRLLIYATSNLASGIPDRFS
GSGSGTDFTLTISRLEPEDFAVYYCQQWQGNPRTFGGGTKLEIK
(Clone 16L; VH)
SEQ ID NO: 505
QVQLVQSGAEVKKPGASVKVSCKASGFDIQDTYMHWVRQAPGQGLEWMGRIDPASGHvKlDP
KFQVRVTMTRDTSTSTVYMELSSLRSEDTAVYYCARSGGLPDMWGQGTTVTVSS
(Clone 17L; VL)
SEQ ID NO: 506
EIVLTQSPGTLSLSPGERATLSCRASSSVSYMYWYQQKPGQAPRLLIYATSNLASGIPDRFS
GSGSGTDFTLTISRLEPEDFAVYYCQQWQGNPRTFGGGTKLEIK
(Clone 17L; VH)
SEQ ID NO: 507
QVQLVQSGAEVKKPGASVKVSCKASGFDIQDTYMHWVRQAPGQGLEWMGRIDPASGHLKYDP
KFQVRVTMTRDTSISTVYMELSSLRSEDTAVYYCARSGGLPDMWGQGTTVTVSS
(Clone 17L-1; VL)
SEQ ID NO: 508
EIVLTQSPGTLSLSPGERATLSCRASSSVSYMYWYQQKPGQAPRLLIYATSNLASGIPDRFS
GSGSGTDFTLTISRLEPEDFAVYYCQQWQGNPRTFGGGTKLEIK
(Clone 17L-1; VH)
SEQ ID NO: 509
QVQLVQSGAEVKKPGASVKVSCKASGFDIQDTYMHWVRQAPGQGLEWMGRIDPASGHLKYDP
KFQRRVTMTRDTSTSTVYMELSSLRSEDTAVYYCARSGGLPDMWGQGTTVTVSS
(Clone 23; VL)
SEQ ID NO: 510
EIVLTQSPGTLSLSPGERATLSCRASSSVSYMYWYQQKPGQAPRLLIYATSNLASGIPDRFS
GSGSGTDFTLTISRLEPEDFAVYYCQQWEGNPRTFGGGTKLEIK
(Clone 23; VH)
SEQ ID NO: 511
QVQLVQSGAEVKKPGASVKVSCKASGFDIQDTYMHWVRQAPGQGLEWMGRIDPASGHLKYDP
KFQNRVTMTRDTSTSTVYMELSSLRSEDTAVYYCARSGGLPDKWGQGTTVTVSS
(Clone A1; VL)
SEQ ID NO: 512
EIVLTQSPGTLSLSPGERATLSCGASSSVSYMYWYQQKPGQAPRLLIYATSNLASGIPDRFS
GSGSGTDFTLTISRLEPEDFAVYYCQQWEGNPRTFGGGTKLEIK
(Clone A1; VH)
SEQ ID NO: 513
QVQLVQSGAEVKKPGASVKVSCKASGFDIQDTYMHWVRQAPGQGLEWMGRIDPASGHLKYDP
KFQNRVTMTRDTSTSTVYMELSSLRSEDTAVYYCARSGGLPDKWGQGTTVTVSS
(Clone 53; VL)
SEQ ID NO: 514
EIVLTQSPGTLSLSPGERATLSCRASSSVSYMYWYQQKPGQAPRLLIYATSNLASGIPDRFS
GSGSGTDFTLTISRLEPEDFAVYYCQQWEGNPRTFGGGTKLEIK
(Clone 53; VH)
SEQ ID NO: 515
QVQLVQSGAEVKKPGASVKVSCKASGFDIQDTYMHWVRQAPGQGLEWMGRIEPASGHLKYDP
KFQERVTMTRDTSISTVYMELSSLRSEDTAVYYCARSGGLPDKWGQGTTVTVSS
(Clone E1; VL)
SEQ ID NO: 516
EIVLTQSPGTLSLSPGERATLSCGASSSVSYMYWYQQKPGQAPRLLIYATSNLASGIPDRFS
GSGSGTDFTLTISRLEPEDFAVYYCQQWEGNPRTFGGGTKLEIK
(Clone E1; VH)
SEQ ID NO: 517
QVQLVQSGAEVKKPGASVKVSCKASGFDIQDTYMHWVRQAPGQGLEWMGRIEPASGHLKYDP
KFQERVTMTRDTSISTVYMELSSLRSEDTAVYYCARSGGLPDKWGQGTTVTVSS
(Clone 3-17L V-A; VL)
SEQ ID NO: 518
EIVLTQSPGTLSLSPGERATLSCRASSSVSYMYWYQQKPGQAPRLLIYATSNLASGIPDRFS
GSGSGTDFTLTISRLEPEDFAVYYCQQWQGNPRTFGGGTKLEIK
(Clone 3-17L V-A; VH)
SEQ ID NO: 519
QVQLVQSGAEVKKPGASVKVSCKASGFDIQDTYMHWVRQAPGQGLEWMGRIDPASGHLKYDP
KFQGRVTITRDTSASTAYMELSSLRSEDTAVYYCARSGGLPDMWGQGTTVTVSS
(Clone 3-17L; VL)
SEQ ID NO: 520
EIVLTQSPGTLSLSPGERATLSCRASSSVSYMYWYQQKPGQAPRLLIYATSNLASGIPDRFS
GSGSGTDFTLTISRLEPEDFAVYYCQQWQGNPRTFGGGTKLEIK
(Clone 3-17L; VH)
SEQ ID NO: 521
QVQLVQSGAEVKKPGASVKVSCKASGFDIQDTYMHWVRQAPGQGLEWMGRIDPASGHLKYDP
KFQGRVTITRDTSASTVYMELSSLRSEDTAVYYCARSGGLPDMWGQGTTVTVSS
(Clone L8mod; VL)
SEQ ID NO: 522
EIVLTQSPGTLSLSPGERATLSCRASSSVSYMYWYQQKPGQAPRLLIYATSNLASGIPDRFS
GSGSGTDFTLTISRLEPEDFAVYYCQQWSGNPRTFGGGTKLEIK
(Clone L8mod; VIH)
SEQ ID NO: 523
QVQLVQSGAEVKKPGASVKVSCKASGFDIQDTYMHWVRQAPGQGLEWMGRIDPASGHTKYDP
KFQGRATITTDTSASTAYLQLSSLRSEDTAVYYCARSGGLPDVWGQGTTVTVSS
(Clone X-V; VL)
SEQ ID NO: 524
EIVLTQSPGTLSLSPGERATLSCRASSSVSYMYWYQQKPGQAPRLLIYATSNLASGIPDRFS
GSGSGTDFTLTISRLEPEDFAVYYCQQWSGNPRTFGGGTKLEIK
(Clone X-V; VH)
SEQ ID NO: 525
QVQLVQSGAEVKKPGASVKVSCKASGFDIQDTYMHWVRQAPGQGLEWMGRIDPASGHTKYDP
KFQVRATITTDTSASTAYLQLSSLRSEDTAVYYCARSGGLPDFWGQGTTVTVSS
(Clone X; VL)
SEQ ID NO: 526
EIVLTQSPGTLSLSPGERATLSCRASSSVSYMYWYQQKPGQAPRLLIYATSNLASGIPDRFS
GSGSGTDFTLTISRLEPEDFAVYYCQQWSGNPRTFGGGTKLEIK
(Clone X; VH)
SEQ ID NO: 527
QVQLVQSGAEVKKPGASVKVSCKASGFDIQDTYMHWVRQAPGQGLEWMGRIDPASGHTKYDP
KFQGRATITTDTSASTAYLQLSSLRSEDTAVYYCARSGGLPDFWGQGTTVTVSS
(Clone H3-1; VL)
SEQ ID NO: 528
EIVLTQSPGTLSLSPGERATLSCRASSSVSYMYWYQQKPGQAPRLLIYATSNLASGIPDRFS
GSGSGTDFTLTISRLEPEDFAVYYCQQWSGNPRTFGGGTKLEIK
(Clone H3-1; VH)
SEQ ID NO: 529
QVQLVQSGAEVKKPGASVKVSCKASGFDIQDTYMHWVRQAPGQGLEWMGRIDPASGHTKYDP
KFQGRATITTDTSASTAYLQLSSLRSEDTAVYYCARSGGLPDLWGQGTTVTVSS
(Clone XL3-6; VL)
SEQ ID NO: 530
EIVLTQSPGTLSLSPGERATLSCRASSSVSYMYWYQQKPGQAPRLLIYATSNLASGIPDRFS
GSGSGTDFTLTISRLEPEDFAVYYCSQWSGNPRTFGGGTKLEIK
(Clone XL3-6; VH)
SEQ ID NO: 531
QVQLVQSGAEVKKPGASVKVSCKASGFDIQDTYMHWVRQAPGQGLEWMGRIDPASGHTKYDP
KFQGRATITTDTSASTAYLQLSSLRSEDTAVYYCARSGGLPDFWGQGTTVTVSS
(Clone XL3-10; VL)
SEQ ID NO: 532
EIVLTQSPGTLSLSPGERATLSCRASSSVSYMYWYQQKPGQAPRLLIYATSNLASGIPDRFS
GSGSGTDFTLTISRLEPEDFAVYYCQQWSGNPRSFGGGTKLEIK
(Clone XL3-10; VH)
SEQ ID NO: 533
QVQLVQSGAEVKKPGASVKVSCKASGFDIQDTYMHWVRQAPGQGLEWMGRIDPASGHTKYDP
KFQGRATITTDTSASTAYLQLSSLRSEDTAVYYCARSGGLPDFWGQGTTVTVSS
(Clone XL3-15; VL)
SEQ ID NO: 534
EIVLTQSPGTLSLSPGERATLSCRASSSVSYMYWYQQKPGQAPRLLIYATSNLASGIPDRFS
GSGSGTDFTLTISRLEPEDFAVYYCQQWSRNPRTFGGGTKLEIK
(Clone XL3-15; VH)
SEQ ID NO: 535
QVQLVQSGAEVKKPGASVKVSCKASGFDIQDTYMHWVRQAPGQGLEWMGRIDPASGHTKYDP
KFQGRATITTDTSASTAYLQLSSLRSEDTAVYYCARSGGLPDFWGQGTTVTVSS
(Clone L3-13; VL)
SEQ ID NO: 536
EIVLTQSPGTLSLSPGERATLSCRASSSVSYMYWYQQKPGQAPRLLIYATSNLASGIPDRFS
GSGSGTDFTLTISRLEPEDFAVYYCQQWKGNPRTFGGGTKLEIK
(Clone L3-13; VH)
SEQ ID NO: 537
QVQLVQSGAEVKKPGASVKVSCKASGFDIQDTYMHWVRQAPGQGLEWMGRIDPASGHTKYDP
KFQGRATITTDTSASTAYLQLSSLRSEDTAVYYCARSGGLPDFWGQGTTVTVSS
(Clone 112-2; VL)
SEQ ID NO: 538
EIVLTQSPGTLSLSPGERATLSCRASSSVSYMYWYQQKPGQAPRLLIYATSNLASGIPDRFS
GSGSGTDFTLTISRLEPEDFAVYYCQQWSGNPRTFGGGTKLEIK
(Clone 112-2; VH)
SEQ ID NO: 539
QVQLVQSGAEVKKPGASVKVSCKASGFDIQDTYMHWVRQAPGQGLEWMGRIDPASGHSKYDP
KFQVRATITTDTSASTAYLQLSSLRSEDTAVYYCARSGGLPDFWGQGTTVTVSS
(Clone 112-5; VL)
SEQ ID NO: 540
EIVLTQSPGTLSLSPGERATLSCRASSSVSYMYWYQQKPGQAPRLLIYATSNLASGIPDRFS
GSGSGTDFTLTISRLEPEDFAVYYCQQWSGNPRTFGGGTKLEIK
(Clone 112-5; VH)
SEQ ID NO: 541
QVQLVQSGAEVKKPGASVKVSCKASGFDIQDTYMHWVRQAPGQGLEWMGRIDPASGHYKYDP
KFQVRATITTDTSASTAYLQLSSLRSEDTAVYYCARSGGLPDFWGQGTTVTVSS
modified G1
SEQ ID NO: 542
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAAGAPSVF
LFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
G2 constant domains
SEQ ID NO: 543
ASTKGPSVFPLAPCSRSTSESTAALGCLVKDYFPEPVTVSWNSGALTSGVHTFPAVLQSSGL
YSLSSVVTVPSSNFGTQTYTCNVDHKPSNTKVDKTVERKCCVECPPCPAPPVAGPSVFLFPP
KPKDTLMISRTPEVTCVVVDVSHEDPEVQFNWYVDGVEVHNAKTKPREEQFNSTFRVVSVLT
VVHQDWLNGKEYKCKVSNKGLPAPIEKTISKTKGQPREPQVYTLPPSREEMTKNQVSLTCLV
KGFYPSDIAVEWESNGQPENNYKTTPPMLDSDGSFFLYSKLTVDKSRWQQGNVFSCSVMHEA
LHNHYTQKSLSLSPGK
Kappa constant domain
SEQ ID NO: 544
RTVAAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC
(L8 HFR1)
SEQ ID NO: 545
QVQLVQSGAEVKKPGASVKVSCKAS
(L8 HFR2)
SEQ ID NO: 546
WVRQAPGQGLEWMG
(L8 HFR3)
SEQ ID NO: 547
RVTMTRDTSTSTVYMELSSLRSEDTAVYYC
(L8 HFR4)
SEQ ID NO: 548
WGQGTTVTVSS
(L8 LFR1)
SEQ ID NO: 549
EIVLTQSPGTLSLSPGERATLSC
(L8 LFR2)
SEQ ID NO: 550
WYQQKPGQAPRLLIY
(L8 LFR3)
SEQ ID NO: 551
GIPDRFSGSGSGTDFTLTISRLEPEDFAVYYC
(L8 LFR4)
SEQ ID NO: 552
FGGGTKLEIK
(VH FR3)
SEQ ID NO: 586
RAT ITTDTSASTAYLQLSSLRSEDTAVYYC
(VH FR3)
SEQ ID NO: 587
RVTITRDTSASTVYMELSSLRSEDTAVYYC
(VH FR3)
SEQ ID NO: 588
RVTITRDTSASTAYMELSSLRSEDTAVYYC

1-48. (canceled)

49. A method of preparing an inflammatory bowel disease (IBD), Crohn's disease, or colitis treatment, comprising incubating a cell comprising a nucleic acid encoding an antibody or antigen-binding fragment that specifically binds tumor necrosis factor ligand 1A (TL1A) in a culture medium under conditions sufficient to secrete the antibody or antigen-binding fragment into the culture medium.

50. The method of claim 49, wherein the cell is a eukaryotic cell.

51. The method of claim 49, wherein the cell is a Chinese Hamster Ovary (CHO) cell.

52. The method of claim 49, wherein the cell is a COS cell, a HeLa cell, an L cell, or a multiple myeloma cell.

53. The method of claim 49, wherein the cell is a C127 cell, a 3T3 cell, or a BHK cell.

54. The method of claim 49, wherein the antibody or antigen-binding fragment that specifically binds TL1A comprises a heavy chain variable region comprising: an HCDR1 comprising an amino acid sequence set forth by SEQ ID NO: 553; an HCDR2 comprising an amino acid sequence set forth by any one of SEQ ID NOs: 554 to 564 or 574 to 577; and an HCDR3 comprising an amino acid sequence set forth by any one of SEQ ID NOs: 565 to 568 or 578 to 581; and a light chain variable region comprising: an LCDR1 comprising an amino acid sequence set forth by any one of SEQ ID NOs: 569 or 570; an LCDR2 comprising an amino acid sequence set forth by SEQ ID NO: 488; and an LCDR3 comprising an amino acid sequence set forth by any one of SEQ ID NOs: 571 to 573 or 582 to 585.

55. The method of claim 49, wherein the antibody or antigen-binding fragment that specifically binds TL1A comprises: a heavy chain variable region comprising an amino acid sequence at least about 90% identical to any one of SEQ ID NOs: 491, 493, 495, 497, 499, 501, 503, 505, 507, 509, 511, 513, 515, 517, 519, 521, 523, 525, 527, 529, 531, 533, 535, 537, 539, or 541; and a light chain variable region comprising an amino acid sequence at least about 90% identical to any one of SEQ ID NOs: 490, 492, 494, 496, 498, 500, 502, 504, 506, 508, 510, 512, 514, 516, 518, 520, 522, 524, 526, 528, 530, 532, 534, 536, 538, or 540.

56. The method of claim 49, wherein the antibody or antigen-binding fragment that specifically binds TL1A comprises a heavy chain variable region comprising: an HCDR1, an HCDR2, and an HCDR3 selected from any one of SEQ ID NOs: 491, 493, 495, 497, 499, 501, 503, 505, 507, 509, 511, 513, 515, 517, 519, 521, 523, 525, 527, 529, 531, 533, 535, 537, 539, or 541; and a light chain variable region comprising an LCDR1, an LCDR2, and an LCDR3 selected from any one of SEQ ID NOs: 490, 492, 494, 496, 498, 500, 502, 504, 506, 508, 510, 512, 514, 516, 518, 520, 522, 524, 526, 528, 530, 532, 534, 536, 538, or 540, wherein the CDRs are defined by the Kabat, Chothia, or IMGT method or a combination thereof.

57. The method of claim 49, further comprising subjecting the culture medium to at least one purification step.

58. The method of claim 57, wherein the purification step comprises high-performance liquid chromatography (HPLC) purification, column chromatography, or gel electrophoresis, or any combination thereof.

59. The method of claim 57, wherein the purification step comprises immunoadsorption, immunoaffinity chromatography, ammonium sulfate precipitation, or gel electrophoresis, or any combination thereof.

60. A method of preparing an inflammatory bowel disease (IBD), Crohn's disease, or colitis treatment, the method comprising admixing an antibody or antigen-binding fragment that specifically binds tumor necrosis factor ligand 1A (TL1A) and a pharmaceutically acceptable excipient, carrier, or diluent.

61. The method of claim 60, wherein the antibody or antigen-binding fragment that specifically binds TL1A comprises a heavy chain variable region comprising: an HCDR1 comprising an amino acid sequence set forth by SEQ ID NO: 553; an HCDR2 comprising an amino acid sequence set forth by any one of SEQ ID NOs: 554 to 564 or 574 to 577; and an HCDR3 comprising an amino acid sequence set forth by any one of SEQ ID NOs: 565 to 568 or 578 to 581; and a light chain variable region comprising: an LCDR1 comprising an amino acid sequence set forth by any one of SEQ ID NOs: 569 or 570; an LCDR2 comprising an amino acid sequence set forth by SEQ ID NO: 488; and an LCDR3 comprising an amino acid sequence set forth by any one of SEQ ID NOs: 571 to 573 or 582 to 585.

62. The method of claim 60, wherein the antibody or antigen-binding fragment that specifically binds TL1A comprises: a heavy chain variable region comprising an amino acid sequence at least about 90% identical to any one of SEQ ID NOs: 491, 493, 495, 497, 499, 501, 503, 505, 507, 509, 511, 513, 515, 517, 519, 521, 523, 525, 527, 529, 531, 533, 535, 537, 539, or 541; and a light chain variable region comprising an amino acid sequence at least about 90% identical to any one of SEQ ID NOs: 490, 492, 494, 496, 498, 500, 502, 504, 506, 508, 510, 512, 514, 516, 518, 520, 522, 524, 526, 528, 530, 532, 534, 536, 538, or 540.

63. The method of claim 60, wherein the antibody or antigen-binding fragment that specifically binds TL1A comprises a heavy chain variable region comprising: an HCDR1, an HCDR2, and an HCDR3 selected from any one of SEQ ID NOs: 491, 493, 495, 497, 499, 501, 503, 505, 507, 509, 511, 513, 515, 517, 519, 521, 523, 525, 527, 529, 531, 533, 535, 537, 539, or 541; and a light chain variable region comprising an LCDR1, an LCDR2, and an LCDR3 selected from any one of SEQ ID NOs: 490, 492, 494, 496, 498, 500, 502, 504, 506, 508, 510, 512, 514, 516, 518, 520, 522, 524, 526, 528, 530, 532, 534, 536, 538, or 540, wherein the CDRs are defined by the Kabat, Chothia, or IMGT method or a combination thereof.


SEQUENCE LISTING (694 sequences)

SEQ ID NO: 1 - DNA, 348 bp, synthetic construct
  misc_feature 1..348: note = Description of Artificial Sequence: Syntheticpolynucleotide
  source 1..348: mol_type = other DNA
gaagttcagctgcaacagtctggcgccgagctggttaagcctggcgcttctgtgaagctgagctgtaccgcctctggcttcgacatccaagacacctacatgcactgggtcaagcagaggcctgagcagggactcgagtggatcggcagaattgatcctgccagcggccacaccaaatacgaccccaagttccaagtgaaggccaccatcaccaccgacaccagcagcaataccgcctacctgcagctgagcagcctgacctctgaagataccgccgtgtactactgcagcagatctggcggactgcccgatgtttggggagccggaacaaccgtgacagtgtccagc

SEQ ID NO: 2 - DNA, 348 bp, synthetic construct
  misc_feature 1..348: note = Description of Artificial Sequence: Syntheticpolynucleotide
  source 1..348: mol_type = other DNA
gaggttcaacttcaacaatcgggggccgagctggttaagcccggcgcttctgtaaaattgtcttgcactgcctctgggtttgacatccaagatacatatatgcattgggtgaaacagcgtcccgagcagggcttggagtggattggacgtattgaccccgcctctgggcacacgaaatatgatcctaagttccaggttaaagcgactatcacaacggacacctccagcaatacggcttatttacagttatcctcgctgacctctgaggatactgcagtgtactactgctctcgctctggtggtctgccagacgtgtggggtgcaggaactacagttactgtgtcttca

SEQ ID NO: 3 - protein, 116 aa, synthetic construct
  REGION 1..116: note = Description of Artificial Sequence: Syntheticpolypeptide
EVQLQQSGAELVKPGASVKLSCTASGFDIQDTYMHWVKQRPEQGLEWIGRIDPASGHTKYDPKFQVKATITTDTSSNTAYLQLSSLTSEDTAVYYCSRSGGLPDVWGAGTTVTVSS

SEQ ID NO: 4 - DNA, 318 bp, synthetic construct
  misc_feature 1..318: note = Description of Artificial Sequence: Syntheticpolynucleotide
  source 1..318: mol_type = other DNA
caaattgtgctgtctcagagccccgccatcctgagtgcttctccaggcgagaaagtgaccatgacctgcagagccagcagcagcgtgtcctacatgtactggtatcagcagaagcccggcagcagccccaagccttggatctacgccacaagcaatctggccagcggcgtgcccgatagattttctggctctggcagcggcaccagctacagcctgacaatctctagagtggaagccgaggatgccgccacctactactgtcaacagtggagcggcaaccccagaacctttggcggaggcaccaagctggaaatcaag

SEQ ID NO: 5 - DNA, 318 bp, synthetic construct
  misc_feature 1..318: note = Description of Artificial Sequence: Syntheticpolynucleotide
  source 1..318: mol_type = other DNA
caaatcgtcctgtcacagtccccggcgatcctttctgcttcaccaggagagaaggtaaccatgacatgtcgcgcctcttcctcagtttcttacatgtactggtaccagcagaaaccaggatcatctcccaaaccctggatctacgctacatcaaaccttgcatctggcgtgccagaccgtttttcagggtcgggctcggggacttcctattcattaaccatttctcgcgtagaagcggaagacgccgccacgtattattgtcagcagtggtcaggaaatccgcgcacattcggaggcggaacgaaattggagatcaaa

SEQ ID NO: 6 - protein, 106 aa, synthetic construct
  REGION 1..106: note = Description of Artificial Sequence: Syntheticpolypeptide
QIVLSQSPAILSASPGEKVTMTCRASSSVSYMYWYQQKPGSSPKPWIYATSNLASGVPDRFSGSGSGTSYSLTISRVEAEDAATYYCQQWSGNPRTFGGGTKLEIK

SEQ ID NO: 7 - DNA, 30 bp, synthetic construct
  misc_feature 1..30: note = Description of Artificial Sequence: Syntheticoligonucleotide
  source 1..30: mol_type = other DNA
ggcttcgacatccaagacacctacatgcac

SEQ ID NO: 8 - DNA, 30 bp, synthetic construct
  misc_feature 1..30: note = Description of Artificial Sequence: Syntheticoligonucleotide
  source 1..30: mol_type = other DNA
gggtttgacatccaagatacatatatgcat

SEQ ID NO: 9 - protein, 10 aa, synthetic construct
  REGION 1..10: note = Description of Artificial Sequence: Syntheticpeptide
GFDIQDTYMH

SEQ ID NO: 10 - DNA, 51 bp, synthetic construct
  misc_feature 1..51: note = Description of Artificial Sequence: Syntheticoligonucleotide
  source 1..51: mol_type = other DNA
agaattgatcctgccagcggccacaccaaatacgaccccaagttccaagtg

SEQ ID NO: 11 - DNA, 51 bp, synthetic construct
  misc_feature 1..51: note = Description of Artificial Sequence: Syntheticoligonucleotide
  source 1..51: mol_type = other DNA
cgtattgaccccgcctctgggcacacgaaatatgatcctaagttccaggtt

SEQ ID NO: 12 - protein, 17 aa, synthetic construct
  REGION 1..17: note = Description of Artificial Sequence: Syntheticpeptide
  VARIANT 2: note = Ile or Leu
  VARIANT 3: note = Asp or Glu
  VARIANT 5: note = Ala or Glu
  VARIANT 6: note = Gly or Ser
  VARIANT 7: note = Ala or Gly
  VARIANT 9: note = Ile, Leu, Thr, or Val
  VARIANT 11: note = Ile, Leu, Met, Ser, Thr, Val, or Tyr
  VARIANT 12: note = Asp, Ile, Asn, Arg, or Ser
  VARIANT 16: note = Gln or Arg
  VARIANT 17: note = Ala, Asp, Glu, Gly, His, Lys, Leu, Met, Asn, Pro, Arg,Ser, Thr, or Val
RXXPXXXHXKXXPKFXX

SEQ ID NO: 13 - DNA, 21 bp, synthetic construct
  misc_feature 1..21: note = Description of Artificial Sequence: Syntheticoligonucleotide
  source 1..21: mol_type = other DNA
tctggcggactgcccgatgtt

SEQ ID NO: 14 - DNA, 21 bp, synthetic construct
  misc_feature 1..21: note = Description of Artificial Sequence: Syntheticoligonucleotide
  source 1..21: mol_type = other DNA
tctggtggtctgccagacgtg

SEQ ID NO: 15 - protein, 7 aa, synthetic construct
  REGION 1..7: note = Description of Artificial Sequence: Syntheticpeptide
SGGLPDV

SEQ ID NO: 16 - DNA, 30 bp, synthetic construct
  misc_feature 1..30: note = Description of Artificial Sequence: Syntheticoligonucleotide
  source 1..30: mol_type = other DNA
agagccagcagcagcgtgtcctacatgtac

SEQ ID NO: 17 - DNA, 30 bp, synthetic construct
  misc_feature 1..30: note = Description of Artificial Sequence: Syntheticoligonucleotide
  source 1..30: mol_type = other DNA
cgcgcctcttcctcagtttcttacatgtac

SEQ ID NO: 18 - protein, 10 aa, synthetic construct
  REGION 1..10: note = Description of Artificial Sequence: Syntheticpeptide
  VARIANT 1: note = Gly, Arg, or Trp
  VARIANT 7: note = Ile or Ser
  VARIANT 8: note = Phe or Tyr
  VARIANT 9: note = Leu or Met
  VARIANT 10: note = Arg or Tyr
XASSSVXXXX

SEQ ID NO: 19 - DNA, 21 bp, synthetic construct
  misc_feature 1..21: note = Description of Artificial Sequence: Syntheticoligonucleotide
  source 1..21: mol_type = other DNA
gccacaagcaatctggccagc

SEQ ID NO: 20 - DNA, 21 bp, synthetic construct
  misc_feature 1..21: note = Description of Artificial Sequence: Syntheticoligonucleotide
  source 1..21: mol_type = other DNA
gctacatcaaaccttgcatct

SEQ ID NO: 21 - skipped

SEQ ID NO: 22 - DNA, 27 bp, synthetic construct
  misc_feature 1..27: note = Description of Artificial Sequence: Syntheticoligonucleotide
  source 1..27: mol_type = other DNA
caacagtggagcggcaaccccagaacc

SEQ ID NO: 23 - DNA, 27 bp, synthetic construct
  misc_feature 1..27: note = Description of Artificial Sequence: Syntheticoligonucleotide
  source 1..27: mol_type = other DNA
cagcagtggtcaggaaatccgcgcaca

SEQ ID NO: 24 - protein, 9 aa, synthetic construct
  REGION 1..9: note = Description of Artificial Sequence: Syntheticpeptide
QQWSGNPRT

SEQ ID NO: 25 - DNA, 348 bp, synthetic construct
  misc_feature 1..348: note = Description of Artificial Sequence: Syntheticpolynucleotide
  source 1..348: mol_type = other DNA
caagtacaattagtccagtcgggtgccgaggtaaaaaaacctggagcatccgtaaaactgtcttgcaaagcatcggggtttgacatccaggacacctacatgcactgggtgcgtcaagctccaggacagggattagagtggatgggtcgcatcgaccccgcgagcggacacacgaaatacgaccctaaatttcaagtacgtgtcacgatgactaccgacactagtacgagcactgtttatatggaattgtcctcgttacgctcagaggatacggcagtctattattgcagccgttccggaggcttacccgacgtctggggacagggaactactgtaacagtcagtagt

SEQ ID NO: 26 - protein, 116 aa, synthetic construct
  REGION 1..116: note = Description of Artificial Sequence: Syntheticpolypeptide
QVQLVQSGAEVKKPGASVKLSCKASGFDIQDTYMHWVRQAPGQGLEWMGRIDPASGHTKYDPKFQVRVTMTTDTSTSTVYMELSSLRSEDTAVYYCSRSGGLPDVWGQGTTVTVSS

SEQ ID NO: 27 - DNA, 318 bp, synthetic construct
  misc_feature 1..318: note = Description of Artificial Sequence: Syntheticpolynucleotide
  source 1..318: mol_type = other DNA
gagattgtgttaacgcaatcaccggggactttatcgctgtcgccgggggagcgcgttacaatgtcttgtcgcgcttcctcttcggtttcatacatgtattggtatcaacaaaaaccgggacaggctccacgcccctggatttacgctactagcaatttggcctcgggcgttcccgaccgcttcagcgggtcagggagcggcaccgattacacgttgaccatctctcgtctggaacctgaagacttcgcggtctattactgtcaacaatggtcgggaaatccccgtacatttggcggagggacgaagttggaaattaaa

SEQ ID NO: 28 - protein, 106 aa, synthetic construct
  REGION 1..106: note = Description of Artificial Sequence: Syntheticpolypeptide
EIVLTQSPGTLSLSPGERVTMSCRASSSVSYMYWYQQKPGQAPRPWIYATSNLASGVPDRFSGSGSGTDYTLTISRLEPEDFAVYYCQQWSGNPRTFGGGTKLEIK

SEQ ID NO: 29 - DNA, 30 bp, synthetic construct
  misc_feature 1..30: note = Description of Artificial Sequence: Syntheticoligonucleotide
  source 1..30: mol_type = other DNA
gggtttgacatccaggacacctacatgcac

SEQ ID NO: 30 - DNA, 51 bp, synthetic construct
  misc_feature 1..51: note = Description of Artificial Sequence: Syntheticoligonucleotide
  source 1..51: mol_type = other DNA
cgcatcgaccccgcgagcggacacacgaaatacgaccctaaatttcaagta

SEQ ID NO: 31 - DNA, 21 bp, synthetic construct
  misc_feature 1..21: note = Description of Artificial Sequence: Syntheticoligonucleotide
  source 1..21: mol_type = other DNA
tccggaggcttacccgacgtc

SEQ ID NO: 32 - DNA, 36 bp, synthetic construct
  misc_feature 1..36: note = Description of Artificial Sequence: Syntheticoligonucleotide
  source 1..36: mol_type = other DNA
cgcgcttcctcttcggtttcatacatgtattggtat

SEQ ID NO: 33 - DNA, 21 bp, synthetic construct
  misc_feature 1..21: note = Description of Artificial Sequence: Syntheticoligonucleotide
  source 1..21: mol_type = other DNA
gctactagcaatttggcctcg

SEQ ID NO: 34 - DNA, 27 bp, synthetic construct
  misc_feature 1..27: note = Description of Artificial Sequence: Syntheticoligonucleotide
  source 1..27: mol_type = other DNA
caacaatggtcgggaaatccccgtaca

SEQ ID NO: 35 - DNA, 348 bp, synthetic construct
  misc_feature 1..348: note = Description of Artificial Sequence: Syntheticpolynucleotide
  source 1..348: mol_type = other DNA
caagtacaattagtccagtcgggtgccgaggtaaaaaaacctggagcatccgtaaaactgtcttgcaaagcatcggggtttgacatccaggacacctacatgcactgggtgcgtcaagctccaggacagggattagagtggatgggtcgcatcgaccccgcgagcggacacacgaaatacgaccctaaatttcaagtacgtgtcacgatgactcgtgacactagtacgagcactgtttatatggaattgtcctcgttacgctcagaggatacggcagtctattattgcagccgttccggaggcttacccgacgtctggggacagggaactactgtaacagtcagtagt

SEQ ID NO: 36 - protein, 116 aa, synthetic construct
  REGION 1..116: note = Description of Artificial Sequence: Syntheticpolypeptide
QVQLVQSGAEVKKPGASVKLSCKASGFDIQDTYMHWVRQAPGQGLEWMGRIDPASGHTKYDPKFQVRVTMTRDTSTSTVYMELSSLRSEDTAVYYCSRSGGLPDVWGQGTTVTVSS

SEQ ID NO: 37 - DNA, 318 bp, synthetic construct
  misc_feature 1..318: note = Description of Artificial Sequence: Syntheticpolynucleotide
  source 1..318: mol_type = other DNA
gagattgtgttaacgcaatcaccggggactttatcgctgtcgccgggggagcgcgcgacactgtcttgtcgcgcttcctcttcggtttcatacatgtattggtatcaacaaaaaccgggacaggctccacgcctgctgatttacgctactagcaatttggcctcgggcatccccgaccgcttcagcgggtcagggagcggcaccgattttacgttgaccatctctcgtctggaacctgaagacttcgcggtctattactgtcaacaatggtcgggaaatccccgtacatttggcggagggacgaagttggaaattaaa

SEQ ID NO: 38 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDFTLTISRLEPEDFAVYYCQQWSGNPRTFGGGTKLEIK

SEQ ID NO: 39 - DNA, 348 bp, synthetic construct
  misc_feature 1..348: note = Description of Artificial Sequence: Syntheticpolynucleotide
  source 1..348: mol_type = other DNA
caagtacaattagtccagtcgggtgccgaggtaaaaaaacctggagcatccgtaaaagtctcttgcaaagcatcggggtttgacatccaggacacctacatgcactgggtgcgtcaagctccaggacagggattagagtggatgggtcgcatcgaccccgcgagcggacacacgaaatacgaccctaaatttcaagtacgtgtcacgatgactcgtgacactagtacgagcactgtttatatggaattgtcctcgttacgctcagaggatacggcagtctattattgcgcacgttccggaggcttacccgacgtctggggacagggaactactgtaacagtcagtagt

SEQ ID NO: 40 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDVWGQGTTVTVSS

SEQ ID NO: 41 - DNA, 318 bp, synthetic construct
  misc_feature 1..318: note = Description of Artificial Sequence: Syntheticpolynucleotide
  source 1..318: mol_type = other DNA
gagattgtgttaacgcaatcaccggggactttatcgctgtcgccgggggagcgcgcgacactgtcttgtcgcgcttcctcttcggtttcatacatgtattggtatcaacaaaaaccgggacaggctccacgcctgctgatttacgctactagcaatttggcctcgggcgttcccgaccgcttcagcgggtcagggagcggcaccgattacacgttgaccatctctcgtctggaacctgaagacttcgcggtctattactgtcaacaatggtcgggaaatccccgtacatttggcggagggacgaagttggaaattaaa

SEQ ID NO: 42 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGVPDRFSGSGSGTDYTLTISRLEPEDFAVYYCQQWSGNPRTFGGGTKLEIK

SEQ ID NO: 43 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDKWGQGTTVTVSS

SEQ ID NO: 44 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDMWGQGTTVTVSS

SEQ ID NO: 45 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDQWGQGTTVTVSS

SEQ ID NO: 46 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDWWGQGTTVTVSS

SEQ ID NO: 47 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDFTLTISRLEPEDFAVYYCQQWDGNPRTFGGGTKLEIK

SEQ ID NO: 48 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDFTLTISRLEPEDFAVYYCQQWEGNPRTFGGGTKLEIK

SEQ ID NO: 49 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDFTLTISRLEPEDFAVYYCQQWHGNPRTFGGGTKLEIK

SEQ ID NO: 50 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDFTLTISRLEPEDFAVYYCQQWNGNPRTFGGGTKLEIK

SEQ ID NO: 51 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDFTLTISRLEPEDFAVYYCQQWQGNPRTFGGGTKLEIK

SEQ ID NO: 52 - protein, 116 aa, synthetic construct
  REGION 1..116: note = Description of Artificial Sequence: Syntheticpolypeptide
  VARIANT 28: note = Asp or Glu
  VARIANT 29: note = Ile, Pro, or Val
  VARIANT 30: note = Gly, Gln, Ser, or Val
  VARIANT 33: note = Phe or Tyr
  VARIANT 34: note = Ile or Met
  VARIANT 102: note = Leu or Met
  VARIANT 105: note = Glu, Ile, Lys, Leu, Met, Gln, Thr, Val, Trp, or Tyr
QVQLVQSGAEVKKPGASVKVSCKASGFXXXDTXXHWVRQAPGQGLEWMGRIDPASGHTKYDPKFQVRVTMTRDTSTSTVYMELSSLRSEDTAVYYCARSGGXPDXWGQGTTVTVSS

SEQ ID NO: 53 - protein, 106 aa, synthetic construct
  REGION 1..106: note = Description of Artificial Sequence: Syntheticpolypeptide
  VARIANT 88: note = Gln or Asn
  VARIANT 91: note = Asp, Glu, His, Asn, Gln, or Ser
  VARIANT 92: note = Ala or Gly
  VARIANT 93: note = Asp, Phe, Lys, Asn, Arg, Ser, or Thr
EIVLTQSPGTLSLSPGERATLSCRASSSVSYMYWYQQKPGQAPRLLIYATSNLASGIPDRFSGSGSGTDFTLTISRLEPEDFAVYYCXQWXXXPRTFGGGTKLEIK

SEQ ID NO: 54 - protein, 116 aa, synthetic construct
  REGION 1..116: note = Description of Artificial Sequence: Syntheticpolypeptide
  VARIANT 28: note = Asp or Glu
  VARIANT 29: note = Ile, Pro, or Val
  VARIANT 30: note = Gly, Gln, Ser, or Val
  VARIANT 33: note = Phe or Tyr
  VARIANT 34: note = Ile or Met
  VARIANT 102: note = Leu or Met
  VARIANT 105: note = Glu, Ile, Lys, Leu, Met, Gln, Thr, Val, Trp, or Tyr
QVQLVQSGAEVKKPGASVKVSCKASGFXXXDTXXHWVRQAPGQGLEWMGRIDPASGHTKYDPKFQVRVTMTRDTSTSTVYMELSSLRSEDTAVYYCSRSGGXPDXWGQGTTVTVSS

SEQ ID NO: 55-75 - skipped (21 entries)

SEQ ID NO: 76 - protein, 9 aa, synthetic construct
  REGION 1..9: note = Description of Artificial Sequence: Syntheticpeptide
QQWSGTPRT

SEQ ID NO: 77 - skipped

SEQ ID NO: 78 - protein, 9 aa, synthetic construct
  REGION 1..9: note = Description of Artificial Sequence: Syntheticpeptide
QQWSGDPRT

SEQ ID NO: 79 - skipped

SEQ ID NO: 80 - protein, 9 aa, synthetic construct
  REGION 1..9: note = Description of Artificial Sequence: Syntheticpeptide
QQWSGFPRT

SEQ ID NO: 81 - skipped

SEQ ID NO: 82 - protein, 9 aa, synthetic construct
  REGION 1..9: note = Description of Artificial Sequence: Syntheticpeptide
QQWSGKPRT

SEQ ID NO: 83 - skipped

SEQ ID NO: 84 - protein, 9 aa, synthetic construct
  REGION 1..9: note = Description of Artificial Sequence: Syntheticpeptide
QQWSGRPRT

SEQ ID NO: 85 - skipped

SEQ ID NO: 86 - protein, 9 aa, synthetic construct
  REGION 1..9: note = Description of Artificial Sequence: Syntheticpeptide
QQWSGSPRT

SEQ ID NO: 87-149 - skipped (63 entries)

SEQ ID NO: 150 - protein, 10 aa, synthetic construct
  REGION 1..10: note = Description of Artificial Sequence: Syntheticpeptide
  VARIANT 3: note = Asp or Glu
  VARIANT 4: note = Ile, Leu, Pro, or Val
  VARIANT 5: note = Gly, Gln, Ser, or Val
  VARIANT 7: note = Ala, Ser, or Thr
  VARIANT 8: note = Phe or Tyr
  VARIANT 9: note = Ile, Leu, or Met
GFXXXDXXXH

SEQ ID NO: 151-483 - skipped (333 entries)

SEQ ID NO: 484 - protein, 5 aa, synthetic construct
  REGION 1..5: note = Description of Artificial Sequence: Syntheticpeptide
DTYMH

SEQ ID NO: 485 - protein, 5 aa, synthetic construct
  REGION 1..5: note = Description of Artificial Sequence: Syntheticpeptide
PASGH

SEQ ID NO: 486 - protein, 6 aa, synthetic construct
  REGION 1..6: note = Description of Artificial Sequence: Syntheticpeptide
SGGLPD

SEQ ID NO: 487 - protein, 9 aa, synthetic construct
  REGION 1..9: note = Description of Artificial Sequence: Syntheticpeptide
ASSSVSYMY

SEQ ID NO: 488 - protein, 7 aa, synthetic construct
  REGION 1..7: note = Description of Artificial Sequence: Syntheticpeptide
ATSNLAS

SEQ ID NO: 489 - protein, 5 aa, synthetic construct
  REGION 1..5: note = Description of Artificial Sequence: Syntheticpeptide
GNPRT

SEQ ID NO: 490 - protein, 106 aa, synthetic construct; identical to SEQ ID NO: 38
  REGION 1..106: note = Description of Artificial Sequence: Syntheticpolypeptide

SEQ ID NO: 491 - protein, 116 aa, synthetic construct; identical to SEQ ID NO: 40
  REGION 1..116: note = Description of Artificial Sequence: Syntheticpolypeptide

SEQ ID NO: 492 - protein, 106 aa, synthetic construct; identical to SEQ ID NO: 48
  REGION 1..106: note = Description of Artificial Sequence: Syntheticpolypeptide

SEQ ID NO: 493 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYDPKFQGRVTMTRDTSTSTVYMELSSLRSEDTAVYYCARSGGLPDWWGQGTTVTVSS

SEQ ID NO: 494 - protein, 106 aa, synthetic construct; identical to SEQ ID NO: 48
  REGION 1..106: note = Description of Artificial Sequence: Syntheticpolypeptide

SEQ ID NO: 495 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYSPKFQGRVTMTRDTSTSTVYMELSSLRSEDTAVYYCARSGGLPDWWGQGTTVTVSS

SEQ ID NO: 496 - protein, 106 aa, synthetic construct
  REGION 1..106: note = Description of Artificial Sequence: Syntheticpolypeptide
EIVLTQSPGTLSLSPGERATLSCGASSSVSYMYWYQQKPGQAPRLLIYATSNLASGIPDRFSGSGSGTDFTLTISRLEPEDFAVYYCQQWEGNPRTFGGGTKLEIK

SEQ ID NO: 497 - protein, 116 aa, synthetic construct; identical to SEQ ID NO: 495
  REGION 1..116: note = Description of Artificial Sequence: Syntheticpolypeptide

SEQ ID NO: 498 - protein, 106 aa, synthetic construct; identical to SEQ ID NO: 48
  REGION 1..106: note = Description of Artificial Sequence: Syntheticpolypeptide

SEQ ID NO: 499 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHVKYSPKFQVRVTMTRDTSTSTVYMELSSLRSEDTAVYYCARSGGLPDWWGQGTTVTVSS

SEQ ID NO: 500 - protein, 106 aa, synthetic construct; identical to SEQ ID NO: 48
  REGION 1..106: note = Description of Artificial Sequence: Syntheticpolypeptide

SEQ ID NO: 501 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHVKYDPKFQTRVTMTRDTSTSTVYMELSSLRSEDTAVYYCARSGGLPDWWGQGTTVTVSS

SEQ ID NO: 502 - protein, 106 aa, synthetic construct; identical to SEQ ID NO: 51
  REGION 1..106: note = Description of Artificial Sequence: Syntheticpolypeptide

SEQ ID NO: 503 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHIKYDPKFQKRVTMTRDTSTSTVYMELSSLRSEDTAVYYCARSGGLPDMWGQGTTVTVSS

SEQ ID NO: 504 - protein, 106 aa, synthetic construct; identical to SEQ ID NO: 51
  REGION 1..106: note = Description of Artificial Sequence: Syntheticpolypeptide

SEQ ID NO: 505 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHVKIDPKFQVRVTMTRDTSTSTVYMELSSLRSEDTAVYYCARSGGLPDMWGQGTTVTVSS

SEQ ID NO: 506 - protein, 106 aa, synthetic construct; identical to SEQ ID NO: 51
  REGION 1..106: note = Description of Artificial Sequence: Syntheticpolypeptide

SEQ ID NO: 507 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHLKYDPKFQVRVTMTRDTSTSTVYMELSSLRSEDTAVYYCARSGGLPDMWGQGTTVTVSS

SEQ ID NO: 508 - protein, 106 aa, synthetic construct; identical to SEQ ID NO: 51
  REGION 1..106: note = Description of Artificial Sequence: Syntheticpolypeptide

SEQ ID NO: 509 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHLKYDPKFQRRVTMTRDTSTSTVYMELSSLRSEDTAVYYCARSGGLPDMWGQGTTVTVSS

SEQ ID NO: 510 - protein, 106 aa, synthetic construct; identical to SEQ ID NO: 48
  REGION 1..106: note = Description of Artificial Sequence: Syntheticpolypeptide

SEQ ID NO: 511 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHLKYDPKFQNRVTMTRDTSTSTVYMELSSLRSEDTAVYYCARSGGLPDKWGQGTTVTVSS

SEQ ID NO: 512 - protein, 106 aa, synthetic construct; identical to SEQ ID NO: 496
  REGION 1..106: note = Description of Artificial Sequence: Syntheticpolypeptide

SEQ ID NO: 513 - protein, 116 aa, synthetic construct; identical to SEQ ID NO: 511
  REGION 1..116: note = Description of Artificial Sequence: Syntheticpolypeptide

SEQ ID NO: 514 - protein, 106 aa, synthetic construct; identical to SEQ ID NO: 48
  REGION 1..106: note = Description of Artificial Sequence: Syntheticpolypeptide

SEQ ID NO: 515 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHLKYDPKFQERVTMTRDTSTSTVYMELSSLRSEDTAVYYCARSGGLPDKWGQGTTVTVSS

SEQ ID NO: 516 - protein, 106 aa, synthetic construct; identical to SEQ ID NO: 496
  REGION 1..106: note = Description of Artificial Sequence: Syntheticpolypeptide

SEQ ID NO: 517 - protein, 116 aa, synthetic construct; identical to SEQ ID NO: 515
  REGION 1..116: note = Description of Artificial Sequence: Syntheticpolypeptide

SEQ ID NO: 518 - protein, 106 aa, synthetic construct; identical to SEQ ID NO: 51
  REGION 1..106: note = Description of Artificial Sequence: Syntheticpolypeptide

SEQ ID NO: 519 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHLKYDPKFQGRVTITRDTSASTAYMELSSLRSEDTAVYYCARSGGLPDMWGQGTTVTVSS

SEQ ID NO: 520 - protein, 106 aa, synthetic construct; identical to SEQ ID NO: 51
  REGION 1..106: note = Description of Artificial Sequence: Syntheticpolypeptide

SEQ ID NO: 521 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHLKYDPKFQGRVTITRDTSASTVYMELSSLRSEDTAVYYCARSGGLPDMWGQGTTVTVSS

SEQ ID NO: 522 - protein, 106 aa, synthetic construct; identical to SEQ ID NO: 38
  REGION 1..106: note = Description of Artificial Sequence: Syntheticpolypeptide

SEQ ID NO: 523 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHTKYDPKFQGRATITTDTSASTAYLQLSSLRSEDTAVYYCARSGGLPDVWGQGTTVTVSS

SEQ ID NO: 524 - protein, 106 aa, synthetic construct; identical to SEQ ID NO: 38
  REGION 1..106: note = Description of Artificial Sequence: Syntheticpolypeptide

SEQ ID NO: 525 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHTKYDPKFQVRATITTDTSASTAYLQLSSLRSEDTAVYYCARSGGLPDFWGQGTTVTVSS

SEQ ID NO: 526 - protein, 106 aa, synthetic construct; identical to SEQ ID NO: 38
  REGION 1..106: note = Description of Artificial Sequence: Syntheticpolypeptide

SEQ ID NO: 527 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHTKYDPKFQGRATITTDTSASTAYLQLSSLRSEDTAVYYCARSGGLPDFWGQGTTVTVSS

SEQ ID NO: 528 - protein, 106 aa, synthetic construct; identical to SEQ ID NO: 38
  REGION 1..106: note = Description of Artificial Sequence: Syntheticpolypeptide

SEQ ID NO: 529 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHTKYDPKFQGRATITTDTSASTAYLQLSSLRSEDTAVYYCARSGGLPDLWGQGTTVTVSS

SEQ ID NO: 530 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDFTLTISRLEPEDFAVYYCSQWSGNPRTFGGGTKLEIK

SEQ ID NO: 531 - protein, 116 aa, synthetic construct; identical to SEQ ID NO: 527
  REGION 1..116: note = Description of Artificial Sequence: Syntheticpolypeptide

SEQ ID NO: 532 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDFTLTISRLEPEDFAVYYCQQWSGNPRSFGGGTKLEIK

SEQ ID NO: 533 - protein, 116 aa, synthetic construct; identical to SEQ ID NO: 527
  REGION 1..116: note = Description of Artificial Sequence: Syntheticpolypeptide

SEQ ID NO: 534 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDFTLTISRLEPEDFAVYYCQQWSRNPRTFGGGTKLEIK

SEQ ID NO: 535 - protein, 116 aa, synthetic construct; identical to SEQ ID NO: 527
  REGION 1..116: note = Description of Artificial Sequence: Syntheticpolypeptide

SEQ ID NO: 536 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDFTLTISRLEPEDFAVYYCQQWKGNPRTFGGGTKLEIK

SEQ ID NO: 537 - protein, 116 aa, synthetic construct; identical to SEQ ID NO: 527
  REGION 1..116: note = Description of Artificial Sequence: Syntheticpolypeptide

SEQ ID NO: 538 - protein, 106 aa, synthetic construct; identical to SEQ ID NO: 38
  REGION 1..106: note = Description of Artificial Sequence: Syntheticpolypeptide

SEQ ID NO: 539 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHSKYDPKFQVRATITTDTSASTAYLQLSSLRSEDTAVYYCARSGGLPDFWGQGTTVTVSS

SEQ ID NO: 540 - protein, 106 aa, synthetic construct; identical to SEQ ID NO: 38
  REGION 1..106: note = Description of Artificial Sequence: Syntheticpolypeptide

SEQ ID NO: 541 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHYKYDPKFQVRATITTDTSASTAYLQLSSLRSEDTAVYYCARSGGLPDFWGQGTTVTVSS

SEQ ID NO: 542 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 543 - protein, 326 aa, synthetic construct
  REGION 1..326: note = Description of Artificial Sequence: Syntheticpolypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 544 - protein, 107 aa, synthetic construct [sequence omitted]

SEQ ID NO: 545 - protein, 25 aa, synthetic construct [sequence omitted]

SEQ ID NO: 546 - protein, 14 aa, synthetic construct
  REGION 1..14: note = Description of Artificial Sequence: Syntheticpeptide
WVRQAPGQGLEWMG

SEQ ID NO: 547 - protein, 30 aa, synthetic construct
  REGION 1..30: note = Description of Artificial Sequence: Syntheticpolypeptide
RVTMTRDTSTSTVYMELSSLRSEDTAVYYC

SEQ ID NO: 548 - protein, 11 aa, synthetic construct
  REGION 1..11: note = Description of Artificial Sequence: Syntheticpeptide
WGQGTTVTVSS

SEQ ID NO: 549 - protein, 23 aa, synthetic construct
  REGION 1..23: note = Description of Artificial Sequence: Syntheticpeptide
EIVLTQSPGTLSLSPGERATLSC

SEQ ID NO: 550 - protein, 15 aa, synthetic construct
  REGION 1..15: note = Description of Artificial Sequence: Syntheticpeptide
WYQQKPGQAPRLLIY

SEQ ID NO: 551 - protein, 32 aa, synthetic construct
  REGION 1..32: note = Description of Artificial Sequence: Syntheticpolypeptide
GIPDRFSGSGSGTDFTLTISRLEPEDFAVYYC

SEQ ID NO: 552 - protein, 10 aa, synthetic construct
  REGION 1..10: note = Description of Artificial Sequence: Syntheticpeptide
FGGGTKLEIK

SEQ ID NO: 553 - protein, 10 aa, synthetic construct
  REGION 1..10: note = Description of Artificial Sequence: Syntheticpeptide
GFDIQDTYMH

SEQ ID NO: 554 - protein, 17 aa, synthetic construct
  REGION 1..17: note = Description of Artificial Sequence: Syntheticpeptide
RIDPASGHTKYDPKFQV

SEQ ID NO: 555 - protein, 17 aa, synthetic construct
  REGION 1..17: note = Description of Artificial Sequence: Syntheticpeptide
RIEPASGHIKYDPKFQG

SEQ ID NO: 556 - protein, 17 aa, synthetic construct
  REGION 1..17: note = Description of Artificial Sequence: Syntheticpeptide
RIEPASGHIKYSPKFQG

SEQ ID NO: 557 - protein, 17 aa, synthetic construct
  REGION 1..17: note = Description of Artificial Sequence: Syntheticpeptide
RIEPASGHVKYSPKFQV

SEQ ID NO: 558 - protein, 17 aa, synthetic construct
  REGION 1..17: note = Description of Artificial Sequence: Syntheticpeptide
RIEPASGHVKYDPKFQT

SEQ ID NO: 559 - protein, 17 aa, synthetic construct
  REGION 1..17: note = Description of Artificial Sequence: Syntheticpeptide
RIDPASGHIKYDPKFQK

SEQ ID NO: 560 - protein, 17 aa, synthetic construct
  REGION 1..17: note = Description of Artificial Sequence: Syntheticpeptide
RIDPASGHVKIDPKFQV

SEQ ID NO: 561 - protein, 17 aa, synthetic construct
  REGION 1..17: note = Description of Artificial Sequence: Syntheticpeptide
RIDPASGHLKYDPKFQV

SEQ ID NO: 562 - protein, 17 aa, synthetic construct
  REGION 1..17: note = Description of Artificial Sequence: Syntheticpeptide
RIDPASGHLKYDPKFQR

SEQ ID NO: 563 - protein, 17 aa, synthetic construct
  REGION 1..17: note = Description of Artificial Sequence: Syntheticpeptide
RIDPASGHLKYDPKFQN

SEQ ID NO: 564 - protein, 17 aa, synthetic construct
  REGION 1..17: note = Description of Artificial Sequence: Syntheticpeptide
RIEPASGHLKYDPKFQE

SEQ ID NO: 565 - protein, 9 aa, synthetic construct
  REGION 1..9: note = Description of Artificial Sequence: Syntheticpeptide
ARSGGLPDV

SEQ ID NO: 566 - protein, 9 aa, synthetic construct
  REGION 1..9: note = Description of Artificial Sequence: Syntheticpeptide
ARSGGLPDW

SEQ ID NO: 567 - protein, 9 aa, synthetic construct
  REGION 1..9: note = Description of Artificial Sequence: Syntheticpeptide
ARSGGLPDM

SEQ ID NO: 568 - protein, 9 aa, synthetic construct
  REGION 1..9: note = Description of Artificial Sequence: Syntheticpeptide
ARSGGLPDK

SEQ ID NO: 569 - protein, 10 aa, synthetic construct
  REGION 1..10: note = Description of Artificial Sequence: Syntheticpeptide
RASSSVSYMY

SEQ ID NO: 570 - protein, 10 aa, synthetic construct
  REGION 1..10: note = Description of Artificial Sequence: Syntheticpeptide
GASSSVSYMY

SEQ ID NO: 571 - protein, 9 aa, synthetic construct
  REGION 1..9: note = Description of Artificial Sequence: Syntheticpeptide
QQWSGNPRT

SEQ ID NO: 572 - protein, 9 aa, synthetic construct
  REGION 1..9: note = Description of Artificial Sequence: Syntheticpeptide
QQWEGNPRT

SEQ ID NO: 573 - protein, 9 aa, synthetic construct
  REGION 1..9: note = Description of Artificial Sequence: Syntheticpeptide
QQWQGNPRT

SEQ ID NO: 574 - protein, 17 aa, synthetic construct
  REGION 1..17: note = Description of Artificial Sequence: Syntheticpeptide
RIDPASGHLKYDPKFQG

SEQ ID NO: 575 - protein, 17 aa, synthetic construct
  REGION 1..17: note = Description of Artificial Sequence: Syntheticpeptide
RIDPASGHTKYDPKFQG

SEQ ID NO: 576 - protein, 17 aa, synthetic construct
  REGION 1..17: note = Description of Artificial Sequence: Syntheticpeptide
RIDPASGHSKYDPKFQV

SEQ ID NO: 577 - protein, 17 aa, synthetic construct
  REGION 1..17: note = Description of Artificial Sequence: Syntheticpeptide
RIDPASGHYKYDPKFQV

SEQ ID NO: 578 - protein, 9 aa, synthetic construct
  REGION 1..9: note = Description of Artificial Sequence: Syntheticpeptide
ARSGGLPDV

SEQ ID NO: 579 - protein, 9 aa, synthetic construct
  REGION 1..9: note = Description of Artificial Sequence: Syntheticpeptide
ARSGGLPDM

SEQ ID NO: 580 - protein, 9 aa, synthetic construct
  REGION 1..9: note = Description of Artificial Sequence: Syntheticpeptide
ARSGGLPDF

SEQ ID NO: 581 - protein, 9 aa, synthetic construct
  REGION 1..9: note = Description of Artificial Sequence: Syntheticpeptide
ARSGGLPDL

SEQ ID NO: 582 - protein, 9 aa, synthetic construct
  REGION 1..9: note = Description of Artificial Sequence: Syntheticpeptide
SQWSGNPRT

SEQ ID NO: 583 - protein, 9 aa, synthetic construct
  REGION 1..9: note = Description of Artificial Sequence: Syntheticpeptide
QQWSGNPRS

SEQ ID NO: 584 - protein, 9 aa, synthetic construct
  REGION 1..9: note = Description of Artificial Sequence: Syntheticpeptide
QQWSRNPRT

SEQ ID NO: 585 - protein, 9 aa, synthetic construct
  REGION 1..9: note = Description of Artificial Sequence: Syntheticpeptide
QQWKGNPRT

SEQ ID NO: 586 - protein, 30 aa, synthetic construct
  REGION 1..30: note = Description of Artificial Sequence: Syntheticpolypeptide
RATITTDTSASTAYLQLSSLRSEDTAVYYC

SEQ ID NO: 587 - protein, 30 aa, synthetic construct
  REGION 1..30: note = Description of Artificial Sequence: Syntheticpolypeptide
RVTITRDTSASTVYMELSSLRSEDTAVYYC

SEQ ID NO: 588 - protein, 30 aa, synthetic construct
  REGION 1..30: note = Description of Artificial Sequence: Syntheticpolypeptide
RVTITRDTSASTAYMELSSLRSEDTAVYYC

SEQ ID NO: 589 - protein, 6 aa, synthetic construct
  REGION 1..6: note = Description of Artificial Sequence: Syntheticpeptide
HHHHHH

SEQ ID NO: 590 - protein, 9 aa, synthetic construct
  REGION 1..9: note = Description of Artificial Sequence: Syntheticpeptide
SRSGGLPDV

SEQ ID NO: 591 - protein, 10 aa, synthetic construct
  REGION 1..10: note = Description of Artificial Sequence: Syntheticpeptide
GFEIQDTYMH

SEQ ID NO: 592 - protein, 10 aa, synthetic construct
  REGION 1..10: note = Description of Artificial Sequence: Syntheticpeptide
GFDPQDTYMH

SEQ ID NO: 593 - protein, 10 aa, synthetic construct
  REGION 1..10: note = Description of Artificial Sequence: Syntheticpeptide
GFDVQDTYMH

SEQ ID NO: 594 - protein, 10 aa, synthetic construct
  REGION 1..10: note = Description of Artificial Sequence: Syntheticpeptide
GFDIGDTYMH

SEQ ID NO: 595 - protein, 10 aa, synthetic construct
  REGION 1..10: note = Description of Artificial Sequence: Syntheticpeptide
GFDISDTYMH

SEQ ID NO: 596 - protein, 10 aa, synthetic construct
  REGION 1..10: note = Description of Artificial Sequence: Syntheticpeptide
GFDIVDTYMH

SEQ ID NO: 597 - protein, 10 aa, synthetic construct
  REGION 1..10: note = Description of Artificial Sequence: Syntheticpeptide
GFDIQDAYMH

SEQ ID NO: 598 - protein, 10 aa, synthetic construct
  REGION 1..10: note = Description of Artificial Sequence: Syntheticpeptide
GFDIQDSYMH

SEQ ID NO: 599 - protein, 10 aa, synthetic construct
  REGION 1..10: note = Description of Artificial Sequence: Syntheticpeptide
GFDIQDTFMH

SEQ ID NO: 600 - protein, 10 aa, synthetic construct
  REGION 1..10: note = Description of Artificial Sequence: Syntheticpeptide
GFDIQDTYIH

SEQ ID NO: 601 - protein, 10 aa, synthetic construct
  REGION 1..10: note = Description of Artificial Sequence: Syntheticpeptide
GFDLQDTYMH

SEQ ID NO: 602 - protein, 10 aa, synthetic construct
  REGION 1..10: note = Description of Artificial Sequence: Syntheticpeptide
GFDIQDTYLH

SEQ ID NO: 603 - protein, 10 aa, synthetic construct
  REGION 1..10: note = Description of Artificial Sequence: Syntheticpeptide
RIDPASGHTK

SEQ ID NO: 604 - protein, 10 aa, synthetic construct
  REGION 1..10: note = Description of Artificial Sequence: Syntheticpeptide
RLDPASGHTK

SEQ ID NO: 605 - protein, 10 aa, synthetic construct
  REGION 1..10: note = Description of Artificial Sequence: Syntheticpeptide
RIEPASGHTK

SEQ ID NO: 606 - protein, 10 aa, synthetic construct
  REGION 1..10: note = Description of Artificial Sequence: Syntheticpeptide
RIDPESGHTK

SEQ ID NO: 607 - protein, 10 aa, synthetic construct
  REGION 1..10: note = Description of Artificial Sequence: Syntheticpeptide
RIDPAGGHTK

SEQ ID NO: 608 - protein, 10 aa, synthetic construct
  REGION 1..10: note = Description of Artificial Sequence: Syntheticpeptide
RIDPASAHTK

SEQ ID NO: 609 - protein, 10 aa, synthetic construct
  REGION 1..10: note = Description of Artificial Sequence: Syntheticpeptide
RIDPASGHIK

SEQ ID NO: 610 - protein, 10 aa, synthetic construct
  REGION 1..10: note = Description of Artificial Sequence: Syntheticpeptide
RIDPASGHLK

SEQ ID NO: 611 - protein, 10 aa, synthetic construct
  REGION 1..10: note = Description of Artificial Sequence: Syntheticpeptide
RIDPASGHVK

SEQ ID NO: 612 - protein, 7 aa, synthetic construct
  REGION 1..7: note = Description of Artificial Sequence: Syntheticpeptide
YDPKFQV

SEQ ID NO: 613 - protein, 7 aa, synthetic construct
  REGION 1..7: note = Description of Artificial Sequence: Syntheticpeptide
IDPKFQV

SEQ ID NO: 614 - protein, 7 aa, synthetic construct
  REGION 1..7: note = Description of Artificial Sequence: Syntheticpeptide
LDPKFQV

SEQ ID NO: 615 - protein, 7 aa, synthetic construct
  REGION 1..7: note = Description of Artificial Sequence: Syntheticpeptide
MDPKFQV

SEQ ID NO: 616 - protein, 7 aa, synthetic construct
  REGION 1..7: note = Description of Artificial Sequence: Syntheticpeptide
SDPKFQV

SEQ ID NO: 617 - protein, 7 aa, synthetic construct
  REGION 1..7: note = Description of Artificial Sequence: Syntheticpeptide
TDPKFQV

SEQ ID NO: 618 - protein, 7 aa, synthetic construct
  REGION 1..7: note = Description of Artificial Sequence: Syntheticpeptide
VDPKFQV

SEQ ID NO: 619 - protein, 7 aa, synthetic construct
  REGION 1..7: note = Description of Artificial Sequence: Syntheticpeptide
YIPKFQV

SEQ ID NO: 620 - protein, 7 aa, synthetic construct
  REGION 1..7: note = Description of Artificial Sequence: Syntheticpeptide
YNPKFQV

SEQ ID NO: 621 - protein, 7 aa, synthetic construct
  REGION 1..7: note = Description of Artificial Sequence: Syntheticpeptide
YRPKFQV

SEQ ID NO: 622 - protein, 7 aa, synthetic construct
  REGION 1..7: note = Description of Artificial Sequence: Syntheticpeptide
YSPKFQV

SEQ ID NO: 623 - protein, 7 aa, synthetic construct
  REGION 1..7: note = Description of Artificial Sequence: Syntheticpeptide
YDPKFRV

SEQ ID NO: 624 - protein, 7 aa, synthetic construct
  REGION 1..7: note = Description of Artificial Sequence: Syntheticpeptide
YDPKFQA

SEQ ID NO: 625 - protein, 7 aa, synthetic construct
  REGION 1..7: note = Description of Artificial Sequence: Syntheticpeptide
YDPKFQD

SEQ ID NO: 626 - protein, 7 aa, synthetic construct
  REGION 1..7: note = Description of Artificial Sequence: Syntheticpeptide
YDPKFQE

SEQ ID NO: 627 - protein, 7 aa, synthetic construct
  REGION 1..7: note = Description of Artificial Sequence: Syntheticpeptide
YDPKFQG

SEQ ID NO: 628 - protein, 7 aa, synthetic construct
  REGION 1..7: note = Description of Artificial Sequence: Syntheticpeptide
YDPKFQH

SEQ ID NO: 629 - protein, 7 aa, synthetic construct
  REGION 1..7: note = Description of Artificial Sequence: Syntheticpeptide
YDPKFQK

SEQ ID NO: 630 - protein, 7 aa, synthetic construct
  REGION 1..7: note = Description of Artificial Sequence: Syntheticpeptide
YDPKFQL

SEQ ID NO: 631 - protein, 7 aa, synthetic construct
  REGION 1..7: note = Description of Artificial Sequence: Syntheticpeptide
YDPKFQM

SEQ ID NO: 632 - protein, 7 aa, synthetic construct
  REGION 1..7: note = Description of Artificial Sequence: Syntheticpeptide
YDPKFQN

SEQ ID NO: 633 - protein, 7 aa, synthetic construct
  REGION 1..7: note = Description of Artificial Sequence: Syntheticpeptide
YDPKFQP

SEQ ID NO: 634 - protein, 7 aa, synthetic construct
  REGION 1..7: note = Description of Artificial Sequence: Syntheticpeptide
YDPKFQR

SEQ ID NO: 635 - protein, 7 aa, synthetic construct
  REGION 1..7: note = Description of Artificial Sequence: Syntheticpeptide
YDPKFQS

SEQ ID NO: 636 - protein, 7 aa, synthetic construct
  REGION 1..7: note = Description of Artificial Sequence: Syntheticpeptide
YDPKFQT

SEQ ID NO: 637 - protein, 7 aa, synthetic construct
  REGION 1..7: note = Description of Artificial Sequence: Syntheticpeptide
LGGLPDV

SEQ ID NO: 638 - protein, 7 aa, synthetic construct
  REGION 1..7: note = Description of Artificial Sequence: Syntheticpeptide
SAGLPDV

SEQ ID NO: 639 - protein, 7 aa, synthetic construct
  REGION 1..7: note = Description of Artificial Sequence: Syntheticpeptide
SGGAPDV

SEQ ID NO: 640 - protein, 7 aa, synthetic construct
  REGION 1..7: note = Description of Artificial Sequence: Syntheticpeptide
SGGMPDV

SEQ ID NO: 641 - protein, 7 aa, synthetic construct
  REGION 1..7: note = Description of Artificial Sequence: Syntheticpeptide
SGGLPEV

SEQ ID NO: 642 - protein, 7 aa, synthetic construct
  REGION 1..7: note = Description of Artificial Sequence: Syntheticpeptide
SGGLPDK

SEQ ID NO: 643 - protein, 7 aa, synthetic construct
  REGION 1..7: note = Description of Artificial Sequence: Syntheticpeptide
SGGLPDM

SEQ ID NO: 644 - protein, 7 aa, synthetic construct
  REGION 1..7: note = Description of Artificial Sequence: Syntheticpeptide
SGGLPDQ

SEQ ID NO: 645 - protein, 7 aa, synthetic construct
  REGION 1..7: note = Description of Artificial Sequence: Syntheticpeptide
SGGLPDR

SEQ ID NO: 646 - protein, 7 aa, synthetic construct
  REGION 1..7: note = Description of Artificial Sequence: Syntheticpeptide
SGGLPDS

SEQ ID NO: 647 - protein, 7 aa, synthetic construct
  REGION 1..7: note = Description of Artificial Sequence: Syntheticpeptide
SGGLPDT

SEQ ID NO: 648 - protein, 7 aa, synthetic construct
  REGION 1..7: note = Description of Artificial Sequence: Syntheticpeptide
SGGLPDW

SEQ ID NO: 649 - protein, 10 aa, synthetic construct
  REGION 1..10: note = Description of Artificial Sequence: Syntheticpeptide
WASSSVSYMY

SEQ ID NO: 650 - protein, 10 aa, synthetic construct
  REGION 1..10: note = Description of Artificial Sequence: Syntheticpeptide
RASSSVIYMY

SEQ ID NO: 651 - protein, 10 aa, synthetic construct
  REGION 1..10: note = Description of Artificial Sequence: Syntheticpeptide
RASSSVSFMY

SEQ ID NO: 652 - protein, 10 aa, synthetic construct
  REGION 1..10: note = Description of Artificial Sequence: Syntheticpeptide
RASSSVSYLY

SEQ ID NO: 653 - protein, 10 aa, synthetic construct
  REGION 1..10: note = Description of Artificial Sequence: Syntheticpeptide
RASSSVSYMR

SEQ ID NO: 654 - protein, 7 aa, synthetic construct
  REGION 1..7: note = Description of Artificial Sequence: Syntheticpeptide
AKSNLAS

SEQ ID NO: 655 - protein, 7 aa, synthetic construct
  REGION 1..7: note = Description of Artificial Sequence: Syntheticpeptide
ATPNLAS

SEQ ID NO: 656 - protein, 7 aa, synthetic construct
  REGION 1..7: note = Description of Artificial Sequence: Syntheticpeptide
ATENLAS

SEQ ID NO: 657 - protein, 7 aa, synthetic construct
  REGION 1..7: note = Description of Artificial Sequence: Syntheticpeptide
ATSLLAS

SEQ ID NO: 658 - protein, 7 aa, synthetic construct
  REGION 1..7: note = Description of Artificial Sequence: Syntheticpeptide
ATSPLAS

SEQ ID NO: 659 - protein, 7 aa, synthetic construct
  REGION 1..7: note = Description of Artificial Sequence: Syntheticpeptide
ATSNLTS

SEQ ID NO: 660 - protein, 9 aa, synthetic construct
  REGION 1..9: note = Description of Artificial Sequence: Syntheticpeptide
HQWSGNPRT

SEQ ID NO: 661 - protein, 9 aa, synthetic construct
  REGION 1..9: note = Description of Artificial Sequence: Syntheticpeptide
NQWSGNPRT

SEQ ID NO: 662 - protein, 9 aa, synthetic construct
  REGION 1..9: note = Description of Artificial Sequence: Syntheticpeptide
QQSSGNPRT

SEQ ID NO: 663 - protein, 9 aa, synthetic construct
  REGION 1..9: note = Description of Artificial Sequence: Syntheticpeptide
QQWDGNPRT

SEQ ID NO: 664 - protein, 9 aa, synthetic construct
  REGION 1..9: note = Description of Artificial Sequence: Syntheticpeptide
QQWHGNPRT

SEQ ID NO: 665 - protein, 9 aa, synthetic construct
  REGION 1..9: note = Description of Artificial Sequence: Syntheticpeptide
QQWNGNPRT

SEQ ID NO: 666 - protein, 9 aa, synthetic construct
  REGION 1..9: note = Description of Artificial Sequence: Syntheticpeptide
QQWVGNPRT

SEQ ID NO: 667 - protein, 9 aa, synthetic construct
  REGION 1..9: note = Description of Artificial Sequence: Syntheticpeptide
QQWSANPRT

SEQ ID NO: 668 - protein, 9 aa, synthetic construct
  REGION 1..9: note = Description of Artificial Sequence: Syntheticpeptide
QQWSDNPRT

SEQ ID NO: 669 - protein, 9 aa, synthetic construct
  REGION 1..9: note = Description of Artificial Sequence: Syntheticpeptide
QQWSQNPRT

SEQ ID NO: 670 - protein, 9 aa, synthetic construct
  REGION 1..9: note = Description of Artificial Sequence: Syntheticpeptide
QQWSSNPRT

SEQ ID NO: 671 - protein, 9 aa, synthetic construct
  REGION 1..9: note = Description of Artificial Sequence: Syntheticpeptide
QQFSGNPRT

SEQ ID NO: 672 - protein, 9 aa, synthetic construct
  REGION 1..9: note = Description of Artificial Sequence: Syntheticpeptide
QQHSGNPRT

SEQ ID NO: 673 - protein, 9 aa, synthetic construct
  REGION 1..9: note = Description of Artificial Sequence: Syntheticpeptide
QQISGNPRT

SEQ ID NO: 674 - protein, 9 aa, synthetic construct
  REGION 1..9: note = Description of Artificial Sequence: Syntheticpeptide
QQPSGNPRT

SEQ ID NO: 675 - protein, 9 aa, synthetic construct
  REGION 1..9: note = Description of Artificial Sequence: Syntheticpeptide
QQRSGNPRT

SEQ ID NO: 676 - protein, 9 aa, synthetic construct
  REGION 1..9: note = Description of Artificial Sequence: Syntheticpeptide
QQYSGNPRT

SEQ ID NO: 677 - protein, 9 aa, synthetic construct
  REGION 1..9: note = Description of Artificial Sequence: Syntheticpeptide
QQWSGHPRT

SEQ ID NO: 678 - protein, 9 aa, synthetic construct
  REGION 1..9: note = Description of Artificial Sequence: Syntheticpeptide
QQWSGLPRT

SEQ ID NO: 679 - protein, 9 aa, synthetic construct
  REGION 1..9: note = Description of Artificial Sequence: Syntheticpeptide
QQWSGQPRT

SEQ ID NO: 680 - protein, 9 aa, synthetic construct
  REGION 1..9: note = Description of Artificial Sequence: Syntheticpeptide
QQWSGMPRT

SEQ ID NO: 681 - protein, 7 aa, synthetic construct
  REGION 1..7: note = Description of Artificial Sequence: Syntheticpeptide
SGGLPDH

SEQ ID NO: 682 - protein, 7 aa, synthetic construct
  REGION 1..7: note = Description of Artificial Sequence: Syntheticpeptide
SGGLPDF

SEQ ID NO: 683 - protein, 7 aa, synthetic construct
  REGION 1..7: note = Description of Artificial Sequence: Syntheticpeptide
SGGSPDV

SEQ ID NO: 684 - protein, 9 aa, synthetic construct
  REGION 1..9: note = Description of Artificial Sequence: Syntheticpeptide
QQWAGNPRT

SEQ ID NO: 685 - protein, 9 aa, synthetic construct
  REGION 1..9: note = Description of Artificial Sequence: Syntheticpeptide
QQWYGNPRT

SEQ ID NO: 686 - protein, 9 aa, synthetic construct
  REGION 1..9: note = Description of Artificial Sequence: Syntheticpeptide
QQWFGNPRT

SEQ ID NO: 687 - protein, 124 aa, synthetic construct
  REGION 1..124: note = Description of Artificial Sequence: Syntheticpolypeptide
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 688 - protein, 121 aa, synthetic construct
  REGION 1..121: note = Description of Artificial Sequence: Syntheticpolypeptide
EVQLLESGGGLVQPGKSLRLSCAVSGFTFSTYGMNWVRQAPGKGLEWVSSISGTGRTTYHADSVQGRFTVSRDNSKNILYLQMNSLRADDTAVYFCTKERGDYYYGVFDYWGQGTLVTVSS

SEQ ID NO: 689 - protein, 124 aa, synthetic construct
  REGION 1..124: note = Description of Artificial Sequence: Syntheticpolypeptide
QVTLKESGPALVKPTQTLTLTCTFSGFSLSTSNMGVVWIRQPPGKALEWLAHILWDDREYSNPALKSRLTISKDTSKNQVVLTMTNMDPVDTATYYCARMSRNYYGSSYVMDYWGQGTLVTVSS

SEQ ID NO: 690 - protein, 122 aa, synthetic construct
  REGION 1..122: note = Description of Artificial Sequence: Syntheticpolypeptide
QIQLVQSGPELKKPGETVKISCKASGYTFTTYGMSWVKQAPGKGLKWMGWMNTYSGVTTYADDFKGRFAFSLETSASTAYMQIDNLKNEDTATYFCAREGYVFDDYYATDYWGQGTSVTVSS

SEQ ID NO: 691 - protein, 107 aa, synthetic construct
  REGION 1..107: note = Description of Artificial Sequence: Syntheticpolypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 692 - protein, 106 aa, synthetic construct
  REGION 1..106: note = Description of Artificial Sequence: Syntheticpolypeptide
DIQMTQSPSTLSASVGDRVTITCRASQTISSWLAWYQQTPEKAPKLLIYAASNLQSGVPSRFSGSGSGTEFTLTISSLQPDDFATYYCQQYHRSWTFGQGTKVEIT

SEQ ID NO: 693 - protein, 106 aa, synthetic construct
  REGION 1..106: note = Description of Artificial Sequence: Syntheticpolypeptide
DIQLTQSPSFLSASVGDRVTITCSASSSVNYMHWYQQKPGKAPKLLIYSTSNLASGVPSRFSGSGSGTEFTLTISSLQPEDFATYYCHQWNNYGTFGQGTKVEIKR

SEQ ID NO: 694 - protein, 112 aa, synthetic construct
  REGION 1..112: note = Description of Artificial Sequence: Syntheticpolypeptide
DVLMTQTPLSLPVSLGDQASISCRSSQNIVHSDGNTYLEWYLQKPGQSPKLLIYKVSNRFSGVPDRFSGSGSGTDFTLKISRVEAEDLGIYYCFQGSHVPLTFGAGTKLELK